(19) United States
(12) Patent Application Publication
(10) Pub. No.: US 2008/0311567 A1
(43) Pub. Date: Dec. 18, 2008

(54) Tumor Markers for Use in the Diagnosis of Colorectal Carcinomas and/or Metastases Originating Therefrom

(75) Inventors: Wolfgang Michael Bruckl, Buckenhof (DE); Axel Wein, Erlangen (DE); Marc Munnes, Erkrath (DE); Ralph Markus Wirtz, Cologne (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: FRIEDRICH-ALEXANDER- UNIVERSITAT ERLANGEN- NURNBER, Erlangen (DE)

(21) Appl. No.: 11/659,250

(22) PCT Filed: Jul. 29, 2005
(86) PCT No.: PCT/EP05/08274

§ 371 Date: Apr. 15, 2008

(30) Foreign Application Priority Data
Aug. 4, 2004 (DE) 10 2004 037 860.6

(51) Int. Cl. C12Q 1/68

(52) U.S. Cl. 435/6

(57) ABSTRACT

The invention relates to a method (i) for detecting a carcinoma, especially an adenocarcinoma, preferably a gastrointestinal carcinoma and more preferably a colorectal carcinoma, (ii) for predicting metastases, preferably liver metastases, depending on a primary colon carcinoma and/or (iii) for predicting the response of metastases to a 5-fluorouracil-containing chemotherapy. The inventive method comprises determining a gene expression profile of 120 marker genes or a selection thereof.

The invention relates to a method (i) for detecting a carcinoma, especially an adenocarcinoma, preferably a gastrointestinal carcinoma and more preferably a colorectal carcinoma, (ii) for predicting metastases, preferably liver metastases dependent on a primary colon carcinoma and/or (iii) for predicting the response of metastases to a 5-fluorouracil-containing chemotherapy, which method comprises determining a gene expression profile of 120 marker genes or a selection thereof. The 120 genes are defined by their sequences, as represented in SEQ ID NOs: 1-181. Also disclosed are kits for carrying out the method of the invention and diagnostic kits. Additional embodiments of the invention relate to the use of the marker genes disclosed herein and/or the combinations of marker genes disclosed herein. Furthermore, the invention relates to the use of non-human mammals or cells cultured therefrom, whose hereditary material was attached in reference to one or more of the genes indicated in this method. The non-human mammals or the cultured cells whose hereditary material was attached can be used particularly for tests of alternative therapies or therapeutics in reference to primary colorectal carcinomas and metastases derived therefrom.

Colorectal carcinomas represent the third most frequent tumor entity in western countries. In Germany, approximately 50,000 patients fall ill with colorectal carcinomas per year. Annually, in approximately 20,000 patients with colorectal carcinoma, lung or liver metastases occurred synchronously or metachronously. From the technical point of view, in Germany, a curative metastasis resection (RO) which is associated with a 5-year survival rate of approximately 30% is possible in approximately 4,000 of the 20,000 patients with distant metastases.

In the remaining 16,000 patients, a resection is not possible for a great variety of reasons (multimodular, unfavorable localization on vessels and bile ducts, ektrahepatic). In these cases, palliative therapy options are indicated. The purpose of the palliative treatment, in addition to maintaining a good quality of life, is to increase the survival time. Currently many therapeutic regimens based on 5-FU/folic acid are available. A response rate of approximately 40% can be achieved, without adding additional substances. In several studies, the combination of 5-FU/folic acid with irinotecan or oxaliplatin led to an improvement of the response rate up to 50% of the patients treated. In addition, additional substances are now being tested in the palliative therapy of colorectal carcinoma, such as, for example, antibodies against VEGR and EGF-R or so-called “small molecules” against the tyrosine kinase domain of VEGF-R or EGF-R. At this time, the final usefulness of these new substances will not be determined until additional investigations are carried out in the context of a preclinical trial and clinical phase I-III studies. Owing to the multitude of relevant study results produced with different regimens, no generally accepted standard regimen can be indicated for the first treatment.

The response to the therapy is evaluated according to the WHO guidelines (World Health Organization. Handbook for Reporting Results of Cancer Treatment. WHO Offset Publication No. 48. Geneva: WHO 1979). Patients who present a complete remission (CR, complete remission) or a partial remission (PR, partial remission) in imaging procedures are called “responders,” while patients who present a stable tumor course (SD, stable disease) or a progressive tumor course (PD, progressive disease) in imaging are called “non-responders.” In colorectal carcinoma, response to the chemotherapy correlates with the survival of the patients. The factors which can predict the response to chemotherapy are called predictive markers. an individualized therapy could be made available based on predictive markers. In spite of numerous investigations of various types in this field, the molecular predictive markers described in the literature to date are not sufficient for metastasized colorectal cancer.

Watson (2003) Clin. Sci. 104:537-545 shows in a genotyping study that polymorphisms of the apolipoprotein E gene influence the risk of falling ill with colorectal carcinoma. According to the investigation, persons with a ε2/ε2 genotype, in contrast to the widespread ε3/ε3 genotype, present an elevated risk of falling ill with colorectal carcinoma, with a clear gender specificity in reference to the disease. Sladek (2002) Cancer chemother. Pharmacol. 49:309-321 and Sreerama (2001) Cancer Chemother. Pharmacol. 47:255-362 point out the possibility of using the determination of the cellular level of expression of the aldehyde dehydrogenases ALDH1A1 and ALDH3A1 to predict the response of breast cancer to a cyclophosphamide- (or other oxazaphosphorines, for example: ifosfamide)-based chemotherapy. Kemmner (1994) Clin. Exp. Metastasis 12:245-254 states that in human colorectal tumors the expression of two sialyl transferases is elevated compared to normal tissue, and that a quantitative determination of the specific sialyl transferase activity could therefore be suitable for detecting and monitoring colon carcinoma. Sotiropoulou (2002 Mol. Med. 8:42-55 identifies a new sialyl transferase gene (SIATL1) whose expression is downregulated in human breast tumor tissue cell lines in comparison to normal breast tissue cell lines. The abnormal expression and enzymatic activity of sialyl transferases in tumor cells leads to the formation of tumor-associated carbohydrate antigens which can be used, particularly in immunotherapy. Dolnick (1996) Advan. Enzym Regul. 36:165-180 and Dolnick (1996) Cancer Res. 56:3207-3210 detect two gene products of the rThymidylate synthase genes, called rTSoα and rTSβ. In the two cell lines H630-1 and H630-10, modified thymidylate synthase activity is observed. In cell lines, Maxwell (2003) Cancer Res. 63:4602-4606 detects a change in the gene expression in 619 genes using the DNA-microchip technology. The investigations were carried out on MCF-7 breast cancer cells, as well as on H630- and H630-R10 colon carcinoma cell lines. An expression-activating effect of 5-FU and an elevated expression of the spermin/spermidine acetyltransferase genes, of the annexin II genes, of the thymosin β-10 genes, of the chaperonin-10 gene and, particularly, of the MAT-8 genes are described. Biran (1986) Clin. Pathol. 39:794-797, in a study of 160 lung cancer patients, measures the concentration of the serum amyloid A and indicates that an active disease is connected with a high titer in comparison to the inactive stage of the disease. Serial investigations produced a good agreement between the change in the serum amyloid A concentration and the clinical course. Bruno (2003) Clin. Cancer Res. 9:1077-1082 investigated 180 lung cancer patients (NSLCL [sic; NSCLC]) and claims that α₁-acidic glycoprotein is a protein for predicting treatment effects in lung cancer patients. O'Hanlon (2002) Anticancer Res. 22:1289-1293, in a study of acute phase proteins, detects concentrations of C-reactive protein and of serum amyloid A in 92 patients with breast cancer and 31 patients with benign breast tissue change. Serial investigations yielded significantly higher concentrations of the two proteins in patients in UICC [International Union Against Cancer] stage IV, and particularly in T4 ulcerating tumors. Simpson (1995) Clin. Exp. Immunol. 99:143-147 treated 24 patients who suffered from metastasizing colorectal carcinoma with a combination therapy consisting of recombinant interleukin 2 (rIL-2), followed by 5-fluorouracil and folic acid. The therapy course was investigated by measuring the concentrations of C-reactive protein, retinol binding protein, alpha 1-antitrypsin, transferrin and albumin. Only 7 of the 24 patients responded to the rIL-2 based therapy. In 67 patients with colorectal carcinoma, Glojnaric (2001) Clin. Chem. Lab. Med. 39:129-133 determined the concentrations of serum amyloid A protein, C-reactive protein, α₁-antichymotrypsin and α₁-acidic glycoprotein at 12 times (before the operation, after the operation, and before 9 chemotherapy cycles that are not specified in greater detail). Of the four acute phase proteins investigated, the serum amyloid A protein was found to present the highest specificity and sensitivity. Therefore, the author proposes only the serum amyloid A protein as a reliable, nonspecific tumor marker for colorectal carcinoma in daily clinical practice. McMillan (2003) Br. J. Surg. 90:215-219 investigated a change in the concentration of the C-reactive protein in 174 patients with colorectal carcinoma before and after the resection. The author observed that the validity of this parameter is low. Koeffler (2003) Clin. Cancer Res. 9:1-9 describes the different views that exist regarding the connection between the peroxisome proliferative activated receptor-γ (PPAR_{γ}) and cancer. The gene is activated only by the binding of a ligand. Some research results suggest that PPAR_{γ} functions as tumor suppressor, whereas other research results on murine models support the notion that PPAR_{γ} ligands promote the formation of cancer under certain conditions. Gupta (2002) Am. J. Physiol. Gastrointest. Liver Physiol. 283:G266-G269 investigated the question whether an activation of the peroxosome proliferative activated receptor-γ (PPAR_{γ}) accelerates or decreases the growth of colorectal carcinomas, because an activation of the peroxosome proliferative activated receptor-γ (PPAR_{γ}) has a negative influence, due to ligand binding, on the proliferation of cultured colorectal cancer cells or of colorectal carcinomas in the murine model. In 81 patients with metastasizing malignant melanoma and in 50 normal volunteers, Mouawad (2002) Melanoma Res. 12:343-348 determined the expression level of caspase-1 in order to establish a correlation between this enzyme which participates in apoptotic processes and the pathological condition of the patient.

The patients received cisplatin, recombinant interleukin-2, and α-interferon. The average caspase-1 level in melanoma patients was clearly above that of the control group. De Vita (1993) Eur. J. Cancer 29:887-893 describes some Hox genes in primary colon tumors and derived hepatic metastases which present an elevated expression level, which suggested a connection between this gene and the growth of the colon tumors. Kawazoe (2002) Develop. Growth Differ. 44:77-84, in in-situ hybridization studies, demonstrated the expression of different Hox genes on postembryonic day 12.5 in the intestine of the mouse. For the genes of the HoxD gene, they demonstrated the expression of HoxD-4, HoxD-8 and HoxD-9 in reference to expression in the colon. Nunes (2003) Pesqui. Odontol. Bras. 17:94-98, combined the results of the current literature to formulate the thesis that there are many connections between normal development and tumor formation. Cillo (1994-95) Invasion Metastasis 14, 38-39 investigated the expression pattern of the Hox gene in healthy human organs, in kidney and colon tumors, small cell bronchial carcinomas (SCLC [small cell lung cancer]), and metastasizing variants. He arrives at the result that Hox genes function as a network for the regulation of transcription and participate in the processes of cell-to-cell communication during normal morphogenesis. Wang (2002) Cancer Letters 179:71-77 compared the expression of the CASK and Reelin genes in human gastric, colon and esophageal carcinomas by RT-PCR, immunohistochemical methods, and western blot analysis. In 87.5% of the esophageal carcinomas investigated, an elevated expression of the CASK and Reelin genes was found in comparison to the healthy esophageal tissue. In 50% of the gastric carcinomas and 64.3% of the colon carcinomas, an increase in the CASK expression level compared to the normal tissue was demonstrated, whereas the Reelin level underwent no change. The author arrives at the result that the CASK gene is involved in the tumorogenesis of the esophagus by influencing the transcription of the Reelin genes. Nevertheless, the exact function of the CASK gene and the Reelin gene and their mutual interaction still remain unclear. Kuno (1997) JBC 272:556-562 reports on ADAMTS-1 from the ADAM gene family. Investigations showed that the intravenous administration of lipopolysaccharides to mice activates the transcription of the ADAMTS-1 genes selectively in the kidney and the heart. From these results, the authors concluded that the ADAMTS-1 gene participates in different inflammatory processes, as well as in the development of tumor-related cachexia. Iruela-Arispe (2003) Ann. NY Acad. Sci. 995:183-190 isolated a secreted metalloproteinase (METH1/ADAMTS1) and showed that in vitro it inhibits the growth of endothelial cells and in vivo blocks the neovascular response induced by growth factors. The authors found indications that two domains of the protein are needed to develop the antiangiagenesis/antitumor activity of ADAMTS1. Rathnakumar (2000) Indian J. Cancer. 37:23-26 points out that numerous biochemical tests are carried out for diagnosing liver metastases before and after surgery. However, the value of these biochemical methods has decreased following the recent introduction of high-resolution imaging procedures such as computer tomography or ultrasonography. Different biochemical markers have been proposed for identifying liver metastases, and in this study the author argues in favor determining the 5′-nucleotidase content because he considers this gene the most sensitive marker for liver metastases. Eroglu (2000) Med. Oncol. 17:319-324 contends that close attention should be paid to enzymatic studies for the characterization of colorectal tumors, and that little is known on the connection between the level of the key enzymes of the purinenucleotide signal pathway and some clinical and biological indicators of invasiveness and aggression of tumors. In his study, the author determined the content of adenosine deaminase (ADA) and 5′-nucleotidase (5′-NT) in healthy tissue and in tumor tissue of 38 patients with colorectal carcinoma. The result of the study was that the enzyme content in the primary tumor is clearly higher than that in the normal mucosa. Galmarini (2002) Brit. J. Haematol. 117:860-868 investigated the resistance mechanism against cytarabine in acute myeloid leukemia (AML). He observed that the expression of 5′-nucleotidase, HENT1 and DNA polymerase a at the time of the diagnosis causes cytarabine resistance in patients with acute myeloid leukemia. Wiksten (2003) Oncology 64:245-250 investigated the expression of tenascin-C in the connective tissue of 314 gastric carcinoma patients in an immunohistochemical study. Tenascin-C is an extracellular hexameric matrix glycoprotein which is expressed during embryonic development and in proliferative processes such as wound healing or tumorogenesis. The author discovered that it appears that the tenascin-C expression correlates with the survival of patients with stomach cancer, but does not provide significant prognostic information. Riedl (1995) Int. J. Cancer 64:65-69, in a study of 118 patients with colorectal carcinoma and a control group of 51 healthy persons, determined the serum content of tenascin. It was found that patients with colorectal carcinoma present a significantly higher serum level of tenascin than the control group. In addition, it was shown that, in patients with distant metastases, the serum level of tenascin is clearly higher than that of patients without distant metastases. Emoto (2001) Cancer 92:1419-1426, in a study, investigated the correlation of the overexpression of annexin II and tenascin-C in colorectal carcinomas. Using immunohistochemical methods, he measured the expression of both genes in 105 primary colorectal tumors. The results show that both annexin II and tenascin-C are overexpressed in advanced colorectal carcinomas. Gu (2004) Int. J. Oncol 24:671-678, in a study, investigated the question whether the expression of IMP-1, -2 and -3 in ovarian epithelial tumors is connected with the survival rate of the patients. For this purpose, the expression level of the IMP gene was determined with a semiquantitative PCR procedure in 59 ovarian epithelial carcinomas and 7 normal ovaries. Expression of the IMP gene was observed in all the tumor tissues investigated, including cancer of the breast, lung, colon, prostate and ovary, but not cancer of the pancreas. Riede (1998) Langenbecks Arch. Chir. Suppl. Kongressbd. 115:299-302 analyzed the expression and the regulation of the expression of the MUC2 gene in normal colon tissue, colon carcinoma tissue and metastasis tissue. The author noted that the MUC2 expression was significantly lower in metastases than in the normal mucosa and in the primary tumor, and was apparently not connected with the normal metastasis process. Manne (2000) Clin. Canc. Res. 6:4017-4025 investigated the prognostic value of the correlation between the expression of the MUC1 and MUC2 gene in colorectal adenocarcinomas and the aggressiveness of the colorectal adenocarcinoma in reference to the differences within the population. The expression of MUC1 and MUC2 was determined by immunohistochemical methods in 166 samples of colorectal adenocarcinoma, of which 58 were of Afro-American and 108 of Caucasian origin. No prognostic use of MUC2 for Caucasians or Afro-Americans was detected in this study. Akyurek (2002) Pathol. Res. Pract. 198:665-675 considered a possible connection between the MUC1 and MUC2 expression and the survival time of 143 gastric carcinoma patients. The author summarized his study with the result that the MUC1 expression represents a usable prognostic factor for predicting the survival probability of gastric carcinoma patients, whereas the role of the MUC2 expression remains unclear. Baba (2000) Cancer Res. 60:6886-6889 claims that the activation of the Ki-Ras signal pathway represents a factor which leads to the overexpression of epiregulin in human colon cancer cells. In addition, he is of the opinion that epiregulin plays a decisive role in vivo in tumorogenesis in humans. Dionigi (2000) Am. J. Clin. Path. 114:111-122, using immunostains, investigated 23 ovarian metastases of colorectal primary tumors and 23 primary ovarian carcinomas in order to establish criteria to allow the differential diagnosis of the two cancer types. In the immunostaining methods, antibodies against anti-gastric M1 antigen, CA125, vimentin, estrogen and progesterone receptors, cytokeratins 7 and 20, alpha-inhibin, and cathepsin E were used. The result of the study was that the immune reaction of cytokeratin 20 and the absence of an immune reaction against the anti-gastric M1 antigen, CA125 and cytokeratin 7 allow the differential diagnosis of ovarian metastases of colorectal primary tumors on the one hand and primary ovarian carcinomas on the other hand. DePrimo (2003) BMC Cancer 3:3 uses, in his study, a method based on a microarray to establish a tumor gene profile from a blood sample. In his investigations, he uses monocytes from the blood of 23 patients with advanced colorectal carcinoma who were in phase III clinical treatment with SU5416. During the course of the study, a screening was carried out to determine changes in the expression profile which were the result of the treatment with SU5416. The author identified 4 transcripts which are suitable for the classification of the patients according to the treatment method (CD24, lactoferrin, lipocallin [sic; lipocalin] 2 and MMP-9), but was unable to rule out with certainty that the altered expression of these 4 genes was also caused by the administration type or concomitant drugs. Mariadson (2003) Cancer Res. 63:8791-8812, in a study based on a microarray of 30 colon tumor cell lines, established an expression profile of 430 genes which provides valid information regarding the response of the tumor to different chemotherapeutic. In reference to the response of colon cancer to a 5-FU therapy, he focused his selection on 50 genes.

The prior art thus already makes available markers for various proliferative diseases. However, it is problematic to make available a reliable diagnosis which can lead to individual therapies, and particularly to higher response rates to therapeutic agents.

The technical problem is solved by provision of the embodiments disclosed herein, and particularly by the claims that characterize the invention.

The invention therefore comprises a method for

(a) detecting a colorectal carcinoma;

(b) prediction of metastases dependent upon a primary colorectal carcinoma; and/or

(c) prediction of the response, particularly of metastases, to a 5-FU containing therapy; comprising the determination of a gene expression profile of 120 marker genes (SEQ ID NO: 1 to SEQ ID NO: 181) or a selection therefrom.

The invention relates to the expression profiles of certain genes which are of significance in carcinomas, particularly adenocarcinomas, preferably in gastrointestinal carcinomas, and more preferably in primary colorectal carcinomas and metastases derived therefrom, preferably liver metastases. The content of the invention also relates to the advantage derived from the knowledge of this expression, and the preparation of expression profiles for the possible diagnosis, prognosis, prediction of the response to the therapeutic measures, particularly, for example, chemotherapy, and preferably a 5-FU-containing chemotherapy and/or combination therapy. Surprisingly, in this embodiment of the invention in particular, it is possible to use markers which are defined in the invention and which can be determined by the simplest of methods from/in body fluids such as blood or blood serum, but also, for example, (peritoneal) ascites or lymphatic fluid. Furthermore, the present invention can be used to observe the course of a therapy of carcinomas, particularly of primary colorectal carcinomas and metastases derived therefrom, preferably liver metastases.

The 120 genes (marker genes) of the invention are defined particularly in Table D, and they are represented by their corresponding coding sequence(s). Thus, the corresponding, individual (marker) genes are characterized partially by several SEQ ID NOs. The individual sequences of the 120 defined marker genes represent, for example, variants, particularly splice variants or genes with higher homology.

In connection with this invention, the expression “colorectal carcinoma” denotes particularly polypoid, plateau-shaped, ulcerating and flat (scirrhoid) forms which are classified histologically into solid, mucus-producing or glandular adenocarcinomas, signet ring cell carcinomas, squamous, adenosquamous, cribriform pavement epithelium-like or undifferentiated carcinomas according to the WHO classification (Becker, Hohenberger, Junginger, Schlag. Surgical Oncology. Thieme, Stuttgart 2002). As mentioned above, the methods according to the invention are not limited to colorectal carcinomas.

In connection with this invention, the term “adenocarcinoma” relates to a cancerous tumor which starts from an epithelial cell layer of the glandular portion of a mucous membrane. The adenocarcinoma represents a separate, morphologically clearly delimited part of the carcinomas.

The term “detection” of a carcinoma, particularly a colorectal carcinoma, comprises particularly the in vitro determination of a potential (colorectal) carcinoma. This detection is preferably an early detection. The term “early detection,” in connection with the invention, indicates that a (colorectal) carcinoma can be detected as early as possible in its genesis, development and/or symptomatic manifestation. “Early detection” therefore comprises the detection of (colorectal) carcinomas in an early stage of the disease, when the disease is still limited to the tumor, and no metastasizing into the lymph vessels/lymph nodes (L0, N0), other organs (M0), or vascular invasions (V0) have occurred, although it is not limited to that stage. “Early detection” relates, among other factors, to the diagnostic analysis in preventive care investigations, and also in routine investigations or follow-up investigations, for example, after the performance of the resection of an already present primary tumor.

The term “prediction,” in the context of this invention, denotes the determination of the biological, biomedical condition of tissue or individual cells, particularly the determination whether a given tissue/given cell has undergone a proliferative change. As part of this invention, a determination is made to establish whether a potential metastasis, preferably a liver metastasis, presents in fact a proliferative change and/or is in fact a secondary disease focus resulting from a primary proliferative and/or tumorigenic disease. In the context of the invention, the primary proliferative disease is preferably a colorectal carcinoma, and the secondary disease focus is a metastasis, preferably a liver metastasis.

The term “prediction,” in connection with the method according to the invention for determining the response of a tumor and particularly of metastases, again preferably liver metastases, to a 5-FU- (5-fluorouracil)-containing chemotherapy and/or combination therapy, includes the determination of a possible “responsiveness” or a “response” of a metastatic cell or a metastatic tissue to the therapy. Thus, “responsiveness” represents the induction of a reaction—which promotes the cure of a disease or disorder (in this context, a proliferative disease)—of a single cell, a cell population, a cell aggregate, a tissue, an organ or an organism. The term “responsiveness” comprises particularly the sensitivity of individual (proliferatively altered and/or metastatic) cells, a cell population or a cell aggregate/tissue to 5-FU alone or in combination with other drugs. The terms “responder” and “non-responder” are known to the person skilled in the art and have already been explained above. Patients are evaluated as “responders” if they present a complete remission (CR) or a partial remission (PR); patients who present a stable tumor course (SD, stable disease) or a progressive tumor course (PD, progressive disease) in the imaging are evaluated as “non-responders.” The response to the chemotherapy correlates with the survival of the patients in colorectal carcinoma. Factors that could predict the response to the therapy would lead to a recommendation of the therapy in the case of a “responder,” and to offering the patient existing therapy alternatives in the case of a “nonresponder.”

Here the “prognostic factor” and the “predictive factor” also play a role. In this case, the “prognostic factor” is a variable which has an independent influence on the disease course. Clinical, histopathological and molecular prognostic factors were evaluated in 2000 by the American Joint Committee on Cancer Prognostic Factors (AJCC) in colorectal carcinoma (Compton C. et al., Cancer 88:1739-57 (2000)). An unequivocal prognostic relevance was assigned to the pT category, the pN category, invasion of venous and lymphatic vessels, the residual tumor R classification, and to the preoperative CEA value. To date, the molecular markers are still not considered to have been sufficiently validated.

The “predictive factor” is a variable which has an independent influence on the response of a nonsurgical therapy. Here too, the molecular factors are not yet sufficiently validated for the prediction of a palliative chemotherapy in colorectal carcinoma.

It is preferred for the proliferatively attached (metastatic) cell/the cell aggregate to die and/or to be stopped in its proliferation as a result of the 5-FU therapy. A combination therapy with 5-FU comprises, among other steps, the additional administration to the patients of other drugs, which preferably are also cancer drugs. However, the drugs can also include, for example, immunostimulating drugs. Other drugs that can be used in cancer or chemotherapy are platinum compounds (such as, for example, cisplatin or oxaliplatin). The additional administration of, for example, folic acid is a combination therapy in the sense of this invention. In the examples, it is demonstrated that the invention described here can also be used particularly in the palliative first-line therapy where, in the specific example, 5-FU was used with oxaliplatin. The 5-fluorouracil (5-FU) based chemotherapy represents the most important pillar of the chemotherapy of colorectal carcinoma. Usually, folic acid is added for biomodulation. In the past, 5-FU was administered as a bolus (so-called Mayo regimen), but, in the meantime, studies have become available which show that if the therapy is administered as a 24-h infusion (so-called AIO [Working Group for Internal Oncology] regimen), it is tolerated considerably better, less diarrhea, mucositis and hand-foot syndrome occurs, and that the therapy is also has a better response. Other therapy options consist of treatment with the orally available 5-FU derivative Xeloda® (which is administered without folic acid) or the combination therapy with, for example, folic acid, irinotecan, cisplastin or oxaliplatin. Other combinations which are currently being tested in phase I-III studies are 5-FU-containing schemes in combination with, for example, cetuximab, erlotinib, bevacizumab or vatalanib.

The concept “metastases,” in connection with the above-mentioned method for predicting/determining the response to a therapy, preferably a 5-FU-containing chemotherapy and/or combination therapy, comprises “disseminated (tumor) cells” in the affected patient's body. The concept “metastases” comprises particularly, according to the invention, liver, lung, brain, bone and/or peritoneal metastases, and particularly liver metastases.

In connection with the invention, the concept “gene expression profile” comprises the determination of “expression profiles,” and also of “expression levels” [English] or “expression levels” [French] of the corresponding genes.

The concept “expression level” and the concept “expression profile,” according to the invention, comprise both the quantity of the expression product and the quality of the expression product, for example, products of alternate splicing processes, methylations, glycosylations, phosphorylations, etc. Thus, in the determination of the “expression profile,” in connection with the invention, the focus is essentially on the quantity of the corresponding gene product (RNA/protein). The expression level also takes quantity into account, but, if applicable, it is compared to other tissues, or to tissues and cells from other (preferably healthy) individuals. Optionally, modifications in the gene product (such as, for example, mutations) are also determined in comparison to other tissues. Corresponding embodiment examples are documented in the experimental part, and also particularly in the tables, preferably in Tables A, B and C.

In a particular embodiment, the invention comprises the above-mentioned method where the expression profile of at least two of the 120 marker genes, as represented in SEQ ID NO: 1 to SEQ ID NO: 181, is determined. As mentioned above, the 120 marker genes are also defined by variants, which again are represented in Table D. This expression profile is preferably compared with the expression profile of a “reference.” A reference can be, for example, the expression profile of healthy tissue (for example, intestinal tissue or tissue of the liver, lung, etc.). As “healthy tissue,” one can use here tissue of the affected individual (patient), if that tissue is known not to have undergone any proliferative change, or even be metastatic. Corresponding examples are represented in the experimental part of the invention. However, tissues of other individuals, preferably healthy individuals, can also be used as “reference” or “reference value.”

The determination of the expression profiles is carried out particularly in tissues and/or individual cells of the tissue. Single cell analyses are known in the state of the art and they also comprise DNA or RNA determinations; see, among other references, the detection of individual copies of individual genes as represented in Klein (1999) PNAS 96:4494-4499 or the determination of genomic imbalances as described, for example, in Solinas-Toldo (1997) Genes, Chromosomes, Cancer 20:399-407 genes. It is also possible to determine the expression profile of the genes (gene segments) represented therein by techniques such as in situ hybridization, the determination of the copy number of individual genes (DNA copy number) by comparative genomic hybridization. Other methods for determining the expression profile therefore comprise (in the sense of this invention) the determination by the PCR methodology and the use of biochips (see also the experimental part of the invention). As documented in the experimental part, the determination of the “expression profile” or the “expression level” comprises particularly the quantitative, optionally comparative, determination of the gene expression products, particularly of RNA (but also of proteins). For the determination of the expression profile of the gene (or gene segments) disclosed herein, the following methods can also be used: immunodetection of the proteinaceous gene product (for example, Western blot, ELISA techniques or immunodetection with microscopic analysis); biochemical determinations of the expression product (for example, immunoprecipitations, enzyme tests). In carrying out the invention, it is preferred to not investigate exclusively all the genes mentioned here; however, the best results are achieved if at least 80, preferably at least 70, more preferably at least 60, more preferably at least 50, more preferably at least 40, more preferably at least 30, more preferably at least 30 [sic], more preferably at least 20, more preferably at least 15, more preferably at least 10, more preferably at least 10 [sic], more preferably at least 5, more preferably three, more preferably at least two genes, or their expression profile or expression level are/is determined. The person skilled in the art can without difficulty combine the technical teaching of this invention with the determination of other parameters, particularly other markers, marker genes, in order to be optionally able to make other diagnostic statements. For example, additional tumor markers and/or metastasis markers can be determined. These additional tumor markers are known to the specialists. Tumor markers are, for example, p53, α-fetoprotein, and CEA (carcinoembryonic antigen). CEA is a standard tumor marker for evaluating tumors of the large intestine. As mentioned above, clinical, histopathological and molecular prognostic factors are described and proposed by the American Joint Committee on Cancer Diagnostic Factors (Compton (2000), loc. cit.).

As presented above, according to the invention, in the method for detecting a (colorectal) carcinoma, at least three, but preferably at least two of the marker genes represented here (selected from the group of the 120 gene/gene segments, as shown in SEQ ID NOs: 1-181) are determined. Additional embodiments can be obtained from the experimental part.

The methods, mentioned above under secondary point (a), for detecting a (colorectal) carcinoma can comprise particularly the selection and the determination of the expression profile of at least one, preferably at least two, and most preferably at least three genes of the 120 marker genes as shown in SEQ ID NO: 1 to SEQ ID NO: 181, where the expression profile of the gene is compared with the average expression profile of normal intestinal mucosa. As already mentioned above, the normal intestinal mucosa can originate from the patient, or also from other individuals.

In the following embodiments, preferred genes which can be used for detecting a colorectal carcinoma in the method according to the invention are represented.

Thus, for detecting a colorectal carcinoma, the method comprises particularly the determination of the gene expression profile of at least two genes, where at least one of the two genes (as represented in SEQ ID NOs: 1-120) from the “minimal sets” or “subsets,” as defined below, is used. These “minimal sets” comprise preferably the corresponding above-mentioned and selected 72, 55, 33, 18, 17 or 3 genes. From the “minimal sets”/gene panels/“subsets” defined herein, individual genes/gene expression profiles can be determined according to the invention for the in vitro detection of a carcinoma, preferably a colorectal carcinoma. Additional details can also be obtained from the experimental part and the tables. As discovered here unexpectedly, for example, a particularly preferred “minimal set” for determination/prediction of a colorectal carcinoma comprises the 3 marker genes as characterized in SEQ ID NOS: 25, 41 and 68. Variants of the 3 marker genes are represented in SEQ ID NOS: 136-138.

In this, as well as in the following embodiments, it is understandable to the person skilled in the art that they are preferred embodiments. The person skilled in the art will also determine only individual genes, preferably at least two, more preferably at least 3 genes (or their gene expression profiles/gene expression levels) according to the invention. In the process, he will also use/be able to use a selection of the different “minimal sets” represented here.

In a preferred embodiment of the method comprising the invention for detecting a (colorectal) carcinoma, a selection is made of the marker genes of the expression profile of at least one gene, preferably at least two genes, preferably at least 3 genes, of the 72 marker genes, which are represented in SEQ ID NO: 1 to SEQ ID NO: 72 or SEQ ID NO: 121 to SEQ ID NO: 156, and evaluated. Again one can, for example (and preferably), determine the expression profile of these 72 genes (or a selection thereof) in the tissue to be tested and compare it with healthy tissue. Corresponding embodiments and tissue details can be obtained from the experimental part.

In a more preferred embodiment, the invention comprises a method for detecting a (colorectal) carcinoma where the expression profile of at least one gene, preferably at least two genes, more preferably of at least three genes of 55 marker genes is determined. These 55 genes are defined in Table D, particularly via the SEQ ID NOs: 10, 14, 25, 41, 52, 63, 68 and SEQ ID NO: 73 to SEQ ID NO: 120, but they also comprise variants of these genes, as represented in SEQ ID NOs: 136, 137, 138, 153, 154, 155, 157-181.

In a more preferred embodiment, the invention comprises a method for detecting a (colorectal) carcinoma where the expression profile of at least one gene, preferably at least two genes, more preferably of at least three genes of the 33 marker genes is determined. These 33 genes are defined in Table D particularly via the SEQ ID NOs: 2, 7, 8, 12, 19, 21, 25, 33, 38, 41, 45, 49, 50, 51, 54, 66, 68, 70, 78, 83, 85, 86, 92, 99, 101, 103, 104, 105, 109, 112, 114, 115 and 118, but also genes variants of these genes, as represented in SEQ ID NOs: 122, 124, 136-140, 157, 159-162, 170, 171, 172, 174 and 177-180.

In a more preferred embodiment, the invention comprises a method for detecting a (colorectal) carcinoma where the expression profile of at least one gene, preferably at least two genes, more preferably of at least three genes of the 18 marker genes is determined. These 18 marker genes are defined in Table D particularly via the SEQ ID NOs: 2, 7, 8, 12, 19, 21, 25, 33, 38, 41, 45, 49, 50, 51, 54, 66, 68 and 70, but also variants of these genes, as represented in SEQ ID NOs: 122, 124 and 136-140. Another selection of the at least 18 genes which is also according to the invention comprises in this embodiment the determination of at least one gene, preferably at least two genes, more preferably at least three genes of the 18 marker genes which comprise the genes, as represented in SEQ ID NOs: 25, 41, 68, 78, 83, 85, 86, 92, 99, 101, 103, 104, 105, 109, 112, 114, 115 or 118, but also variants of these genes, as represented in SEQ ID NOs: 136-138, 157, 159-162, 170-172, 174 and 177-180.

In a most preferred embodiment in the sense of the invention, the invention comprises a method for detecting a (colorectal) carcinoma where the expression profile(s) of at least one, preferably at least two, and more preferably all three of the 3 marker genes is/are determined. These 3 genes are defined in Table D, particularly via the SEQ ID NOs: 25, 41 and 68, but they also comprise variants of these genes as represented in SEQ ID NOs: 136-138, and they also comprise additional variants and homologs of these genes.

As defined below, the term “marker genes” in the sense of this invention comprises not only the specific gene sequences (or the corresponding gene products), as represented in the specific nucleotide sequences, but also gene sequences which are homologous, preferably highly homologous, with these sequences. “Highly homologous sequences” comprise sequences which present at least 80%, preferably at least 90%, most preferably at least 95% homology with the sequences represented in the SEQ ID NOS:1-181. In the context of this invention, “highly homologous sequences” also comprise sequences which code for gene products (for example, RNA or proteins), which are at least 80% identical to the defined gene products of SEQ ID NOS: 1-181.

The sequence identity can be determined in the usual manner using computer programs such as, for example, the Bestfit program (Wisconsin Sequence Analysis Package, Version 8 for Unix Genetics Computer Group, University Research Park, 575 Science Drive Madison, Wis. 53711). Bestfit uses the local homology algorithm of Smith and Waterman, Advances in Applied Mathematics 2 (1981) 482-489, to find the segment with the highest sequence identity between two sequences. When using Bestfit or another sequence alignment program for determining whether a certain sequence is, for example, 95% identical to a reference sequence of the present invention, the parameters are preferably set so that the percentage of identity is calculated over the entire length of the reference sequence, and homology gaps (“gaps”) of up to 5% of the total number of nucleotides in the reference sequence are allowed. When using Bestfit, the so-called optional parameters are preferably left at the preset (“default”) values. The deviations which occur in the comparison of a given sequence with the above-described sequences of the invention can be caused, for example, by addition, deletion, substitution, insertion or recombination. It is preferred for such a sequence comparison to be carried out additionally with the program DNASIS (Version 6.0, Hitachi Software Engineering co. Ltd., 1984, 1990). Here too, the “default” parameter settings (cut-off score: 16, Ktup: 6) should be used.

The data of the experimental part show that by determining at least one, preferably two, or most preferably three marker genes as defined via Table D and SEQ ID NOS: 25, 41 and 68 (and also represented by the variants as represented in SEQ ID NOS: 136-138), a clear determination of colorectal carcinoma can be carried out; see also Examples 1-3 and 17.

The method already mentioned under secondary point (b) for predicting metastases, preferably liver metastases dependent on a primary (colorectal) carcinoma can comprise particularly the selection and the determination of the expression profile of at least one gene, preferably at least two genes of the 120 marker genes disclosed here, where these genes are defined via SEQ ID NO: 1 to SEQ ID NO: 181, and where the expression profile of the genes which is obtained from potential metastatic cells or tissues, preferably in liver cells or liver tissue, is compared with the expression profile obtained from primary colorectal tumor cells or tissues. The samples for the obtention of the two expression profiles can originate from the same patient, or also from different patients.

In the following embodiments, preferred genes, which can be used in the inventive method for predicting metastases, preferably liver metastases dependent on a primary (colorectal) carcinoma are represented. Thus the method comprises particularly the determination of the gene profile of at least one, preferably at least two genes, where at least one of the two genes of the 120 marker genes (as represented in SEQ ID NOs: 1-181) from the “minimal sets,” as defined below, is used. These “minimal sets” comprise, among other selections and preferably, the 72, 55, 22, 20 or 2 genes mentioned in connection with this embodiment. However, as in other embodiments, it is also possible to construct other “minimal sets”/“subsets” or gene panels, where the ones mentioned here are preferred. As one can see in Tables 2 and 3, in the determination of the expression data, not all 120 genes are absolutely purely indicative (marked “none”). However, these genes or their expression profile can also be determined according to the invention, and the corresponding results can be used, among other purposes, as internal controls. Additional details can also be obtained from the experimental part, and from the tables (Tables 2 and 3).

In a preferred embodiment of the method comprising the invention for predicting metastases dependent on a primary (colorectal) carcinoma, preferably liver metastases, a selection of the marker genes of the expression profile of at least one gene of the 72 marker genes defined in Table 2 is made, which marker genes are also represented in SEQ ID NO: 1 to SEQ ID NO: 72 or SEQ ID NO: 121 to SEQ ID NO: 156, and evaluated. Again, for example (and preferably), the expression profile of the 72 genes can be obtained from potential metastatic cells or tissues, preferably liver cells or liver tissues, and compared with the expression profile from primary colorectal tumor cells or tissues.

In a more preferred embodiment, the invention comprises a method for predicting metastases dependent on a primary carcinoma, particularly a colorectal carcinoma, preferably predicting liver metastases, where the expression profile of at least one gene of the 55 marker genes defined in Table 3 is determined. These 55 genes are defined in Table D, particularly via SEQ ID NOs: 10, 14, 25, 41, 52, 63, 68 and SEQ ID NO: 73 to SEQ ID NO: 120, however, they also comprise variants of these genes as represented in SEQ ID NOs: 136, 137, 138, 153, 154, 155, 157-181. In this embodiment, as well as the following embodiments, it is clear to the person skilled in the art that they are preferred embodiments. The person skilled in the art will also, according to the invention, determine only individual genes, preferably at least two, more preferably at least 3 genes (or their gene expression profiles/gene expression levels). In the process, he will also use/be able to use a selection of the different “minimal sets” or gene panels represented here, where these “minimal sets” already represent a selection of the 120 marker genes according to the invention. In a more preferred embodiment, the invention comprises a method for predicting metastases dependent on a primary carcinoma, particularly a colorectal carcinoma, preferably predicting liver metastases, where the expression profile(s) of at least one gene, preferably at least two, more preferably at least 3, more preferably at least 5 of the 22 marker genes defined in Table C is/are determined. These 22 genes are defined in Table D, particularly via the SEQ ID NOs: 17, 19, 20, 22, 26, 29, 30, 31, 33, 35, 37, 40, 48, 58, 59, 64, 66, 69, 71, 72, 74 and 109, but they also comprise variants of these genes as represented in SEQ ID NOs: 125, 133, 134, 135, 151, 152, 156 and 177.

In a more preferred embodiment, the invention comprises a method for predicting metastases dependent on a primary carcinoma, particularly a colorectal carcinoma, preferably predicting liver metastases where the expression profile of at least one gene, preferably at least two genes, more preferably at least three genes, more preferably at least 5 genes of the 20 marker genes is determined. These 20 genes are defined in Table D, particularly via SEQ ID NOs: 17, 19, 20, 22, 26, 29, 30, 31, 33, 35, 37, 40, 48, 58, 59, 64, 66, 69, 71 and 72, but they also comprise variants of these genes as represented in SEQ ID NOs: 125, 133, 134, 135, 151, 152 and 156.

In a most preferred embodiment in the sense of the invention, the invention relates to a method for predicting metastases dependent on a primary (colorectal) carcinoma, preferably liver metastases where the expression profile of at least one of the two marker genes as represented in SEQ ID NOs: 74 and 109, is determined.

The prediction of metastases of a primary colorectal carcinoma is particularly important for children, because it can influence the further treatment of the patient. If no metastases are found (neither lymph node metastases nor distant metastases), the stage is called UICC stage I or II. These tumors, if they are colon carcinomas, are then treated by surgery. An additional adjuvant chemotherapy is not provided for (outside of the clinical studies). However, if lymph nodes metastases (UICC stage III) are found, then, according to the guideline of the German Cancer Association and other international associations, a postoperative adjuvant chemotherapy is required. Adjuvant chemotherapy results in 3-year disease-free survival rate for the patients of approximately 80%, whereas without subsequent chemotherapy the 3-year survival rate is only approximately 60%. The overall survival is significantly influenced by the adjuvant chemotherapy. In the case of rectal carcinoma, it is also of decisive importance whether lymph node metastases are already present. In these cases, preoperative radiochemotherapy is recommended because it significantly reduces the development of local recurrences in the rectum. In addition, preoperative radiochemotherapy results in a significantly greater number of patients being operated on under continence-preserving conditions, which, in these patients, leads to a considerable improvement of the postoperative quality of life.

If distant metastases are already present in a patient with colorectal carcinoma (UICC stage IV), then, after a curative operation of the primary tumor, a palliative chemotherapy should be carried out to extend the survival time of the patients. In addition, by using palliative therapy, up to 15% of the patients who were considered inoperable because of distant metastases can receive secondary curative surgery, and they have an overall 5-year survival rate of approximately 30%.

The imaging procedures that are usually used in the diagnosis and therapy planning of colorectal carcinomas (for example, computer tomography (CT), magnetic resonance tomography (MRT), positron emission tomography (PET) or preoperative endosnography [sic; endosonography] in rectal carcinomas) are frequently unable to detect small aggregates of tumor cells (so-called micrometastases) in lymph nodes or in other organs. Therefore, the actual tumor stage is often evaluated at too low a level, and an adjuvant or palliative therapy that may have been required is not administered. Therefore, a particularly high clinical relevance is associated, as disclosed in the invention, with the molecular prediction of metastases dependent on a primary colorectal carcinoma, that is the prediction of individual tumor cells that have already established separate colonies from the primary tumor, while not yet presenting clinically symptomatic metastases, in distant regions of the body, usually the lymph nodes and liver, because molecular prediction leads to therapeutic decisions which potentially increase the life and the quality of life of patients with colorectal carcinomas. Surprisingly, the markers disclosed in this invention are also suitable for the early detection of metastases and their precursors. In addition, a preferred embodiment, which consists of measuring proteins or protein fragments and peptides of the translated gene sequences of the 120 genes in the serum or plasma by simple blood collection the context of the usual standard diagnosis can be easily integrated and does not require any additional, invasive interventions while at the same time providing useful information with regard to the therapy management. This capacity highlights the technical and diagnostic superiority of the in vitro methods and the corresponding diagnostic kits which are disclosed herein, compared to the methods that have been standard to date.

The method, mentioned above under secondary point (c) for predicting the response to a therapy, particularly the response of metastases to a 5-FU-containing chemotherapy or combination therapy, can comprise particularly the selection and the determination of the expression profile of at least one, preferably at least two genes, more preferably at least three genes from the group of the 120 marker genes disclosed here, where these genes are defined via the SEQ ID NO: 1 to SEQ ID NO: 181, and where the expression profile of the genes, obtained from tumor or metastasis samples, preferably metastasis samples of patients who respond to a therapy, is compared with the expression profile from metastasis samples of patients who do not respond to a therapy. The corresponding therapy is a 5-FU-containing chemotherapy and/or a 5-FU-containing combination therapy.

However, the corresponding marker genes can be determined not only in tumor and/or metastasis samples, but also in other biological products such as, for example, blood or blood serum. As explained below, it is possible to determine, for example, “acute phase” markers/“acute phase” proteins/“acute phase” genes in blood/blood serum (or optionally other body fluids such as axillary ascites or lymph fluid), to distinguish “responders” from “non-responders.”

In the following embodiments, the preferred genes are represented which can be used in the method according to the invention for predicting the response of metastases to a therapy. Thus, the method comprises particularly the determination of the gene expression profile of at least one, preferably two genes, where one uses at least one of the two genes of the 120 marker genes (as represented in SEQ ID NOs: 1-181) from the “minimal sets,” as defined below. These “minimal sets” comprise, among other genes and preferably, the mentioned 72, 55, 34, 28 or 7 genes. However, as needed, other “minimal sets”/gene panels selected from the 120 marker genes represented here can be constructed, which lead to the determination of “responders” and/or “non-responders.” For example, it is preferred to select a “minimal set”/subset/gene panel of the 120 marker genes defined herein, whose determination in biological fluids, specimens (such as, for example, and particularly blood/blood serum, or also lymphatic fluid) is simplified. Such a “minimal set”/subset/gene panel comprises, for example, the “acute phase proteins/genes” disclosed herein.

Therefore, the invention comprises the determination of serum markers, in an embodiment, the determination and/or prediction, differentiation of “responders”/“non-responders” to a 5-FU-containing chemotherapy and/or combination therapy. In a preferred embodiment of this invention, for differentiating responders from non-responders to a 5-FU-containing chemotherapy or combination therapy, it is suitable to use the determination of serum markers from blood or other body fluids, as well as from the stool of the patients, particularly before chemotherapy. Mention should be made here particularly of the so-called acute phase proteins, which are already used in daily clinical practice to observe the course of inflammations under antibiotic therapy, and which have been shown to be robust and valid parameters in infection wards. Surprisingly, these markers are also suitable for predicting the response of primary tumors and metastases to chemotherapy. The genes of the underlying acute phase proteins—which are also listed in Table 1—are: complement component 1, q subcomponent, beta polypeptide [Affymetrix number 202953_at], SEQ ID number 4; apolipoprotein E [Affymetrix number 203382_s_at], SEQ ID number 6; apolipoprotein C-I [Affymetrix number 204416_x_at], SEQ ID number 13; coagulation factor V (proaccelerin, labile factor) [Affymetrix number 204714_s_at], SEQ ID number 18; fibrinogen, B beta polypeptide [Affymetrix number 204988_at], SEQ ID number 20; orosomucoid 1 [Affymetrix number 205041_s_at], SEQ ID number 22; apolipoprotein B (including Ag(x) antigen) [Affymetrix number 205108_s_at], SEQ ID number 23; fibrinogen, A alpha polypeptide [Affymetrix number 205650_s_at], SEQ ID number 29; coagulation factor II (thrombin) [Affymetrix number 205754_at], SEQ ID number 30; transferrin [Affymetrix number 214063_s_at], SEQ ID number 58; complement component 4A [Affymetrix number 214428_x_at], SEQ ID number 59; serum amyloid A2 [Affymetrix number 214456_x_at], SEQ ID number 60; orosomucoid 2 [Affymetrix number 214465_at], SEQ ID number 61; complement component 3 [Affymetrix number 217767_at], SEQ ID number 66; complement component 1, q subcomponent, alpha polypeptide [Affymetrix number 218232_at], SEQ ID number 67; fibrinogen, gamma polypeptide [Affymetrix number 219612_s_at], SEQ ID number 69; and C-reactive protein, pentraxin-related [Affymetrix number 37020_at], SEQ ID number 71.

The above-mentioned genes/gene products thus represent another “minimal set”/gene panel or “subset” of the invention. It is preferred for the expression profile to consist of at least one, more preferably at least 3, more preferably at least 5, most preferably at least 7 of the above-mentioned “acute phase” genes (or gene products) for predicting “responders” and “non-responders” to a 5-FU-containing therapy. Additional details can be obtained from the experimental part and the tables.

In a preferred embodiment of the method of the invention for predicting the response of metastases to a 5-FU-containing therapy, a selection is made of the expression profiles from the 72 marker genes defined in Table 1, which are represented in the SEQ ID NO: 1 to SEQ ID NO: 72 or SEQ ID NO: 121 to SEQ ID NO: 156, and evaluated. Again, for example (and preferably), the expression profile of the genes obtained from metastasis samples of patients who respond to a therapy is compared to the expression profile from metastasis samples of patients who do not respond to a therapy.

In a more preferred embodiment, the invention comprises a method for predicting the response of metastases to a therapy, particularly to a 5-FU-containing therapy, where the expression profile of at least one gene of the 55 marker genes defined in Table 4 is determined. These 55 genes are defined in Table D, particularly via the SEQ ID NOs: 10, 14, 25, 41, 52, 63, 68 and SEQ ID NO: 73 to SEQ ID NO: 120, but they also comprise variants of these genes, as represented in SEQ ID NOs: 136, 137, 138, 153, 154, 155, 157-181. In this embodiment as well as in the following embodiments, the person skilled in the art knows that these are preferred embodiments. The person skilled in the art will also determine only individual genes, preferably at least two, more preferably at least 3 genes (or their expression profiles/gene expression levels). These genes can originate from different “minimal sets” defined here.

In a more preferred embodiment, the invention comprises a method for predicting the response of metastases to a therapy, where one determines the expression profile of at least one, preferably at least two, more preferably at least 3, more preferably at least 5 gene(s) of the 34 marker genes defined in Table A. These 34 genes are defined in Table D, particularly via the SEQ ID NOs: 6, 7, 8, 10, 13, 14, 24, 25, 41, 45, 46, 48, 52, 54, 60, 61, 63, 65, 66, 68, 73, 78, 79, 80, 89, 97, 98, 101, 104, 107, 108, 116, 117 and 120, but they also comprise variants of these genes as represented in SEQ ID NOs: 122, 136-147, 151-155, 157, 167, 168, 169, 171, 172, 176 and 181.

In a more preferred embodiment, the invention comprises a method for predicting the response of metastases to a therapy, where one determines the expression profile of at least one, preferably at least two, more preferably at least 3, more preferably at least 5 gene(s) of the 28 marker genes defined in Table A. These 28 genes are defined in Table D, particularly via the SEQ ID NOs: 6, 7, 8, 13, 24, 45, 46, 48, 52, 54, 60, 61, 65, 66, 73, 78, 79, 80, 89, 97, 98, 101, 104, 107, 108, 116, 117 and 120, but they also comprise variants of these genes, as represented in SEQ ID NOs: 122, 139-147, 151, 152, 157, 167, 168, 169, 171, 172, 176 and 181.

In a most preferred embodiment in the sense of the invention, the invention comprises a method for predicting the response of metastases to a therapy where the expression profile of at least one, preferably at least two, more preferably at least 3 of the following 7 marker genes is determined. These 7 genes are defined in Table D, particularly via the SEQ ID NOs: 10, 14, 25, 41, 52, 63 and 68, but they also comprise variants of these genes as represented in SEQ ID NOs: 136, 137, 138, 152, 154 and 155.

The term “marker gene” in connection with this invention comprises, according to the invention; a gene or a gene segment which presents at least 60% homology, preferably at least 65% homology, more preferably at least 70% homology, more preferably at least 75% homology, more preferably at least 80% homology, more preferably at least 85% homology, more preferably at least 90% homology, more preferably at least 95% homology, more preferably at least 96% homology, more preferably at least 97% homology, more preferably at least 98% homology, more preferably at least 99% homology, and most preferably at least 100% homology with the gene represented in SEQ ID NO 1 to SEQ ID NOs 181, in the form of deoxyribonucleotides or corresponding ribonucleotides, or the proteins derived therefrom. A protein derived from the 120 marker genes (defined in SEQ ID NOs: 1-181, for example, in Table D), denotes in this invention, according to this invention, a protein, a protein fragment or a polypeptide which is translated in the native reading frame (in frame).

The “obtention of an expression profile” of the marker genes comprises, according to the invention, the obtention of the above-described expression profile from one or more samples of one or more individuals.

These “individuals” are primarily humans, however, the invention can also be applied to other mammals, and particularly to apes, mice, rats, pigs, horses, dogs, cats, hares, rabbits, hamsters and guinea pigs. Moreover, the term “individual” comprises both sick and also healthy representatives of the above-mentioned species.

A “sample,” according to the invention, denotes one or more cells, tissues, a complete organ or a part thereof. Tumor tissues/tumor cells/metastatic tissue/metastatic cells can also be “samples.” In a preferred embodiment of the invention, the sample is selected from the group consisting of a surgery preparation, tissue biopsy, peritoneal fluid, blood, serum, plasma, lymphatic fluid, lymphatic tissue, urine and stool. As defined here and also represented in the experimental part, the in vitro method presented herein can also be carried out on biological fluids, among other substances, such as blood and/or blood serum. The “samples” to be analyzed are not limited to freshly collected samples, rather, the samples can also comprise fixed or frozen material.

Accordingly, the present invention is not limited to the investigation of fresh material, and the results according to the invention can also be obtained by examining fixed material, for example, paraffin material. Thus, the results represented here have been successfully transferred from fresh colorectal tissue to paraffinized colorectal tissue, although the two materials present considerable differences, for example, primary tumor tissue instead of metastasis tissue, fixed tissue instead of fresh tissue, mixed tissue instead of microdissociated tumor cells. At the same time, it was possible to show that the methods represented here can be reproduced not only with gene chips, but also by other methods, such as, for example, RT-PCR. In fixed material in particular, it is preferred to also use other detection methods for detecting the gene/gene expression products, for example, RNA specific primers and probes at exon-exon boundaries within the gene, instead of probes against a 3′-region of the genes. In the clinical situation, the investigative material present is usually fixed (i.e., FFPE [formalin-fixed paraffin-embedded] tissue of the primary tumor derived from the tumor resection before chemotherapy). Therefore, the person skilled in the art, according to the invention, will tend to use this detection method. The tests on fixed materials confirm impressively the validity of the (marker) genes and “minimal sets”/“subsets” presented and defined here, both in the in vitro diagnosis of colorectal carcinoma, and also (in a completely different experimental setup) a separation of responders and non-responders to the 5-FU-containing therapy. In addition, several of the primary tumors investigated were not included in the original finding (fresh tissue samples from the metastases), and can thus be considered an independent validation of the significance of the marker genes. To confirm the significance of the 120 marker genes (and marker gene products) described here, RNA was extracted from paraffin tissues, which in part were older than 4 years, originating from primary tumors and metastases, and a gene set of 6 genes from the 120 marker genes as defined herein was analyzed. Differences in the gene expression between “responders” and “non-responders” in the RNA of the paraffin-embedded tumors (colorectal carcinoma, lymph node metastases, peritoneal carcinosis or distant metastases (for example, liver, lung)), were confirmed for the genes: Orosomucoid 1 [Affymetrix number 205041_s_at], SEQ ID number 22; peroxisome proliferative activated receptor, gamma [Affymetrix number 208510_s_at], SEQ ID number 41; homeobox DlI [Affymetrix number 214604_at], SEQ ID number 62; mitogen-activated protein kinase kinase kinase 5 [Affymetrix number 203837_at], SEQ ID number 81; spondin 1 (f-spondin) extracellular matrix protein [Affymetrix number 209436_at], SEQ ID number 98; and homeobox A9 [Affymetrix number 209905_at], SEQ ID number 99. These data are in agreement with the results which are obtained with fresh material. Corresponding shifts in the gene expression in the comparison of “responders” and “non-responders” can be read/determined particularly also in Tables 1 and 4. It is worthy of note here that a gene/a gene expression which, in the comparison between non-responders and responders, is upregulated, is marked “up,” while a gene/a gene expression which, in a comparison between non-responders and responders, is downregulated, is marked “down.” In Table 5—also in the appendix—similar data are presented and data are compared which originate from a specific patient (Patient T). In Table 5; column 6 (in contrast to Tables 1 and 4), the change in the expression profile/level in responders (compared to nonresponse/non-responders) is represented. Thus, for example, the gene profile/gene expression level of the gene characterized by SEQ ID NO: 4 is marked “up” in Table 1 (the gene expression is higher in “non-responders” than in “responders”), and in Table 5 with “↓,” because in Table 5 “responders” are compared to “non-responders.” Thus, Table 5 shows that the gene has a lower expression level in “responders” than in “non-responders.”

As experimentally shown, it is possible to use, particularly in a preferred embodiment of the invention for differentiating between “responders” or “non-responders” to a 5-FU-containing chemotherapy/combination therapy, a “minimal set” of the 120 marker genes defined herein. A corresponding “minimal set”/subset comprises, for example, the following genes/gene markers: complement component 1, q subcomponent, beta polypeptide [Affymetrix number 20953_at], SEQ ID Number 4; apolipoprotein E [Affymetrix number 203382_s_at], SEQ ID number 6; apolipoprotein C-I [Affymetrix number 204416_x_at], SEQ ID number 13; coagulation factor V (proaccelerin, labile factor [Affymetrix number 204714_s_at], SEQ ID number 18; fibrinogen, B beta polypeptide [Affymetrix number 204988_at], SEQ ID number 20; orosomucoid 1 [Affymetrix number 205041_s_at], SEQ ID number 22; apolipoprotein B (including Ag(x) antigen) [Affymetrix number 205108_s_at], SEQ ID number 23; fibrinogen A alpha polypeptide [Affymetrix number 205650_s_at], SEQ ID number 29; coagulation factor II (thrombin) [Affymetrix number 205754_at], SEQ ID number 30; transferrin [Affymetrix number 214063_s_at], SEQ ID number 58; complement component 4A [Affymetrix number 214428_x_at], SEQ ID number 59; serum amyloid A2 [Affymetrix number 214456_x_at], SEQ ID number 60; orosomucoid 2 [Affymetrix number 214465_at], SEQ ID number 61; complement component 3 [Affymetrix number 217767_at], SEQ ID number 66; complement component 1, q subcomponent, alpha polypeptide [Affymetrix number 218232_at], SEQ ID number 67; fibrinogen, gamma polypeptide [Affymetrix number 219612_s_at], SEQ ID number 69; and C-reactive protein, pentraxin-related [Affymetrix number 37020_at], SEQ ID number 71.

If necessary, the person skilled in the art will again analyze from this subset only individual genes (gene products), preferably at least two, more preferably at least three, more preferably at least five genes (or gene products) for the determination of “responders” or “non-responders.”

The Tables 1, 4 and 5 in the appendix particularly provide information regarding the changes of the expression profile of individual genes in the comparison between “responders” and “non-responders”/“non-response.” Thus, for example, for the above-mentioned genes, in the comparison of “responder” versus “non-responder” according to Table 5, the following gene expression qualities are indicated (where “down” means that the corresponding gene expression is downregulated in “responders” in comparison to “non-responders”):
Complement component 1, q subcomponent, beta polypeptide [Affymetrix number 202953_at], SEQ ID number 4: down
Apolipoprotein E [Affymetrix number 203382_s_at], SEQ ID number 6: down
Apolipoprotein C-I [Affymetrix number 204416_x_at], SEQ ID number 13: down
Coagulation factor V (proaccelerin, labile factor [Affymetrix number 204714_s_at], SEQ ID number 18: down
Fibrinogen, B beta polypeptide [Affymetrix number 204988_at], SEQ ID number 20: down
Orosomucoid 1 [Affymetrix number 205041_s_at], SEQ ID number 22: down
Apolipoprotein B (including Ag(x) antigen) [Affymetrix number 205108_s_at], SEQ ID number 23: down
Fibrinogen A alpha polypeptide [Affymetrix number 205650_s_at], SEQ ID number 29: down
Coagulation factor II (thrombin) [Affymetrix number 205754_at], SEQ ID number 30: down
Transferrin [Affymetrix number 214063 s_at], SEQ ID number 58: down
Complement component 4A [Affymetrix number 214428_x_at], SEQ ID number 59: down
Serum amyloid A2 [Affymetrix number 214456_x_at], SEQ ID number 60: down
Orosomucoid 2 [Affymetrix number 214465_at], SEQ ID number 61: down
Complement component 3 [Affymetrix number 217767_at], SEQ ID number 66: down
Complement component 1, q subcomponent, alpha polypeptide [Affymetrix number 218232_at], SEQ ID number 67: down
Fibrinogen, gamma polypeptide [Affymetrix number 219612_at], SEQ ID number 69: down
C-reactive protein, pentraxin-related [Affymetrix number 37020_at], SEQ ID number 71: down

Thus, the serum markers according to Table 5 are then indicative for a “5-FU responder” if they are downregulated, in contrast to “non-responders.”

The above-mentioned serum markers are therefore particularly well suited to analyze by the investigation of body fluids, particularly blood and/or blood serum, the success or the potential success of a therapy, particularly of a 5-FU-containing chemotherapy/combination therapy. As represented above, the gene expression profile (the gene product quantity) is downregulated (“down;” weaker expression) in responders compared to non-responders, see also Table 5. In connection with Table 5, “↓” denotes a weaker expression of the corresponding gene in “responders” versus “non-responders.”

In this embodiment as well, the person skilled in the art will not investigate all of the genes/gene products/gene expressions represented here, rather he will use for the in vitro diagnosis a “minimal set”/“subset” comprising at least 2, preferably at least 3, more preferably at least 5 of the marker genes represented here.

In all the in vitro methods presented here, the person skilled in the art will investigate, according to the invention, at least 1, preferably at least 2, more preferably at least 3, and even more preferably at least 5 of the 120 marker genes represented here for their expression profile (expression level). A positive finding exists (colon carcinoma, prediction of a metastasis or response to a 5-FU-containing chemotherapy/combination therapy (particularly the response of a metastasis)), if at least 60%, more preferably at least 70%, more preferably 80%, preferably at least 90%, more preferably at least 95%, more preferably at least 96%, and most preferably at least 97% of the marker genes investigated present a modification compared to healthy tissue (in the detection of colon carcinoma or a metastasis) or compared to sample material from patients who do not react/respond to a 5-FU-containing therapy (prediction of the response of tumors/metastases to a 5-FU-containing chemotherapy/combination therapy). Corresponding data can also be taken from the experimental part and from the tables to which reference is made herewith. If only one (marker) gene of the selection presented here of the 120 marker genes will be investigated, then the change with respect to the control tissue/control material should be 100%, to yield a positive finding. However, in a particularly preferred method according to the present invention, at least two, preferably at least 3, more preferably at least 5, more preferably at least [sic; omission in source text] gene expression profiles (and thus gene products of the individual marker genes) are analyzed. For example, if 5 genes (or their expression profile) from the 120 marker genes represented here are investigated, then preferably 3 of these 5 genes (60%), more preferably 4 of these 5 genes (80%), more preferably all 5 genes (100%) should present the same expression profile as the corresponding controls (see also the tables in the appendix) to be “positive.”

Table 5 in the appendix also shows, as a nonlimiting example, that for the methods represented here, it is possible to make more than diagnostic statements if all the selected marker genes have the changes presented in the table at a 100% level. For example, patient T is determined according to the invention as a 5-FU “responder,” in spite of the fact that there were deviations in individual marker genes with regard to the gene expression profiles, as in the standard (see Table 5, column 6). Individual, minimal changes are marked in Table 5 in bold print.

As also represented in the experimental part of the invention, the expression profile of the 120 marker genes disclosed here (or a selection thereof) is determined preferably by the measurement of the quantity of marker gene mRNA. This quantity of marker gene mRNA can be determined, for example, by the gene chip technology, (RT-) PCR (for example, on fixed material), Northern hybridization, dot blotting or in situ hybridization. However, the method according to the invention can also be carried out if the person skilled in the art measures and identifies the gene products (if present at the protein or peptide level). Thus, the invention comprises the methods described here in which the gene expression products are determined in the form of their synthesized proteins (or peptides). Here both the quantity and the quality (for example, modifications such as phosphorylations or glycosylations) are determined. In this connection, however, it is preferred for the expression profile of the marker genes to be determined by measuring the polypeptide quantity of the marker genes, and, if desired, comparing them with a reference value. The reference value has already been described above and it can originate from healthy or sick tissue. The polypeptide level or the polypeptide quantity of the marker genes, can be determined by the ELISA, RIA, (immuno-) blotting, FACS, or immunohistochemical methods. In particular, the determination of serum markers, as defined here, can also be made with protein biochemistry methods. For example, the expression pattern of the “acute phase proteins” defined here, as selected from the 120 marker genes disclosed herein, can be carried out via such (protein) biochemistry methods, for example, by ELISA, RIA, immunoblotting, etc. As represented here, such methods are useful particularly in the detection of “responders” and “non-responders” to a chemotherapy and/or combination therapy, particularly a 5-FU-containing therapy.

The invention also relates to a method which comprises the following steps:

(a) determination of the expression profile, as defined above, in a patient sample; and

(b) determination with the help of the gene expression profile established in (a), whether the patient

i. suffers from a colorectal carcinoma;

ii. presents liver metastases subsequent to a colorectal carcinoma; and/or

iii. responds to a 5-FU-containing chemotherapy.

The above-mentioned embodiments also apply to the method represented here. The method will also make available the above-mentioned method to determine whether the patient suffers from a carcinoma, preferably an adenocarcinoma. The method, moreover, is not limited to determining liver metastases or to the response of metastases to a 5-FU-containing therapy. The same applied, mutatis mutandis, to the following methods according to the invention.

The invention also relates to a kit for carrying out the methods described here, where the kit comprises specific (nucleotide) probes, primer (pairs), antibodies, aptamers for the determination of at least two of the 120 marker genes which are represented in SEQ ID NO: 1-181, or for the determination of at least two gene products of the 120 marker genes comprising the marker genes coded in SEC ID NO: 1 to 181. The kit is preferably a diagnostic kit. In the meaning used according to the invention, “kit” also denotes a “microarray” or a “gene chip.”

Thus, the invention also comprises the use of one of the 120 marker genes or a group (particularly the “minimal sets” defined herein or also a selection of these genes) of the 120 marker genes, which are represented in SEQ ID NO: 1-81 to determine whether the patient

(a) suffers from a colorectal carcinoma;

(b) presents liver metastases subsequent to a colorectal carcinoma; and/or

(c) responds to a 5-FU-containing chemotherapy, particularly to determine whether the metastasized tumor cells (metastases) respond to such a therapy.

An additional possible use of the invention can consist of the production of a transgenic, nonhuman animal, preferably a transgenic mouse which overexpresses or underexpresses one or more of the markers. Such a transgenic mouse can be used to prepare a model system, for example for liver metastasis in humans. Thus, the metastasis process, which to date is still only partially understood, could be elucidated, and new knowledge on the metastasizing pathway of colorectal carcinoma or also other carcinomas could be gained. In addition therapeutic or toxic substances could be tested with the help of such a transgenic mouse. A similar principle also constitutes the foundation of knock-out analyses, which can be carried out with the help of the genes of this invention. Thus, the animal according to the invention can also be used for screening procedures.

Therefore, the invention also relates to the use of a transgenic non-human animal which overexpresses or underexpresses one or more of the marker genes defined here in a screening procedure for drugs. This screening procedure is particularly suitable to screen for agents used in cancer therapy, particularly in the treatment of colorectal carcinomas and/or metastases, preferably liver metastases.

The use of a transgenic cell which overexpresses or underexpresses one or more of the marker genes defined here in a screening procedure for drugs is also a part of the invention. Here too, it is preferred to screen for drugs for cancer therapy. Thus, this application also preferably represents a use in a screening procedure for drugs for treating colorectal carcinomas and/or metastases, preferably liver metastases.

As already discussed above, the gene sequences, oligonucleotides or other fragments of the marker genes of this invention can be used to detect and quantify the differential gene expression. Qualitative and quantitative methods for such measurements are known in molecular genetics. In addition, antibodies can be produced against an epitope or a protein or several epitopes or several proteins which were produced by the genes described in this invention. These antibodies can be used to quantify the protein content in a human cell. Protocols for detecting and quantifying the protein expression by monoclonal or polyclonal antibodies are well known in molecular genetics. Examples are ELISA, RIA and FACS analysis.

The gene sequences or gene fragments, or complementary nucleic acids of the marker genes of this invention can also be used in gene therapy. For example, the cDNA or one or more genes of the invention can be introduced ex vivo into target cells. After stable integration and transcription or translation have been ensured, the cells in the tumors of the patients can be returned and they potentially can correct mutations connected with the underexpression or overexpression of these proteins. Alternatively, the cDNA of one or more genes of the invention can be introduced in vivo by using vectors such as, for example, retroviruses, adenoviruses, HS viruses or bacterial plasmids. The inclusion of cationic liposomes, polylysine conjugates or the direct injection of DNA, in addition, represent nonviral methods.

An additional use of the invention is the possibility of therapeutically using the proteins coded by the marker genes of the invention, their ligands, or complementary nucleic acid sequences. In particular, the combination of different such drugs can have a synergistic effect on tumor reduction, or it can result in no metastasis occurring. Moreover, these drugs can also be used synergistically with substances that are already established in the therapy of colorectal carcinoma.

The selected genes which form the basis of the present invention are preferably used in the form of a “minimal set” or in the form of a “gene panel,” that is a collection which comprises the special genetic sequences of the present invention and/or their expression products. The formation of gene panels allows a rapid and specific analysis of specific aspects of cancer diseases, particularly carcinoma, particularly an adenocarcinoma, preferably a gastrointestinal carcinoma, and particularly preferably a colorectal carcinoma, metastasis formation of particularly colon tumors, and also the response to specific chemotherapeutic treatment procedures, particularly a 5-FU-containing chemotherapy/combination therapy. As described and used in this invention, the gene panels can be used with a surprisingly high efficiency for the diagnosis, treatment and monitoring, and also for the analysis of a predisposition for intestinal cell proliferative diseases. In particular, the marker genes represented here (or a subset, minimal set thereof) can be used in the form of gene panels to be analyzed (gene expression panels) in in vitro diagnostics. In addition, the use of a diverse array of the genes used here allows a relatively high level of sensitivity and specificity in comparison to individual analyses. Of the genes whose different gene (expression) profile is described here, the special combination of the genes according to the above-described invention, which is also characterized in the claims, makes available a particularly sensitive and specific means for identifying proliferative cell diseases of intestinal tissues, particularly the colon mucosa and metastases originating therefrom.

The gene panels described and used in this invention can be used with surprisingly high efficiency for treating and determining the treatment course of proliferative diseases of intestinal cells, (particularly of the colon), by predicting the outcome of the treatment with a therapy comprising one or more 5-FU-containing active ingredients. The analysis of each gene of the panel contributes to the evaluation of the patient responsiveness so that, in a less preferred embodiment, the patient evaluation can be achieved by analyzing only one gene. The analysis of a single member of the “gene panel” would make available an inexpensive but less precise means for evaluating the patient responsiveness; the analysis of several members of the panel would make available a decidedly more expensive means for carrying out the method of the invention which, however, would be more precise (the technically preferred solution). The same applies, mutatis mutandis, for the determination of a carcinoma, particularly an adenocarcinoma, preferably a gastrointestinal carcinoma, and particularly preferably a colorectal carcinoma, and the determination described herein of metastases, particularly liver metastases. Therefore, as a rule, the methods according to the invention comprise at least the investigation of the expression profile of two, preferably at least three, preferably at least four, preferably at least five, preferably at least six, preferably at least seven, preferably at least eight, preferably at least nine, preferably at least ten, preferably at least 15, preferably at least 20, preferably at least 25, preferably at least 30, preferably at least 35, preferably at least 40, preferably at least 45, preferably at least 50, preferably at least 55, preferably at least 60, preferably at least 65, preferably at least 70, preferably at least 75, preferably at least 80, preferably at least 85, preferably at least 90, preferably at least 95, preferably at least 100, and preferably at least 110 of the 120 marker genes described herein.

The methods according to the present invention are used for the improved detection, for the treatment and monitoring of proliferative diseases of intestinal cells, particularly of the colon mucosa.

The present invention, moreover, relates to the diagnosis and/or prognosis of events which can be detrimental or relevant to the patients or individuals, in which procedures the gene expression of the 120 marker genes represented here (or a minimal set thereof), can be compared with another set of gene expression parameters (for example, of healthy controls or of 5-FU-responders/nonresponders), where the differences are used as the basis for the diagnosis and/or prognosis of events which are detrimental or relevant to patients or individuals.

In connection with the present invention, the expression “chemotherapy” denotes the use of active ingredients of chemical substances, particularly 5-FU-containing substances and substance mixtures, for treating cancer and/or metastases.

The invention will be explained with greater precision on the basis of the following sequences, tables and examples, without being limited to them.

The tables show:

Table 1 contains the genes which are expressed differentially in the present invention between a primary tumor and liver metastases. (Further explanations for Table 1 can be found in the description of the invention.)

Table 2 contains the genes which are expressed differentially in the present invention between liver metastases of responders and non-responders to a 5-FU-containing therapy. (Further explanations for Table 2 can be found in the description of the invention.)

Table 3 contains the annotations, gene names and Affymetrix ID numbers of the genes described in Tables 1-3.

Table 4 expression data after the statistical evaluation according to Example 4b of responders (Resp) versus non-responders (Non-Resp) to a 5-FU-containing palliative chemotherapy.

Table 5 expression data of responders (Resp), non-responders (Nonresp) and patient T. of Example 16

Table 6 expression data of colon/rectum mucosa (healthy) versus colorectal (primary) tumor

Table A Prediction of the response of liver metastases to a 5-FU-containing chemotherapy by the comparison of responders (Resp; n=10) and non-responders.

Table B Early detection of a colorectal carcinoma by the comparison of healthy mucosa (Muc; n=3) and colorectal carcinoma

Table C Prediction of liver metastases by the comparison of liver metastases (Lm; n=20) and colorectal carcinoma

Table D Congruence list of the assigned SEQ ID Nos.

The figures show:

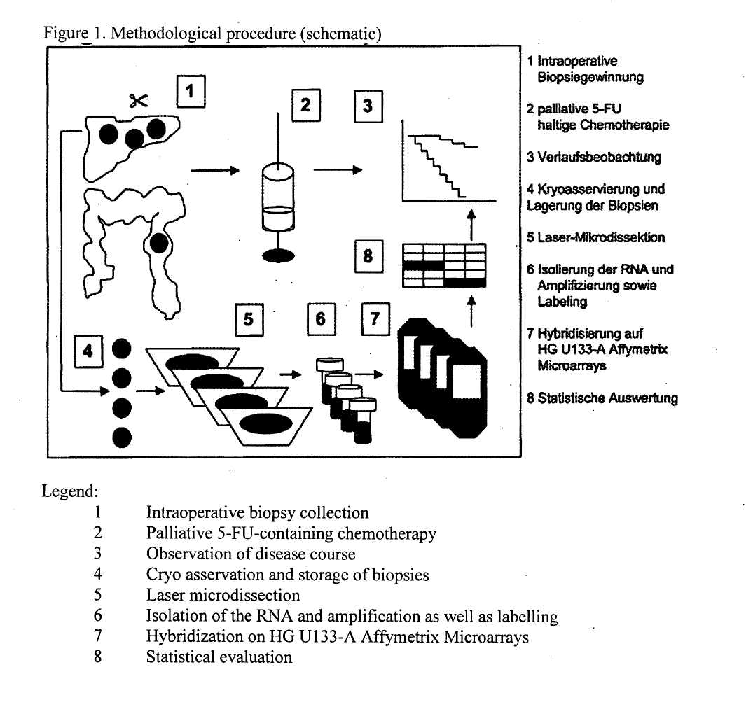

FIG. 1 shows schematically the methodological procedure which is used for the establishment of a gene expression profile with predictive significance, and thus are [sic] therefore necessary for the invention. (Further explanations can be found in the examples of the invention.)

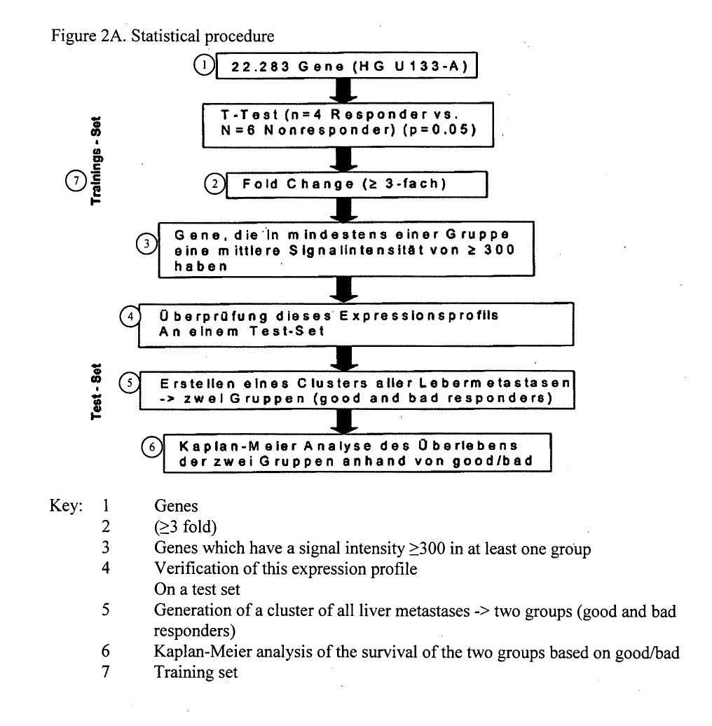

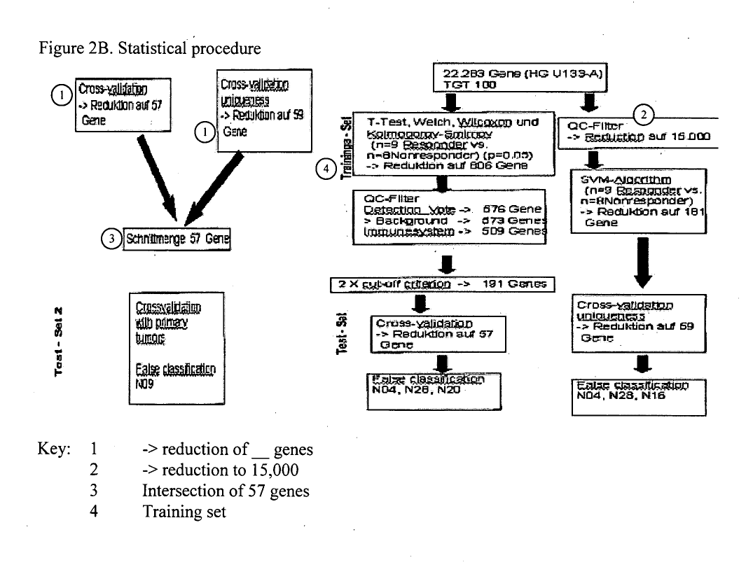

FIG. 2 shows the course of the statistical evaluation using a training test set method. Under A the procedure is shown as carried out in Example 4a, while under B the procedure used in Example 4b is shown. (Further explanations can be found in the examples of the invention.)

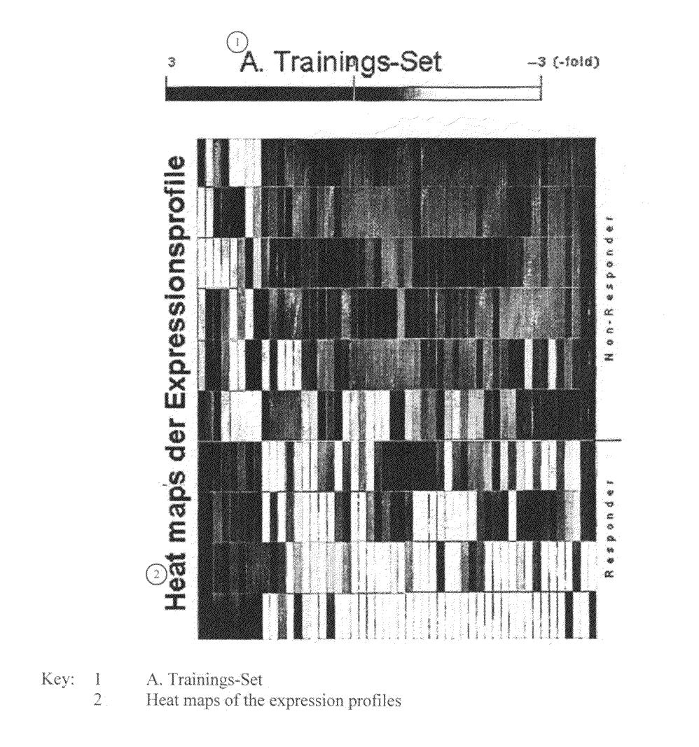

FIG. 3 shows “heat maps” of the expression profiles, which supplies via mathematical algorithms the similarity of the signatures and undertakes a subdivision into groups. FIG. 3A. shows the supervised cluster analysis of the training set, and FIG. 3b the cluster analysis carried out subsequently with a test set which is independent of the training set. (Further explanations can be found in the examples of the invention.)

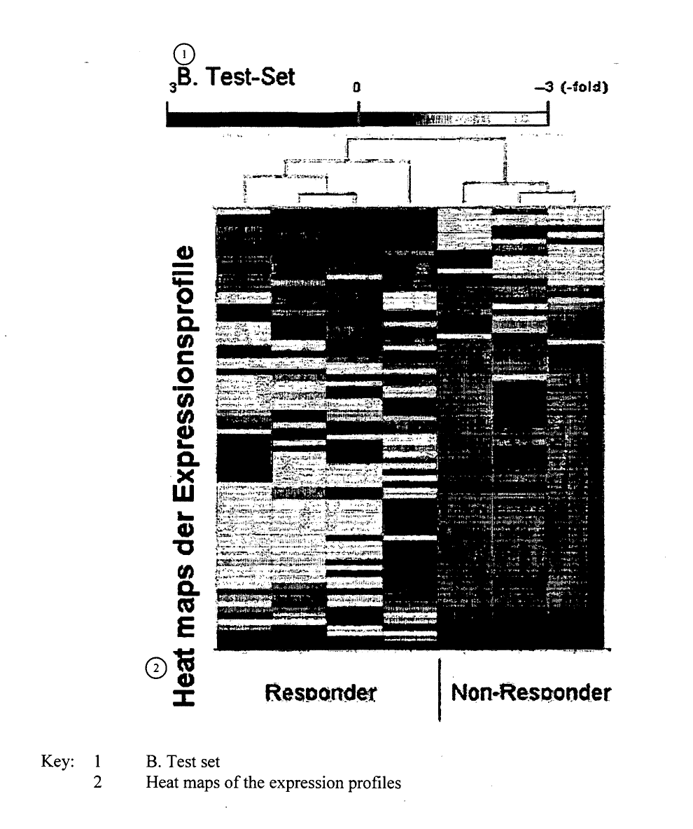

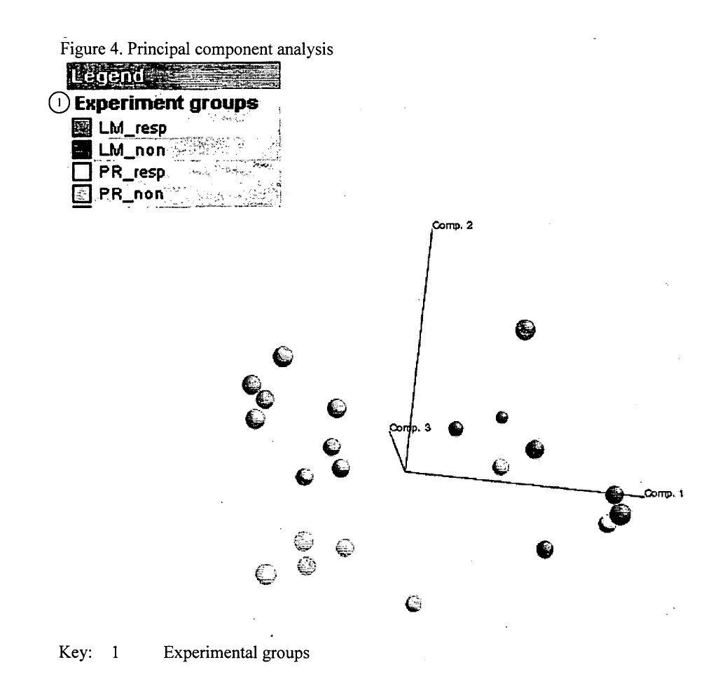

FIG. 4 shows the evaluation of the Principal Component Analysis. Each sphere symbolizes here the gene expression profile from a tissue (tumor or metastasis). The spatial proximity of the spheres of both the responders (green) and the non-responders (red brown) to the 5-FU-containing chemotherapy, and the spatial separation between green and red brown spheres shows [sic] the good possibility for discriminating between the two forms of the response. (Further explanations can be found in the examples of the invention.)

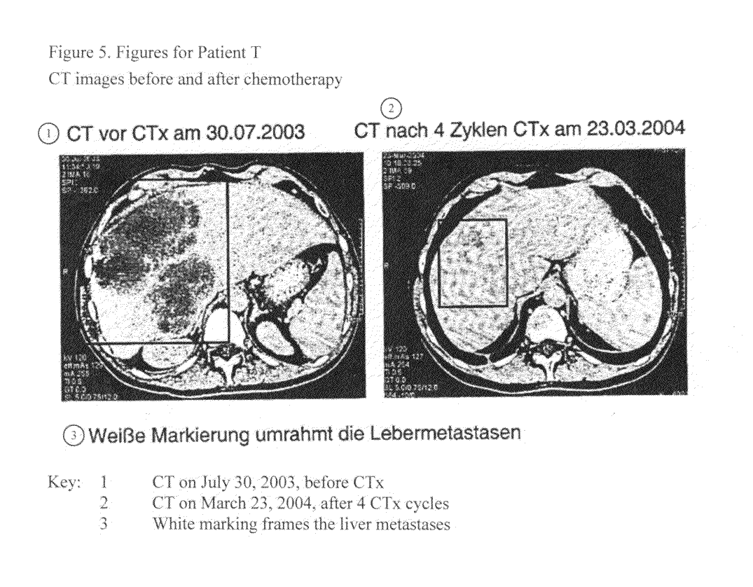

FIG. 5 shows computer tomography (CT) views of patient T. before and after the first-line treatment with a 5-FU-containing therapy. Here the regression in the size of the liver metastases (red bordered) indicates the start of the treatment. This is a partial remission (PR), that is a response to the chemotherapy in accordance with the WHO criteria. This patient also shows the response profile in the gene expression profile from the represented liver metastasis. (Further explanations can be found in the examples of the invention.)

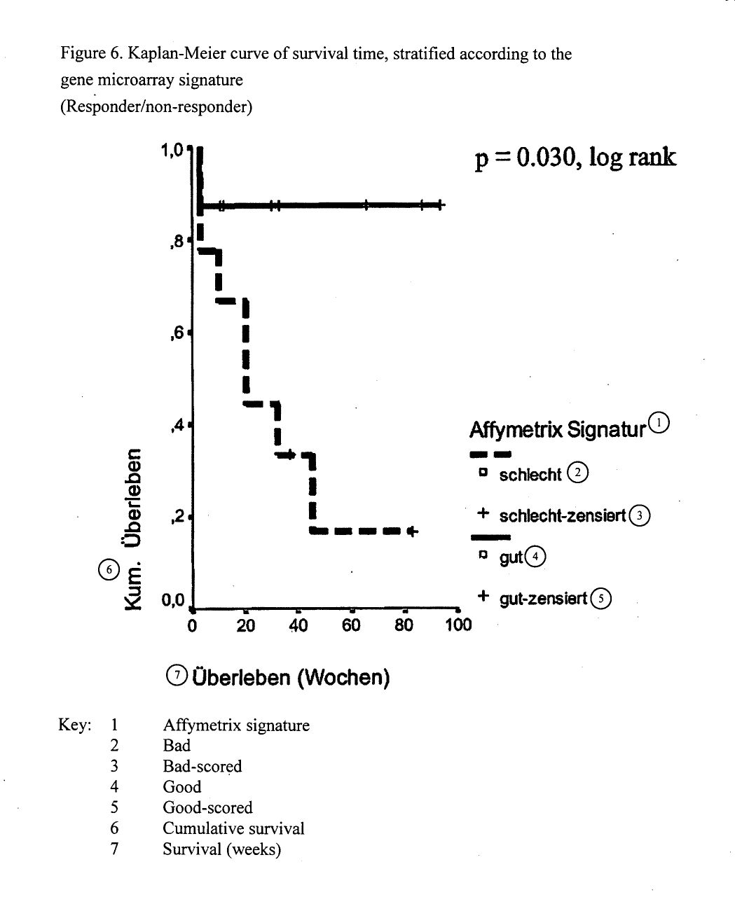

FIG. 6 shows a Kaplan-Meier (death) curve. The survival time in weeks is plotted on the x axis, and on the y axis the cumulative survival or death in %. The stratification was carried out in accordance with the gene expression profile between responders (solid line) and non-responders (broken line). The advantage in terms of patient survival can be seen in the response signature. The log rank analysis yielded a statistical significance of p=0.030. (Further explanations can be found in the examples of the invention.)

The following examples illustrate the invention without limiting it in any way.

### EXAMPLE 1

### Obtention and Purification of the Tissue

The tissue samples of normal colon mucosa, colorectal carcinoma and the corresponding liver metastases are obtained intraoperatively, frozen immediately in liquid nitrogen, and then archived at −80° C.

Using a cryotome, 5-μm thick sections of the tumor samples are prepared, applied to a glass slide, and stained with hematoxylin eosin (HE). Only tumor samples which contain a tumor cell portion, determined by microscopy, of at least 80% are chosen for further processing.

Then, using the cryotome, 30-μm thick sections of the selected tumor specimens are prepared, applied to special membrane slides (membrane slides for laser microdissection; Molecular Machines & Industries AG; CH-8152 Glattbrugg), and immediately transferred to 70% ethanol.

For the preparation of the laser microdissection, the following incubation steps were then carried out:

1. 30 sec in Mayer's hemalum solution

2. rinsing in RNase-free water

3. allowing color change to blue for approximately 30 sec in RNase-free water

4. washing for 30 sec in 70% ethanol

5. washing for 60 sec in 95% ethanol

6. 30 sec in eosin Y solution

7. washing for 60 sec in 95% ethanol

8. washing for 60 sec in 95% ethanol

9. washing for 60 sec in 100% ethanol

10. washing for 5 min in 100% xylene, and

11. drying of the sections in air and possibly removal of xylene residue with a lint-free cloth

The tumor cell areas were then cut out with a laser microdissection apparatus (LMD) from Leica, and transferred into a reaction vessel. Then the total RNA was isolated from the tumor cell areas according to the manufacturer's protocol using an RNeasy Mini-Kit (Order No. 74104) from Qiagen. Appropriate protocols can be obtained from the manufacturer, or are published on the Internet.

The quality of the isolated total RNA was verified with the help of a Lab-on-a-Chip apparatus (Agilent), and only quantitatively sufficient total RNA (>100 ng) as well as high-quality RNA are used for the following work steps.

### EXAMPLE 2

### Preparation and Amplification of a RNA from Isolated Total RNA

The preparation of amino-allyl labeled cRNA (aRNA) from the total RNA and its amplification was carried out according to the Eberwine method (van Geldern R N et al. (1990); PNAS USA 87:1663-7) and using the MessageAmp® kit from Ambion according to the manufacturer's protocol. Appropriate protocols can be obtained from the manufacturer, or are published on the Internet.

The labeled cRNA was purified and concentrated with the “RNeasy mini” kit from Qiagen. The work steps required for this purpose were carried out according to manufacturer's protocol, which is also published on the Internet.

### EXAMPLE 3

### Use of the Affymetrix Gene Arrays

The probe which is to be used for the hybridization was prepared by fragmenting labeled cRNA. Then, HG U133-A microarrys [sic; microarrays] from the company Affymetrix were hybridized with this probe, unbound probe was removed, and the raw data were read with the GeneArray Scanner Agilent. The work steps for carrying out the mentioned procedures were carried out in accordance with the instructions in the Handbook for Gene Expression Analysis (Affymetrix). The signal intensities and the detection signals (“detection calls”) were established with the help of the GeneChip 5.0 Software from Affymetrix.

### EXAMPLE 4

### Statistical Evaluation of the Data Obtained

The data collected with the GeneArray Scanner on the hybridization of the labeled cRNA fragments with Microarray sequences were then evaluated statistically. The evaluation was carried out in two ways:

a) The method comprised the establishment of a training set and the verification of the data obtained on a test set.

1. The data sets were “published,” that is made comparable, using Affymetrix® Software.

2. Preliminary evaluation using the Affymetrix Data mining tool (average Resp, average Non-Responder, fold-change, t-test Resp. vs. Non-responder p<0.05). The training set (n=10) was compiled randomly. 726 genes are expressed significantly differently.

3. The data are transferred to Excel. Then, the genes that were expressed with a significant difference in the t-test are reduced further by reduction to genes which are expressed with >3 fold difference. Reduction to 168 genes.

4. The additional filtration is carried out by reduction to genes which present an average signal value in at least one group of >300. Reduction to 82 genes.

5. Removal of genes that are listed twice or multiple times on the chip. Reduction to 72 genes.

6. The expression profile of the 72 genes (also represented in Table 2) is used with the test set (n=7) and clustered hierarchically based on correlation (Spotfire® Software). Two clusters are produced which correspond 100% to the responders and non-responders. The survival times of the patients (time of death or last date of life minus time of operation) are calculated, and a Kaplan-Meier curve with the variable “bad”-“good” response is established. In the log-rank test, the significance determined for the total survival is p=0.04.

b) An alternative method for the statistical evaluation of the raw data started with the available 30 data sets and formed five groups therefrom: Group_{—}1 LM responder, Group_{—}2 LM non-responder; Group_{—}3 PR responder; Group_{—}4 PR non-responder and Group_{—}5 with undefined clinical response. The finding phase was subdivided into two parallel sections A and B. In the finding section A, using the t-test, Welch, Wilcoxon and Kolgomorov-Smirnov analysis, the statistically significant differentially regulated genes were identified distinguishing between the Groups_{—}1 and _{—}2 using data sets with n=8 and n=9, respectively. In the process, the cutoff level for the significance was set at p=0.05. The analysis showed that one group of 806 genes of the entire gene pool of 22,283 genes was significant. In a second step, these genes were subjected to a quality analysis, which showed that 230 genes could not be validly measured using the Affymetrix gene chip system, and an additional 3 genes were below the set detection threshold of 30 RLU [relative light units]. In an additional analysis step, all those genes were separated from the remaining 573 genes which, with respect to their biological function, must be considered to be connected with the inflammatory reactions or the immune system. This was done on the basis of gene lists established beforehand, and on literature data—such as the data banks (NCBI, OMIM [Online Mendelian Inheritance in Men], UNIGENE). In order to identify as having predictive value only those genes whose relative expression change is clearly measurable, another process was carried out to remove each gene whose delta in the expression between Group_{—}1 and Group_{—}2 is greater than a factor 2. This criterion was satisfied by 191 genes. In a crossvalidation step, kNN (k nearest neighbor) analysis and the inclusion of Group_{—}5, as well as the use of a so-called gene ranking, allowed the reduction of the genes to 55. Using these genes in the kNN analysis, only three data sets were classified as incorrect with k=3 and 25% test set fraction, as well as 10,000 iterations.

In the second independent finding attempt [unconfirmed translation], starting from the same Groups_{—}1 and _{—}2, and following a previous quality analysis of 22,283 genes, only 15,000 were considered to be valid (expressed with sufficient differentiation) in this analysis construction. Using these approximately 15,000 genes, a gene reduction was carried out based on SVM (support vector machines) algorithms, which were shown to be particularly well suited to solve n-dimensional problems with two or more classes (Weston and Watkins, Proceedings of the Seventh European Symposium On Artificial Neural Networks, April 1999; Vapnik, The Nature of Statistical Learning Theory, 1995, Springer, N.Y.; Vapnik, Statistical Learning Theory, 1998, Wiley, New York; Burges, Data Mining and Knowledge Discovery 2(2):955-974, 1998). Using these algorithms, a hyperplane (separation plane) was defined in the n-dimensional space; it consisted of the “vectors” of the individual genes and their expression level. The reduction yielded 181 genes required for a predictive separation. After checking the data for “duplicate” listings which result from the multiple representation of genes on the GeneChip Microarray, and the reduction of immune system genes, 57 genes were available for a first crossvalidation, as already described previously. The cut-off quantity of the two gene groups from the analysis sections A and B was 55 (55 genes, as listed in Table 3). Now, in the next analysis section C, the so far unused data sets of Groups_{—}3 and _{—}4 were used in a validation with kNN algorithms and in a PCA (principle component analysis). With the exception of one data set from Group_{—}4, all the primary tumor samples were assigned correctly to the appropriate “responder” or “non responder” groups.

Thus, the invention makes available 72 and 55 (marker) genes whose expression profile and/or expression level can be used both for (early) detection of a colorectal carcinoma, for the prediction of metastases (preferably liver metastases) dependent on a primary colorectal carcinoma and/or for the prediction of the response of metastases to a 5-FU-containing therapy (chemotherapy). Because the genes presented here, of 72 and 55 genes, have overlaps, a total of 120 genes is made available here (represented in SEQ ID NO: 1 to SEQ ID NO: 181, and shown in Tables 2 and 3, and also in Table D), with which the person skilled in the art can carry out the method according to the invention, as well as the use of the invention. SEQ ID NO: 1-120 here relate to individual genes which corresponds to the 120 marker genes. The additional sequences, as represented in SEQ ID NO: 121-181, are variants of individual marker genes. The corresponding correlation of the individual marker genes (120) with the sequences (181) can be obtained from the tables, particularly Table D. The “kits” of the invention are defined by, and newly prepared using, these 120 genes.

The tables in the appendix (particularly Tables 1-5 and A, B, C, and D) allow the person skilled in the art to determine and to analyze the selected expression profiles.

### EXAMPLE 5

### 72 Genes and a Selection Thereof from Table 2 which are Suitable as Marker Genes for the Detection of a Colorectal Carcinoma

The values of column 7 in Table 2 show a significant differential expression of the 72 genes described for colon mucosas and colorectal carcinomas. From them, one can disclose the use of these genes as marker genes for the detection of a colorectal carcinoma. Based on a particularly high significance of p<0.05 in the t-test (column 7, Table 2), the following gene selection (18 genes) is particularly well suited as marker genes for the detection of a colorectal carcinoma: major histocompatibility complex, class II, DP beta [Affymetrix number 201137_s_at], SEQ ID No. 2; CD163 antigen [Affymetrix number 203645_s_at], SEQ ID number 7; phospholipase C, beta 4 [Affymetrix number 203895_at], SEQ ID number 8 or 122; solute carrier family 31 (copper transporters), member 2 [Affymetrix number 204204_at], SEQ ID number 12; group-specific component (vitamin D binding protein) [Affymetrix number 204965_at], SEQ ID number 19; profilin 2 [Affymetrix number 204992_s_at], SEQ ID number 21 or 124; LGN [lateral geniculate nucleus] protein [Affymetrix number 205240_at], SEQ ID number 25; arylacetamide deacetylase (esterase) [Affymetrix number 205969_at], SEQ ID number 33; Down syndrome critical region gene 6 [Affymetrix number 207267_s_at], SEQ ID number 38; peroxisome proliferative activated receptor, gamma [Affymetrix number 208510_at], SEQ ID number 41, 136, 137 or 138; allograft inflammatory factor 1 [Affymetrix number 209901_x_at], SEQ ID number 45, 139 or 140; major histocompatibility complex, class II, DP alpha 1 [Affymetrix number 211991_s_at], SEQ ID number 49; aldehyde dehydrogenase 1 family member A1 [Affymetrix number212224 at], SEQ ID number 50; plexin D1 [Affymetrix number212235_at], SEQ ID number 51; hypothetical protein PP1665 [Affymetrix number 213343_s_at], SEQ ID number 54; complement component 3 [Affymetrix number 217767_at], SEQ ID number 66; glucosaminyl (N-acetyl) transferase 3, mucin type [Affymetrix number 219508_at], SEQ ID number 68; A disintegrin-like and metalloprotease with thrombospondin type 1 [Affymetrix number 222162_s_at], SEQ ID number 70.

In diagnostic use, the value of one or more (preferably at least two) of the described genes or their translated proteins are determined, for example, in blood. The determination of RNA in blood is carried out by isolating RNA from citrated plasma, transcription to cDNA, and subsequent PCR. The determination of proteins in blood can be carried out by Western blot, ELISA or Luminex® technology. The values so obtained are compared with the normal value of an individual (or with healthy tissue from the patient) to be able to obtain an early diagnosis of a colorectal carcinoma.

For detecting a colorectal carcinoma or for early diagnosis, the person skilled in the art investigates preferably at least one, more preferably at least two, and most preferably at least 3 of the marker genes represented here, in the form of their expression profile. Most advantageously, the expression profile is defined by the following genes: LGN protein [Affymetrix number 205240_at], SEQ ID number 25; peroxisome proliferative activated receptor, gamma [Affymetrix number 208510_s_at], SEQ ID number 41, 136, 137 or 138; and glucosaminyl (N-acetyl) transferase 3, mucin type [Affymetrix number 219508_at], SEQ ID number 68.

### EXAMPLE 6

### 55 Genes and a Selection Thereof from Table 3, which are Suitable as Marker Genes for the Detection of a Colorectal Carcinoma

The values of column 7 in Table 3 (t-test) show a significant differential expression of the 55 described genes for colon mucosas and colorectal carcinomas. From this one discloses the use of these genes as marker genes for the detection of a colorectal carcinoma. Based on a particularly high significance of p<0.05 in the t-test (column 7, Table 3), the following gene selection (18 genes) is particularly well suited for use as marker genes for the detection of a colorectal carcinoma: LGN protein [Affymetrix number 205240_at], SEQ ID number 25; peroxisome proliferative activated receptor, gamma [Affymetrix number 208510_{—}5_at], SEQ ID number 41, 136, 137 or 138; glucosaminyl (N-acetyl) transferase 3, mucin type [Affymetrix number 219508_at], SEQ ID number 68; phosphodiesterase 4B, cAMP-specific (phosphodiesterase E4 dunce homolog, Drosophila) [Affymetrix number 203708_at], SEQ ID number 78 or 157; solute carrier family 26 (sulfate transporter), member 2 [Affymetrix number 205097_at], SEQ ID number 83; pleiomorphic adenoma gene 1 [Affymetrix number 205372_at], SEQ ID number 85; KIAA0672 gene product [Affymetrix number 205414_s_at], SEQ ID number 86; PTK7 protein tyrosine kinase 7 [Affymetrix number 207011_s_at], SEQ ID number 92, 159, 160, 161 of 162; homeobox A9 [Affymetrix number209905_at], SEQ ID number 99 or 170; interleukin 1 receptor, type II [Affymetrix number 211372_s_at], SEQ ID number 101, 171 or 172; protein O-fucosyltransferase 1 [Affymetrix number 212349_at], SEQ ID number 103 or 174; lipocalin 2 (oncogene24p3) [Affymetrix number 212531_at], SEQ ID number 104; paraneoplastic antigen [Affymetrix number 213230_at], SEQ ID number 105; immunoglobulin lambda joining 3 [Affymetrix number 214677_x_at], SEQ ID number 109 or 177; chromosome 14 open reading frame 18 [Affymetrix number 217988_at], SEQ ID number 112, 178, 179 or 180; heparan sulfate (glucosamine) 3-O-sulfotransferase 2 [Affymetrix number 219697_at], SEQ ID number 114; LBP [lipopolysaccharide binding protein] protein; likely ortholog of mouse CRTR-1 [Affymetrix number 219735_s_at], SEQ ID number 115; and aryl hydrocarbon receptor nuclear translocator-like 2 [Affymetrix number 220658_s_at], SEQ ID number 118.

In diagnostic use, the values of one or more (preferably at least two) of the described genes or their translated proteins are determined, for example in blood, and compared with the normal values of an individual, to allow early diagnosis of a tumor disease. The “normal values of an individual” can be the values of an individual who is not sick, or as mentioned above, of healthy tissue from the sick patient.

### EXAMPLE 7

### Selection of Genes from Tables 2 and 3 which are Particularly Well Suited as Marker Genes for the Detection of a Colorectal Carcinoma

The precise selection from the 72 genes of Table 2 (Example 5) combined with the gene selection from the 55 genes of Table 3 (Example 6), represents a first “minimal set” of 33 marker genes, which, in reference to a particularly significantly different expression, measured with p<0.05 in the t-test (Table 2 and Table 3, column 7), stands out from the quantity of the total 120 marker genes and is therefore of special diagnostic relevance. The genes are: major histocompatibility complex, class II, DP beta 1 [Affymetrix number 201137_s_at], SEQ ID number 2; CD163 antigen [Affymetrix number 203645_s_at], SEQ ID number 7; phospholipase C, beta 4 [Affymetrix number 203895_at], SEQ ID number 8 or 122; solute carrier family 31 (copper transporters), member 2 [Affymetrix number 204204_at], SEQ ID number 12; group-specific component (vitamin D binding protein) [Affymetrix number 204965_at], SEQ ID number 19; profilin 2 [Affymetrix number 204992_s_at], SEQ ID number 21 or 124; LGN protein [Affymetrix number 205240_at], SEQ ID number 25; arylacetamide deacetylase (esterase) [Affymetrix number 205969_at], SEQ ID number 33; Down syndrome critical region gene 6 [Affymetrix number 207267_s_at], SEQ ID number 38; peroxisome proliferative activated receptor, gamma [Affymetrix number 208510_s_at], SEQ ID number 41, 136, 137 or 138; allograft inflammatory factor 1 [Affymetrix number 209901_x_at], SEQ ID number 45, 139 or 140; major histocompatibility complex, class II, DP alpha 1 [Affymetrix number 211991_s_at], SEQ ID number 49; aldehyde dehydrogenase 1 family, member A1 [Affymetrix number 212224_at], SEQ ID number 50; plexin D1 [Affymetrix number 212235_at], SEQ ID number 51; hypothetical protein PP1665 [Affymetrix number 213343_s_at], SEQ ID number 54; complement component 3 [Affymetrix number 217767_at], SEQ ID number 66; glucosaminyl (N-acetyl) transferase 3, mucin type [Affymetrix number 219508_at], SEQE ID number 68; A disintegrin-like and metalloprotease with thrombospondin type 1 [Affymetrix number 222162_s_at], SEQ ID number 70; phosphodiesterase 4B, cAMP-specific (phosphodiesterase E4 dunce homolog, Drosophila) [Affymetrix number 203708_at], SEQ ID number 78 or 157; solute carrier family 26 (sulfate transporter), member 2 [Affymetrix number 205097_at], SEQ ID number 83; pleiomorphic adenoma gene 1 [Affymetrix number 205372_at], SEQ ID number 85; KIAA0672 [expansion unknown/perhaps, Kasuza Integrative Autoannotation] gene product [Affymetrix number 205414_s_at], SEQ ID number 86; PTK7 protein tyrosine kinase 7 [Affymetrix number 207011_s_at], SEQ ID number 92, 159, 160, 161 or 162; homeobox A9 [Affymetrix number 209905_at], SEQ ID number 99 or 170; interleukin 1 receptor, type II [Affymetrix number 211372_s_at], SEQ ID number 101, 171 or 172; protein O-fucosyltransferase 1 [Affymetrix number 212349_at], SEQ ID number 103 or 174; lipocalin 2 (oncogene 24p3) [Affymetrix number 212531 at], SEQ ID number 104; paraneoplastic antigen [Affymetrix number 213230_at], SEQ ID number 105; immunoglobulin lambda joining 3 [Affymetrix number 214677_x_at], SEQ ID number 109 or 177; chromosome 14 open reading frame 18 [Affymetrix number 217988_at], SEQ ID number 112; heparan sulfate (glucosamine) 3-O-sulfotransferase 2 [Affymetrix number 219697_at], SEQ ID number 114; LBP protein; likely ortholog of mouse CRTR CP-2-related transcriptional response-1 [Affymetrix number 219735_s_at], SEQ ID number 115; and aryl hydrocarbon receptor nuclear translocator-like 2 [Affymetrix number 220658_s_at], SEQ ID number 118. Preferably, one again determines 18 genes from the gene set presented here with 33 genes, where the former 18 genes can be used for the early detection of a colorectal carcinoma. A preferred selection of these 18 marker genes comprises, for example, the genes with SEQ ID NOs: 2, 7, 8 (or 122), 12, 19, 21 (or 124), 25, 33, 38, 41 (or 136, 137, 138), 45 (or 139, 140), 49, 50, 51, 54, 66, 68 and 70 (see also Example 5) or the genes with the SEQ ID NOs: 25, 41 (or 136, 137, 138), 68, 78 (or 157), 83, 85, 86, 92 (or 159, 160, 161, 162), 99 (or 170), 101 (or 171, 172), 103 (or 174), 104, 105, 109 (or 177), 112, 114, 115 and 118 (see also Example 6). The person skilled in the art certainly can use the results represented here for making other diagnostic selections. The possibilities available to him include optionally to determine from the tables in the appendix what the relevant diagnostic marker genes are and to make the appropriate selection. The precise marker selections that are mentioned herein as preferred comprise preferred embodiments. Thus, the person skilled in the art can certainly combine the 18 genes as represented in Example 6.

By comparing the genes which, according to Table 2 and Table 3, present differential expression between colorectal carcinomas and mucosa, three genes were identified which were taken into consideration in both selections (Example 5 and Example 6), and which present a particularly significant differential expression. These three genes should be considered a preferred embodiment of this invention for the early detection of a colorectal carcinoma. It is preferred to determine at least one of these three genes, more preferably at least two and most preferably all three genes. The person skilled in the art will also make use of the expression of additional genes (for example, chosen from the “minimal sets” of the 72, 55, 33, and 18 genes mentioned here) for the diagnosis. The special selection of these three genes represents an additional “minimal set,” which is of great diagnostic relevance. The genes are: LGN protein [Affymetrix number 205240_at], SEQ ID number 25; peroxisome proliferative activated receptor, gamma [Affymetrix number 208510_s_at], SEQ ID number 41, 136, 137 or 138; glucosaminyl (N-acetyl) transferase 3, and mucin type [Affymetrix number 219508_at], SEQ ID number 68.

The genes of the “minimal sets” represented here are of particular interest in the framework of a preventive care cancer investigation, because these 33, 18, 18 or three genes are particularly well suited as marker genes for detecting a colorectal carcinoma. Corresponding data can also be obtained from Table B.

Table B shows the difference in the expression profile/expression level of the defined genes in comparison to the healthy mucosa (Muc) and the tumor tissue (Tu). A “↓” here means that the corresponding gene in mucosa compared to tumors has a low expression level, while “↑” means that the corresponding gene has a higher expression in healthy tissue compared to the tumor tissue. Reversing the conclusion, this means that “↓” in Table B means that the corresponding gene exhibits a higher expression level in the tumor tissue than in the healthy mucosa, and “↑” means that the corresponding gene presents a lower expression level in the tumor tissue than in the healthy mucosa.

In Example 8 and in the following examples it is shown that the 120 genes (SEQ ID NOs: 1-181) according to the invention are also suitable for detecting a liver metastasis of a colorectal carcinoma. Thus, a liver metastasis dependent upon a colorectal carcinoma, preferably an already diagnosed colorectal carcinoma, can be determined.

### EXAMPLE 8

### The 72 Genes from Table 2 can Also be Used for the Prediction of Liver Metastases of a Colorectal Carcinoma

The values from column 10 of Table 2 show a differential expression of the 72 genes described, allowing differentiation between a colorectal carcinoma and corresponding liver metastases. In column 2, the t-test between liver metastases compared to the tumor is represented. Therefore, these genes are suitable as marker genes for the prediction of liver metastases in the case of a diagnosed colorectal carcinoma.

### EXAMPLE 9

### Minimal Selection (“Minimal Set”) of Genes from Table 2, which are Particularly Well Suited for the Prediction of Liver Metastases in the Case of a Colorectal Carcinoma

Taking into consideration the significance starting at a value below 0.05 in the t-test in column 10 of Table 2, 20 genes were identified which presented a particularly clear differential expression between a colorectal carcinoma and corresponding liver metastases (see also Table C, first column). The genes are: aldolase B, fructose-bisphosphate [Affymetrix number 204705_x_at], SEQ ID number 17; group-specific component (vitamin D binding protein) [Affymetrix number 204965_at], SEQ ID number 19; fibrinogen, B beta polypeptide [Affymetrix number 204988_at], SEQ ID number 20; orosomucoid 1 [Affymetrix number 205041_s_at], SEQ ID number 22; alpha-1-microglobulin/bikunin precursor [Affymetrix number 205477_s_at], SEQ ID number 26; fibrinogen A alpha polypeptide [Affymetrix number 205650_s_at], SEQ ID number 29 or 125; coagulation factor II (thrombin) [Affymetrix number 205754_at], SEQ ID number 30; pre-alpha (globulin) inhibitor, H3 polypeptide [Affymetrix number 205755_at], SEQ ID number 31; arylacetamide deacetylase (esterase) [Affymetrix number 205969_at], SEQ ID number 33; asialoglycoprotein receptor 2 [Affymetrix number 206130_s_at], SEQ ID number 35, 133; 134 or 135; amyloid P component, serum [Affymetrix number 206350_at], SEQ ID number 37; haptoglobin [Affymetrix number 208470_s_at], SEQ ID number 40; alpha-1 antitrypsin [Affymetrix number 211429_s_at], SEQ ID number 48, 151 or 152; transferrin [Affymetrix number 214063_s_at], SEQ ID number 58; complement component 4A [Affymetrix number 214428_x_at], SEQ ID number 59; similar to human Ig rearranged gamma chain mRNA, V-J-C region [Affymetrix number 214669_x_at], SEQ ID number 64; complement component 3 [Affymetrix number 217767_at], SEQ ID number 66; fibrinogen, gamma polypeptide [Affymetrix number 219612_s_at], SEQ ID number 69 or 156; C-reactive protein, pentraxin-related [Affymetrix number 37020_at], SEQ ID number 71; and hemopexin [Affymetrix number 39763_at] SEQ ID number 72.

The above-mentioned 20 genes represent a particularly suitable selection which, in the framework of a standard investigation of these marker genes, allows investigation for the presence or growth of liver metastases due to a primary colorectal carcinoma. In the corresponding expression profile/expression level of an individual gene to be determined in Table C, the “↑” means that the corresponding gene is upregulated in the metastases compared to the primary tumor, while “↓” means that the corresponding gene is down-regulated.

### EXAMPLE 10

### The 55 Genes from Table 3 can be Used for the Prediction of Liver Metastases in the Case of a Diagnosed Colorectal Carcinoma

The values from column 10 of Table 3 show a differential expression of the 55 genes described, allowing the differentiation between a colorectal carcinoma and corresponding liver metastases. Therefore, these genes are suitable as marker genes for the prediction of liver metastases in the case of an already diagnosed colorectal carcinoma; see Table 3.

### EXAMPLE 11

### Minimum Selection of Genes from Table 3 which are Particularly Well Suited for the Prediction of Liver Metastases in the Case of a Diagnosed Colorectal Carcinoma

Taking into consideration the significance starting at a value below 0.05 in the t-test in column 10 of Table 3, two genes were identified which produce a particularly clear differential expression between a colorectal carcinoma and corresponding liver metastases. The genes are: tenascin C (hexabrachion) [Affymetrix number 201645_at], SEQ ID number 74; immunoglobulin lambda joining 3 [Affymetrix number 214677_x_at], SEQ ID number 109 or 177; see also Table C. In the corresponding expression profile/expression level of an individual gene to be determined in Table C, “↑” means that the corresponding gene in the metastasis is upregulated compared to the primary tumor, while “↓” means that the gene is correspondingly down-regulated.

The above-mentioned two genes represent a particularly suitable selection which, in the framework of a standard investigation of these marker genes, allows the investigation of the growth of liver metastases due to a primary colorectal carcinoma.

It is preferred for these two genes to be optionally combined with other genes for corresponding diagnostics. In preferred diagnostics, these two genes are combined with at least one additional gene from Table C, Table 2 and/or Table 3; however, other genes of the SEQ ID NOs 1-73, 75-108, 110-176 and 178-181 are used for diagnosis. The corresponding information can also be found in column 10 of Tables 2 and 3.

### EXAMPLE 12

### Selection of genes of Tables 2 and 3 which are Particularly Well Suited for the Prediction of Liver Metastases in the Case of a Diagnosed Colorectal Carcinoma

The total of the 20 genes selected from Table 2 and the two genes selected from Table 3 represent the preferred “minimal set” of marker genes, and are therefore of very great diagnostic significance; see also Table C with the corresponding explanations in the preceding examples. The genes are: aldolase B, fructose-bisphosphate [Affymetrix number 204705_x_at], SEQ ID number 17; group-specific component (vitamin D binding protein) [Affymetrix number 204965_at], SEQ ID number 19; fibrinogen, B beta polypeptide [Affymetrix number 204988_at], SEQ ID number 20; orosomucoid 1 [Affymetrix number 205041_s_at], SEQ ID number 22; alpha-1-microglobulinlbikunin precursor [Affymetrix number 205477_s_at], SEQ ID number 26; fibrinogen A alpha polypeptide [Affymetrix number 205650_at], SEQ ID number 29 or 125; coagulation factor II (thrombin) [Affymetrix number 205754_at], SEQ ID number 30; pre-alpha (globulin) inhibitor, H3 polypeptide [Affymetrix number 205755_at], SEQ ID number 31; arylacetamide deacetylase (esterase) [Affymetrix number 205969_at], SEQ ID number 33; asialoglycoprotein receptor 2 [Affymetrix number 206130_s_at], SEQ ID number 35, 133, 134 or 135; amyloid P component, serum [Affymetrix number 206350_at], SEQ ID number 37; haptoglobin [Affymetrix number 208470_s_at], SEQ ID number 40; alpha-1 antitrypsin [Affymetrix number 211429_s_at], SEQ ID number 48, 151 or 152; transferrin [Affymetrix number 214063_s_at], SEQ ID number 58; complement component 4A [Affymetrix number 214428_x_at], SEQ ID number 59; similar to human Ig rearranged gamma chain mRNA, V-J-C region [Affymetrix number 214669_x_at], SEQ ID number 64; complement component 3 [Affymetrix number 217767_at], SEQ ID number 66; fibrinogen, gamma polypeptide [Affymetrix number 219612_s_at], SEQ ID number 69 or 156; C-reactive protein, pentraxin-related [Affymetrix number 37020_at], SEQ ID number 71; hemopexin [Affymetrix number 39763_at] SEQ ID number 72; tenascin C (hexabrachion) [Affymetrix number 201645_at], SEQ ID number 74; and immunoglobulin lambda joining 3 [Affymetrix number 214677_x_at], SEQ ID number 109 or 177.

The investigation of the expression level of these selected genes is of particularly great interest in the case of a diagnosed colorectal carcinoma, because the 22 genes can be used particularly well as marker genes for the detection of already pronounced, corresponding liver metastases.

The following examples demonstrate that the 120 genes identified here can also be used for the determination/prediction of the therapy course of a 5-FU-containing chemotherapy in the treatment of metastases, particularly of liver metastases.

Here, in the following examples, the investigation of liver metastases is emphasized.

### EXAMPLE 13

### 72 Genes from Table 1 which are Suitable for the Prediction of the Therapy Course of Patients with Liver Metastases of a Colorectal Carcinoma, in the Case of a 5-FU-Containing Chemotherapy or Combination Therapy

Comparing the gene expression in liver metastases of a colorectal carcinoma between patients who respond to a 5-FU-containing chemotherapy (“responder”) and patients who do not respond to a 5-FU-containing chemotherapy (“non-responder”), a significant differential expression was identified with 72 genes, see also Table 1 and Table A, first column.

One or more of the described genes, gene fragments, complementary nucleic acids or translated proteins are therefore well suited for predicting the efficacy of a 5-FU-based chemotherapy or combination therapy (5-FU, for example, in combination with folic acid, irinotecan, oxaliplatin and others).

In Table A, it is shown for this example (and the following examples) that the expression level of the individual genes can be determined and that a distinction can be made, particularly between “responders” and “non-responders.” Data for the 120 genes according to the invention are listed in Tables 1 and 4.

Table A is a representation of the genes that should be analyzed preferably. Here “t” means that the corresponding gene of patients who respond to a 5-FU-containing chemotherapy and/or combination therapy (“responders”) is expressed more strongly (“upregulated”) in comparison to patients who do not respond (“non-responders”). In contrast, the “↓” denotes a weaker expression of the corresponding gene in “responders” versus “non-responders.”

In general, in the context of this invention, the term expression level thus means a measurable value to be determined for the expression of the gene to be investigated, where this value, as shown in the tables and in Example 2, relates to the expression profile of the RNA in the sample. However, the methods represented here (for detecting a colorectal carcinoma, for predicting metastases, and also for predicting the response to a 5-FU therapy) can determine the expression level by other analyses (for example, determination of the protein quantity, the proteins, peptides or peptide segments coded by the genes SEQ ID NOs: 1-181). These analyses comprise, for example, and in a nonlimiting list, immunodetection methods such as Western blot, ELISA, RIA, immunoprecipitation, and also FACS analyses.

### EXAMPLE 14

### 55 Genes from Table 4 which are Suitable for the Prediction of the Therapy Course of Patients with Liver Metastases of a Colorectal Carcinoma, in the Case of a 5-FU-Containing Chemotherapy or Combination Therapy

By comparing the gene expression in liver metastases of a colorectal carcinoma between patients who respond to a 5-FU-containing chemotherapy and patients who do not respond to a 5-FU-containing chemotherapy, a significant differential expression was identified with 55 genes; see Table 4 and Table A (second column).

One or more of the described genes, gene fragments, complementary nucleic acids or translated proteins therefore are well suited for predicting the efficacy of a 5-FU-based chemotherapy or combination therapy (5-FU, for example, in combination with folic acid, irinotecan, oxaliplatin and others).

### EXAMPLE 15

### Selection of Genes from Tables 1 and 4 which can be Used as Marker Genes for Predicting the Therapy Course of Patients with Liver Metastases of a Colorectal Carcinoma in the Case of a 5-FU-Containing Chemotherapy or Combination Therapy

By comparing the gene expression in liver metastases of a colorectal carcinoma between patients who respond to a 5-FU-containing chemotherapy and patients who do not correspond to a 5-FU-containing chemotherapy, taking into consideration the significance levels in the comparative statistical analysis (t-, Welsh and Wilcoxon test); see Table A, columns 7, 8, 9 and 10, a total of 34 particularly significantly differentially expressed genes were identified in Tables 1 and 4. In addition to the statistical selection for reducing the gene list, the data from the “principal component analysis” (PCA) were also used. “Principal component analysis” (PCA) is a method which allows the classification of variables, based on correlative relationships, into mutually independent groups, and a reduction of the given existing dimensions to a few basic components. The result of the factor analysis yields mutually independent factors which explain the connections between the variables that are combined in them. Thus, it is a data reduction and hypothesis generating method which is suitable for verifying the dimensionality of complex characteristics. Using the “charges” (similar genes) of the individual constructs on the main components, the contents of the dimensions can be defined. In the framework of the factor analysis method, charges provide general information regarding how good a variable is with respect to a variable group. Here, those genes were not taken into consideration for the selection of the 28 genes in Table A, column 4, which in the first selection, see Table A, columns 1 and 2, presented a clear separation between liver metastases and primary tumor, independently of their predictive potential or response. Thus, the genes represented here represent the optimum with respect to response prediction and statistical significance.

These genes are: apolipoprotein E [Affymetrix number 203382_s_at], SEQ ID number 6; CD163 antigen [Affymetrix number 203645_s_at], SEQ ID number 7; phospholipase C, beta 4 [Affymetrix number 203895_at], SEQ ID number 8 or 122; chromosome 11 open reading frame 9 [Affymetrix number 204073_at], SEQ ID number 10; apolipoprotein C-I [Affymetrix number 204416_x_at], SEQ ID number 13; sialyl transferase [Affymetrix number 204542_at], SEQ ID number 14; coagulation factor C homolog, cochlin (Limuluspolyphemus) [Affymetrix number 205229_s_at], SEQ ID number 24; LGN protein [Affymetrix number 205240_at], SEQ ID number 25; peroxisome proliferative activated receptor, gamma [Affymetrix number 208510_s_at], SEQ ID number 41, 136, 137 or 138; allograft inflammatory factor 1 [Affymetrix number 209901_x_at], SEQ ID number 45, 139 or 140; hyaluronoglucosamimidase 1 [Affymetrix number 210619_s_at], SEQ ID number 46, 141, 142, 143, 144, 145, 146 or 147; alpha-1 antitrypsin [Affymetrix number 211429_s_at], SEQ ID number 48, 151 or 152; low density lipoprotein receptor-related protein 4 [Affymetrix number 212850_s_at], SEQ ID number 52; hypothetical protein PP1665 [Affymetrix number 213343_s_at], SEQ ID number 54; serum amyloid A2 [Affymetrix number 214456_x_at], SEQ ID number 60; orosomucoid 2 [Affymetrix number 214465_at], SEQ ID number 61; eyes absent homolog 1 (Drosophila) [Affymetrix number 214608_s_at], SEQ ID number 63, 153, 154 or 155; H factor (complement)-like 1 [Affymetrix number 215388_s_at], SEQ ID number 65; complement component 3 [Affymetrix number 217767_at], SEQ ID number 66; glucosaminyl (N-acetyl) transferase 3, mucin type [Affymetrix number 219508_at], SEQ ID number 68; fascin homolog 1, actin-bundling protein (Strongylocentrotus purpuratus) [Affymetrix number 201564_s)at], SEQ ID number 73; phosphodiesterase 4B, cAMP-specific (phosphodiesterase E4 dunce homolog, Drosophila) [Affymetrix number 203708_at], SEQ ID number 78 or 157; biliverdin reductase A [Affymetrix number 203771_s_at], SEQ ID number 79; IGF-II mRNA-binding protein 3 [Affymetrix number 203819_s_at], SEQ ID number 80; cathepsin E [Affymetrix number 205927_s_at], SEQ ID number 89 or 181; keratin 6A [Affymetrix number 209125_at], SEQ ID number 97, 167 or 168; spondin 1, (f-spondin) extracellular matrix protein [Affymetrix number 209436_at], SEQ ID number 98 or 169; interleukin 1 receptor, type II [Affymetrix number 211372_s_at], SEQ ID number 101, 171 or 172; lipocalin 2 (oncogene 24p3) [Affymetrix number 212531_at], SEQ ID number 104; G protein-coupled receptor 49 [Affymetrix number 213880_at], SEQ ID number 107; spondin 1, (f-spondin) extracellular matrix protein [Affymetrix number 213994_s_at], SEQ ID number 108 or 176; frizzled homolog 10 (Drosophila) [Affymetrix number 219764_at], SEQ ID number 116; deafness locus associated putative guanine nucleotide exchange factor [Affymetrix number 220482_s_at], SEQ ID number 117; CDW52 antigen (CAMPATH-1 antigen) [Affymetrix number 34210_at], SEQ ID number 120.

Accordingly, the invention preferably represents a selection of the 120 represented genes, namely 34, preferably 28 genes, which can be used particularly for determining the responsiveness to 5-FU-containing therapies. Also, in the sense of this invention, the “responsiveness” to a 5-FU-containing chemotherapy/combination therapy determines the expression profile of specific serum markers in blood. These serum markers as well are contained in the 120 marker genes defined herein, and they comprise particularly the following marker genes (and their variants and homologs): complement component 1, q subcomponent, beta polypeptide [Affymetrix number 202953 at], SEQ ID number 4; apolipoprotein E [Affymetrix number 203382_s_at], SEQ ID number 6; apolipoprotein C-I [Affymetrix number 204416_x_at], SEQ ID number 13; coagulation factor V (proaccelerin, labile factor) [Affymetrix number 204714_s_at], SEQ ID number 18; fibrinogen, B beta polypeptide [Affymetrix number 204988_at], SEQ ID number 20; orosomucoid 1 [Affymetrix number 205041_s_at], SEQ ID number 22; apolipoprotein B (including Ag(x) antigen) [Affymetrix number 205108_s_at], SEQ ID number 23; fibrinogen, A alpha polypeptide [Affymetrix number 205650_s_at], SEQ ID number 29; coagulation factor II (thrombin) [Affymetrix number 205754_at], SEQ ID number 30; transferrin [Affymetrix number 214063_s_at], SEQ ID number 58; complement component 4A [Affymetrix number 214428_x_at], SEQ ID number 59; serum amyloid A2 [Affymetrix number 214456_x_at], SEQ ID number 60; orosomucoid 2 [Affymetrix number 214465 at], SEQ ID number 61; complement component 3 [Affymetrix number 217767_at], SEQ ID number 66; complement component 1, q subcomponent, alpha polypeptide [Affymetrix number 218232_at], SEQ ID number 67; fibrinogen, gamma polypeptide [Affymetrix number 219612_s_at], SEQ ID number 69; C-reactive protein, pentraxin-related [Affymetrix number 37020_at], SEQ ID number 71.

One or more (preferably at least two, more preferably at least three) of the described genes, gene fragments, complementary nucleic acids or translated proteins are therefore particularly well suited for predicting the efficacy of a 5-FU-based chemotherapy or combination therapy (5-FU, for example, in combination with folic acid, irinotecan, oxaliplatin and others). In the selection of at least three genes, preferred genes include genes 10, 14, 25, 41, 52, 63, 68 represented in the SEQ ID NOs: chromosome 11 open reading frame 9 [Affymetrix number 204073_s_at], SEQ ID number 10; sialyl transferase [Affymetrix number 204542_at], SEQ ID number 14; LGN protein [Affymetrix number 205240_at], SEQ ID number 25; peroxisome proliferative activated receptor, gamma [Affymetrix number 208510_s_at], SEQ ID number 41, 136, 137 or 138; low density lipoprotein receptor-related protein 4 [Affymetrix number 212850_s_at], SEQ ID number 52; eyes absent homolog 1 (Drosophila) [Affymetrix number 214608_s_at], SEQ ID number 63, 153, 154 or 155; glucosaminyl (N-acetyl) transferase 3, mucin type [Affymetrix number 219508_at], SEQ ID number 68.

### EXAMPLE 16

### Individual Therapy of Liver Metastases of a Colorectal Carcinoma, Derived from the Expression Data of the Respective Patients

Mr. T. is a 42-year-old patient who was diagnosed in July 2003 with a subtotal stenosing sigma carcinoma with liver metastasis. The primary tumor was resected completely (RO) on Jul. 18, 2003, and at the same time a biopsy was carried out from one of the liver metastases. This liver metastasis was subjected to a laser microdissection (according to Examples 1-4), the RNA was isolated and amplified. Then the amplified RNA was hybridized on an Affymetrix chip HG U133-A, and the expression profile was read. The result of the analysis was fixed on Sep. 5, 2003 (see Table 5, column 4), and it yielded a gene signature which predicts a good response (Examples 13-15).

After the surgery, the patient was treated for the entire 2 weeks with a palliative chemotherapy consisting of folic acid and 5-fluorourcil [sic; 5-fluorouracil] by 24-h infusion (AIO regimen), as well as with oxaliplatin. On Aug. 6, 2004, the first cycle of the treatment was started, and at the time of the first check of the tumor response by computer tomography (CT) on Sep. 24, 2003, a response to the chemotherapy was already found. After the completion of the second cycle, a partial remission (a decrease of the size of the liver metastases by >50%) was achieved. Therefore, a third and a fourth cycle of this chemotherapy (CTx) were administered. The provisionally last check of the tumor response took place on Mar. 23, 2004. The reference metastasis has reduced in size from originally (CT finding of Aug. 4, 2003; prior to chemotherapy) 12.9×9.0 cm to now 4.2×7.6 cm (CT finding of Mar. 25, 2004; after 4 chemotherapy cycles) (reduction by 72.6%; corresponds to a partial remission according to WHO criteria). The CT findings of this patient, prior to chemotherapy and after the 4th chemotherapy cycle, can be found in FIG. 5. The sizes of the other liver metastases also regressed, and no new metastases occurred. The patient is alive today (May 2004), and a secondary curative metastasis operation is even under discussion.

The gene expression profiles, as described in Examples 13-15, were tested prospectively on this patient, because the result of the investigations was completed already on Sep. 5, 2004, that is 15 days before the first tumor check under chemotherapy, and the gene expression profile correctly predicted the response to the therapy; see Table 5.

### EXAMPLE 17

### The Validation of the Marker Genes Also Defines SEQ ID NOS: 25, 41 and 68 as Diagnostic Markers for Colorectal Carcinoma

For the additional validation of the above-mentioned results, preoperative biopsies were carried out on the normal colon mucosa and rectum mucosa, respectively, and biopsies of the tumors were removed from an independent set of 12 patients with colorectal carcinoma. After the collection of the biopsies, they were immediately deep frozen in liquid nitrogen and then archived at −80° C.

The samples were then treated as described in Examples 1-3, that is the RNA was extracted from the samples, the RNA was amplified and labeled, and hybridized with HG U133-A Microarrays from Affymtrix [sic; Affymetrix]. The work steps to perform the procedures mentioned were carried out according to the indications given in the Handbook for Gene Expression Analysis (Affymetrix). The signal intensities and the detection signals (“detection calls”) were prepared with the help of the GeneChip 5.0 Software from Affymetrix.

The data of the “minimal sets” of diagnostic markers which are described here for detecting colorectal carcinoma, and which comprise the expression profile of the marker genes defined by Seq ID 25, 41 and 68 and 136-138, were now validated using the above-described set. For this purpose, using the Affy numbers, the corresponding expression values of the 12 mucosa-tumor pairs were determined, and the fold change and the t-test (p<0.05) of the pairs were calculated. As can be seen in Table 6, for the three genes characterized by SEQ NOS: 5, 41 and 68, a significant difference between mucosa and tumor was found. It is worth noting here that of the difference the direction (direction Tu vs Muc) also agrees with Table 2, that is the gene with Seq ID 25 is upregulated in the tumor (compared to the normal tissue), and the gene of Seq ID 41 and 68 is downregulated in the tumor (in comparison to the normal tissue). Thus, it has been shown that the methods described herein, particularly the gene sets characterized herein, and most particularly the “minimal sets” are capable of exactly differentiating tumor from mucosa with a precision of exactly 100% in an independent group of n=12 colorectal carcinomas.

### Tables

TABLE 1
Expression data after statistical evaluation according to Example 4a of responder (Resp)
versus non-responder (Non-Resp) to a 5-FU containing palliative chemotherapy

| \textcircled{1} | | | | Spalte 5 | | Spalte 7 | | |
|---|---|---|---|---|---|---|---|---|
| Spalte 1 | | | Spalte 4 | Fc_Resp | Spalte | Direction | | |
| SEQ | | Spalte 3 | Avg | vs | 6 | Non- | | |
| ID | Spalte 2 | Avg | Non- | Non- | T- | Resp vs | Spalte 8 | Spalte 9 |
| NO: | Affy Nr | Responder | Responder | Resp | Test | Resp | Gene name | Gene |
| 1 | 200954_at | 145.65 | 519.68 | −3.57 | 0.027 | Up | ATPase, H+ transporting, lysosomal 16 kDa, | ATP6V0C |
| | | | | | | | V0 subunit c | |
| 2 | 201137_s_at | 1293.3 | 5177.28 | −4 | 0.045 | Up | major histocompatibility complex, class II, DP | HLA-DPB1 |
| | | | | | | | beta 1 | |
| 3 | 201842_s_at | 871.47 | 3299.13 | −3.79 | 0.029 | Up | EGF-containiag fibulin-like extracellular matrix | EFEMP1 |
| | | | | | | | protein 1 | |
| 4 | 202953_at | 305.67 | 1092.85 | −3.58 | 0.033 | Up | complement component 1, q subcomponent, | C1QB |
| | | | | | | | beta polypeptide | |
| 5 | 202990_at | 174.3 | 768.48 | −4.41 | 0.014 | Up | phosphorylase, glycogen; liver (Hers disease) | PYGL |
| 6 | 203382_s_at | 132.48 | 513.97 | −3.88 | 0.001 | Up | apolipoprotein E | APOE |
| 7 | 203645_s_at | 353.65 | 1385.95 | −3.92 | 0.029 | Up | CD163 antigen | CD163 |
| 8 | 203895_at | 12464.9 | 2658.78 | 4.69 | 0.001 | Down | phospholipase C, beta 4 | PLCB4 |
| 9 | 203939_at | 448.4 | 1686.73 | −3.76 | 0.036 | Up | 5′-nucleotidase, ecto (CD73) | NT5E |
| 10 | 204073_s_at | 359.77 | 1108.27 | −3.08 | 0.029 | Up | chromosome 11 open reading frame 9 | C11orf9 |
| 11 | 204143_s_at | 469.02 | 1854.93 | −3.95 | 0.029 | Up | rTS beta protein | HSRTSBETA |
| 12 | 204204_at | 148.08 | 488.2 | −3.3 | 0.004 | Up | solute carrier family 31 (copper transporters), | SLC31A2 |
| | | | | | | | member 2 | |
| 13 | 204416_x_at | 856.1 | 3347.48 | −3.91 | 0.002 | Up | apolipoprotein C-I | APOCI |
| 14 | 204542_at | 119.03 | 432.17 | −3.63 | 0.038 | Up | sialyltransferase | STHM |
| 15 | 204584_at | 389.25 | 70.62 | 5.51 | 0.014 | Down | L1 cell adhesion molecule (hydrocephalus, | L1CAM |
| | | | | | | | stenosis of aqueduct of Sylvius I) | |
| 16 | 204698_at | 181.85 | 590.4 | −3.25 | 0.027 | Up | interferon stimulated gene 20 kDa | ISG20 |
| 17 | 204705_x_at | 270.88 | 1495.58 | −5.52 | 0.029 | Up | aldolase B, fructose-bisphosphate | ALDOB |
| 18 | 204714_s_at | 231.23 | 1197.85 | −5.18 | 0.005 | Up | coagulation factor V (proaccelerin, labile | F5 |
| | | | | | | | factor) | |
| 19 | 204965_at | 359.55 | 1387.28 | −3.86 | 0.027 | Up | group-specific component (vitamin D binding | GC |
| | | | | | | | protein) | |
| 20 | 204988_at | 2708 | 13973.72 | −5.16 | 0.031 | Up | fibrinogen, B beta polypeptide | FGB |
| 21 | 204992_s_at | 4082.15 | 905.9 | 4.51 | 0.024 | Down | profilin 2 | PFN2 |
| 22 | 205041_s_at | 196.2 | 2513.82 | −12.81 | 0.021 | Up | orosomucoid 1 | ORM1 |
| 23 | 205108_s_at | 209.98 | 845.3 | −4.03 | 0.025 | Up | apolipoprotein B (including Ag(x) antigen) | APOB |
| 24 | 205229_s_at | 804.8 | 209.83 | 3.84 | 0.029 | Down | coagulation factor C homolog, cochlin ( | COCH |
| | | | | | | | Limulus polyphemus) | |
| 25 | 205240_at | 1539.52 | 435.67 | 3.53 | 0.005 | Down | LGN protein | LGN |
| 26 | 205477_s_at | 283.2 | 1155.3 | −4.08 | 0.017 | Up | alpha-1-microglobulin/bikunin precursor | AMBP |
| 27 | 205522_at | 726.27 | 133.53 | 5.44 | 0.005 | Down | homeo box D4 | HOXD4 |
| 28 | 205604_at | 462.38 | 102.25 | 4.52 | 0.022 | Down | homeo box D9 | HOXD9 |
| 29 | 205650_s_at | 493.55 | 2904.28 | −5.88 | 0.041 | Up | fibrinogen. A alpha polypeptide | FGA |
| 30 | 205754_at | 209.75 | 662.38 | −3.16 | 0.026 | Up | coagulation factor II (thrombin) | F2 |
| 31 | 205755_at | 141.5 | 680.27 | −4.81 | 0.037 | Up | pre-alpha (globulin) inhibitor, H3 polypeptide | ITIH3 |
| 32 | 205923_at | 227.28 | 807.67 | −3.55 | 0.02 | Up | reelin | RELN |
| 33 | 205969_at | 175.48 | 757.43 | −4.32 | 0.04 | Up | arylacetamide deacetylase (esterase) | AADAC |
| 34 | 206042_x_at | 896.6 | 212.67 | 4.22 | 0.014 | Down | small nuclear ribonucleoprotein polypeptide N | SNRPN |
| 35 | 206130_s_at | 124.9 | 477.2 | −3.82 | 0.02 | Up | asialoglycoprotein receptor 2 | ASGR2 |
| 36 | 206287_s_at | 24.95 | 372.38 | −14.93 | 0.029 | Up | inter-alpha (globulin) inhibitor H4 (plasma | ITIH4 |
| | | | | | | | Kallikrein-sensitive glycoprotein) | |
| 37 | 206350_at | 555.53 | 2352.27 | −4.23 | 0.038 | Up | amyloid P component, serum | APCS |
| 38 | 207267_s_at | 575.88 | 164.03 | 3.51 | 0.029 | Down | Down syndrome critical region gene 6 | DSCR6 |
| 39 | 207761_s_at | 1638.63 | 5869 | −3.58 | 0.029 | Up | DKFZP586A0522 protein | DKFZP586A0522 |
| 40 | 208470_s_at | 1226.9 | 15875.23 | −12.94 | 0.012 | Up | haptoglobin | HP |
| 41 | 208510_s_at | 3005.18 | 776.35 | 3.87 | 0.023 | Down | peroxisome proliferative activated receptor, | PPARG |
| | | | | | | | gamma | |
| 42 | 208962_s_at | 1947.03 | 556.83 | 3.5 | 0.029 | Down | fatty acid desaturase 1 | FADS1 |
| 43 | 209555_s_at | 74.35 | 315.28 | −4.24 | 0.014 | Up | CD36 antigen (collagen type 1 receptor, | CD36 |
| | | | | | | | thrombospondin receptor) | |
| 44 | 209699_x_at | 709.47 | 3046.28 | −4.29 | 0.014 | Up | aldo-keto reductase family 1, member C2 | AKR1C2 |
| | | | | | | | (dihydrodiol dehydrogenase 2) | |
| 45 | 209901_x_at | 46.32 | 346.83 | −7.49 | 0.042 | Up | allograft inflammatory factor 1 | AIF1 |
| 46 | 210619_s_at | 37.33 | 301.33 | −8.07 | 0.02 | Up | hyaluronoglucosaminidase 1 | HYAL1 |
| 47 | 211367_s_at | 88.02 | 393.8 | −4.47 | 0.029 | Up | caspase 1, apoptosis-related cysteine protease | CASP1 |
| | | | | | | | (ICE-1) | |
| 48 | 211429_s_at | 3552.52 | 19699.27 | −5.55 | 0.017 | Up | alpha-1 Antitrypsin | SERPINA1 |
| 49 | 211991_s_at | 229.8 | 1334.97 | −5.81 | 0.037 | Up | major histocompatibility complex, class II, DP | HLA-DPA1 |
| | | | | | | | alpha 1 | |
| 50 | 212224_at | 141.23 | 716.03 | −5.07 | 0.029 | Up | aldehyde dehydrogenase 1 family, member A1 | ALDH1A1 |
| 51 | 212235_at | 135.98 | 575.18 | −4.23 | 0.013 | Up | plexin D1 | PLXND1 |
| 52 | 212850_s_at | 3434.2 | 1111.52 | 3.09 | 0.043 | Down | low density lipoprotein receptor-related | LRP4 |
| | | | | | | | protein 4 | |
| 53 | 213204_at | 192.18 | 930.65 | −4.84 | 0.029 | Up | p53-associated parkin-like cytoplasmic protein | PARC |
| 54 | 213343_s_at | 419.13 | 119.17 | 3.52 | 0.029 | Down | hypothetical protein PP1665 | PP1665 |
| 55 | 213568_at | 145.08 | 454.45 | −3.13 | 0.037 | Up | odd-skipped-related 2A protein | OSR2 |
| 56 | 213592_at | 329.2 | 1239.5 | −3.77 | 0.017 | Up | angiotensin II receptor-like 1 | AGTRL1 |
| 57 | 213988_s_at | 1016.7 | 3291.8 | −3.24 | 0.047 | Up | spermidine/spermine N1-acetyltransferase | SAT |
| 58 | 214063_s_at | 237.9 | 1677.93 | −7.05 | 0.046 | Up | transferrin | TF |
| 59 | 214428_x_at | 779.75 | 2975.48 | −3.82 | 0.005 | Up | complement component 4A | C4A |
| 60 | 214456_x_at | 264.88 | 4909.38 | −18.53 | 0.038 | Up | serum amyloid A2 | SAA2 |
| 61 | 214465_at | 66.25 | 762.43 | −11.51 | 0.026 | Up | orosomucoid 2 | ORM2 |
| 62 | 214604_at | 1215.43 | 131.85 | 9.22 | 0.005 | Down | homeo box D11 | HOXD11 |
| 63 | 214608_s_at | 572.88 | 28.28 | 20.26 | 0.014 | Down | eyes absent homolog 1 (Drosophila) | EYA1 |
| 64 | 214669_x_at | 200.98 | 624.15 | −3.11 | 0.038 | Up | similar to Human Ig rearranged gamma chain | na |
| | | | | | | | mRNA, V-J-C region | |
| 65 | 215388_s_at | 336.75 | 1303.15 | −3.87 | 0.021 | Up | H factor (complement)-like 1 | HFL1 |
| 66 | 217767_at | 953.25 | 5588.37 | −5.86 | 0.038 | Up | complement component 3 | C3 |
| 67 | 218232_at | 93.93 | 390.22 | −4.15 | 0.005 | Up | complement component 1, q subcomponent, | C1QA |
| | | | | | | | alpha polypeptide | |
| 68 | 219508_at | 2260.57 | 9751.88 | −4.31 | 0.046 | Up | glucosaminyl (N-acetyl) transferase 3, mucin | GCNT3 |
| | | | | | | | type | |
| 69 | 219612_s_at | 1133.45 | 7741.42 | −6.83 | 0.038 | Up | fibrinogen, gamma polypeptide | FGG |
| 70 | 222162_s_at | 183.68 | 646.53 | −3.52 | 0.029 | Up | a disintegrin-like and metalloprotease with | ADAMTS1 |
| | | | | | | | thrombospondin type 1 | |
| 71 | 37020_at | 462.3 | 3024.4 | −6.54 | 0.029 | Up | C-reactive protein, pentraxin-related | CRP |
| 72 | 39763_at | 185.4 | 988.95 | −5.33 | 0.021 | Up | hemopexin | HPX |

Key: \textcircled{1} Column
Description for Table 1
Table 1 lists all 72 genes which are relevant, after the evaluation of the raw data according to the statistical procedure from Example 4a, for a comparison of the gene expression of responders (Resp) with that of non-responders (Non-Resp) in reference to a response to a 5-FU-containing palliative chemotherapy in metastasized colorectal carcinoma.
Column 1 contains the SEQ ID NOS of the gene
Column 2 contains the Affymetrix Accession Numbers on the HG U-133-A Microarray
Column 3 contains the mean values of the gene expression of (n = 8) responders
Column 4 contains the mean values of the gene expression of (n = 9) non-responders
Column 5 contains the differences in the gene expression between responders and non-responders, indicated as fold-change (Fc)
Column 6 shows the significance level in the t-test
Column 7 shows whether the individual genes in the non-responders have been upregulated (up) or downregulated (down) (in comparison to the responders)
Column 8 contains the names of the genes
Column 9 contains the gene symbol

TABLE 2
Expression data colon mucosa (Muc) versus colorectal primary tumor (Tu) versus liver
metastasis (Lm) after statistical evaluation according to Example 4a

| \textcircled{1} | | | | | | Spalte 7 | |
|---|---|---|---|---|---|---|---|
| Spalte 1 | | | | | Spalte 6 | T- | Spalte 8 |
| SEQ | | Spalte 3 | Spalte 4 | Spalte 5 | Fc_Tu | Test | Direction |
| ID | Spalte 2 | Avg | Avg | Avg | vs | Tu vs | Tu vs |
| NO:. | Affy Nr | Mucosa | Tumor | Metastase | Muc | Muc | Muc |
| 1 | 200954_at | 408.70 | 372.73 | 397.40 | 1.10 | 0.839 | None |
| 2 | 201137_s_at | 17222.77 | 3310.17 | 2739.40 | 5.20 | 0.021 | Down |
| 3 | 201842_s_at | 5613.23 | 2293.00 | 1782.83 | 2.45 | 0.205 | None |
| 4 | 202953_at | 2334.17 | 794.47 | 600.64 | 2.94 | 0.103 | None |
| 5 | 202990_at | 572.30 | 426.02 | 533.91 | 1.34 | 0.369 | None |
| 6 | 203382_s_at | 498.80 | 249.17 | 290.08 | 2.00 | 0.241 | None |
| 7 | 203645_s_at | 2161.43 | 910.36 | 895.28 | 2.37 | 0.005 | Down |
| 8 | 203895_at | 2763.03 | 8833.41 | 7288.26 | −3.20 | 0.048 | Up |
| 9 | 203939_at | 1591.57 | 1376.01 | 1061.64 | 1.16 | 0.632 | None |
| 10 | 204073_s_at | 449.53 | 441.00 | 703.39 | 1.02 | 0.950 | None |
| 11 | 204143_s_at | 1458.70 | 956.46 | 1467.93 | 1.53 | 0.287 | None |
| 12 | 204204_at | 827.57 | 438.22 | 327.87 | 1.89 | 0.011 | Down |
| 13 | 204416_x_at | 974.17 | 915.77 | 1945.07 | 1.06 | 0.882 | None |
| 14 | 204542_at | 258.77 | 218.93 | 224.52 | 1.18 | 0.742 | None |
| 15 | 204584_at | 250.57 | 118.13 | 181.68 | 2.12 | 0.191 | None |
| 16 | 204698_at | 1465.67 | 242.37 | 310.35 | 6.05 | 0.197 | None |
| 17 | 204705_x_at | 567.47 | 265.38 | 869.25 | 2.14 | 0.541 | None |
| 18 | 204714_s_at | 215.23 | 264.31 | 650.14 | −1.23 | 0.678 | None |
| 19 | 204965_at | 124.07 | 63.37 | 925.61 | 1.96 | 0.011 | Down |
| 20 | 204988_at | 182.17 | 87.33 | 7336.73 | 2.09 | 0.589 | None |
| 21 | 204992_s_at | 2196.70 | 3489.70 | 3119.97 | −1.59 | 0.047 | Up |
| 22 | 205041_s_at | 47.77 | 58.01 | 1752.03 | −1.21 | 0.727 | None |
| 23 | 205108_s_at | 142.97 | 62.90 | 834.18 | 2.27 | 0.106 | None |
| 24 | 205229_s_at | 364.97 | 926.46 | 856.14 | −2.54 | 0.253 | None |
| 25 | 205240_at | 279.40 | 1207.46 | 1139.79 | −4.32 | 0.005 | Up |
| 26 | 205477_s_at | 46.23 | 34.97 | 701.76 | 1.32 | 0.772 | None |
| 27 | 205522_at | 640.93 | 368.34 | 295.40 | 1.74 | 0.075 | None |
| 28 | 205604_at | 154.23 | 231.71 | 226.38 | −1.50 | 0.068 | None |
| 29 | 205650_s_at | 178.17 | 140.14 | 1761.42 | 1.27 | 0.432 | None |
| 30 | 205754_at | 22.73 | 55.83 | 415.07 | −2.46 | 0.152 | None |
| 31 | 205755_at | 29.30 | 38.10 | 422.89 | −1.30 | 0.552 | None |
| 32 | 205923_at | 657.37 | 315.72 | 455.40 | 2.08 | 0.072 | None |
| 33 | 205969_at | 25.07 | 125.41 | 511.29 | −5.00 | 0.030 | Up |
| 34 | 206042_x_at | 311.03 | 264.57 | 396.37 | 1.18 | 0.517 | None |
| 35 | 206130_s_at | 43.47 | 57.18 | 271.50 | −1.32 | 0.319 | None |
| 36 | 206287_s_at | 26.57 | 16.36 | 192.48 | 1.33 | 0.065 | None |
| 37 | 206350_at | 195.43 | 163.91 | 1249.63 | 1.19 | 0.395 | None |
| 38 | 207267_s_at | 45.70 | 232.96 | 278.08 | −5.10 | 0.000 | Up |
| 39 | 207761_s_at | 15860.73 | 4047.53 | 3121.27 | 3.92 | 0.080 | None |
| 40 | 208470_s_at | 222.47 | 71.82 | 7626.67 | 3.10 | 0.416 | None |
| 41 | 208510_s_at | 2608.67 | 1188.71 | 1540.47 | 2.19 | 0.040 | Down |
| 42 | 208962_s_at | 483.53 | 1396.28 | 837.45 | −2.89 | 0.050 | Up |
| 43 | 209555_s_at | 1135.73 | 193.56 | 220.56 | 5.87 | 0.098 | None |
| 44 | 209699_x_at | 3171.57 | 1635.38 | 2308.79 | 1.94 | 0.348 | None |
| 45 | 209901_x_at | 552.10 | 181.39 | 155.06 | 3.04 | 0.012 | Down |
| 46 | 210619_s_at | 60.00 | 86.60 | 120.30 | −1.44 | 0.438 | None |
| 47 | 211367_s_at | 926.10 | 432.47 | 243.66 | 2.14 | 0.050 | Down |
| 48 | 211429_s_at | 2295.53 | 2216.16 | 9488.42 | 1.04 | 0.902 | None |
| 49 | 211991_s_at | 6946.27 | 972.13 | 676.73 | 7.15 | 0.021 | Down |
| 50 | 212224_at | 1218.77 | 450.14 | 622.11 | 2.71 | 0.010 | Down |
| 51 | 212235_at | 84.63 | 350.34 | 354.51 | −4.14 | 0.000 | Up |
| 52 | 212850_s_at | 890.13 | 1750.36 | 2601.93 | −1.97 | 0.162 | None |
| 53 | 213204_at | 196.90 | 255.64 | 446.46 | −1.30 | 0.623 | None |
| 54 | 213343_s_at | 15.47 | 125.19 | 173.24 | −6.26 | 0.005 | Up |
| 55 | 213568_at | 360.83 | 321.92 | 254.19 | 1.12 | 0.730 | None |
| 56 | 213592_at | 769.43 | 865.09 | 699.26 | −1.12 | 0.689 | None |
| 57 | 213988_s_at | 4964.47 | 3437.08 | 2846.43 | 1.44 | 0.340 | None |
| 58 | 214063_s_at | 142.40 | 61.62 | 987.28 | 2.31 | 0.338 | None |
| 59 | 214428_x_at | 906.10 | 552.37 | 1554.22 | 1.64 | 0.172 | None |
| 60 | 214456_x_at | 276.03 | 589.09 | 3362.99 | −2.13 | 0.185 | None |
| 61 | 214465_at | 28.50 | 29.79 | 497.97 | −1.05 | 0.891 | None |
| 62 | 214604_at | 216.97 | 314.54 | 453.00 | −1.45 | 0.144 | None |
| 63 | 214608_s_at | 42.57 | 259.89 | 148.71 | −6.11 | 0.272 | None |
| 64 | 214669_x_at | 15563.97 | 1369.14 | 413.48 | 11.37 | 0.062 | None |
| 65 | 215388_s_at | 996.20 | 448.00 | 901.10 | 2.22 | 0.163 | None |
| 66 | 217767_at | 1413.73 | 785.02 | 3880.91 | 1.80 | 0.006 | Down |
| 67 | 218232_at | 933.40 | 349.86 | 219.99 | 2.67 | 0.065 | None |
| 68 | 219508_at | 15840.80 | 3753.78 | 6111.93 | 4.22 | 0.000 | Down |
| 69 | 219612_s_at | 158.60 | 56.27 | 4577.00 | 2.82 | 0.273 | None |
| 70 | 222162_s_at | 2005.40 | 494.23 | 357.27 | 4.06 | 0.015 | Down |
| 71 | 37020_at | 8.43 | 8.97 | 1678.06 | −0.45 | 0.674 | None |
| 72 | 39763_at | 155.00 | 113.86 | 626.34 | 1.36 | 0.139 | None |

| | | Spalte | | | |
|---|---|---|---|---|---|
| \textcircled{1} | | 10 | | | |
| Spalte 1 | | T- | Spalte 11 | | |
| SEQ | Spalte 9 | Test | Direction | | |
| ID | Fc_Lm | Lm | Lm vs | Spalte 12 | Spalte 13 |
| NO:. | vs Tu | vs Tu | Tu | Gene name | Gene |
| 1 | −1.07 | 0.821 | None | ATPase, H+ transporting, | ATP6V0C |
| | | | | lysosomal 16 kDa, V0 | |
| | | | | subunit c | |
| 2 | 1.21 | 0.362 | None | major histocompatibility | HLA-DPB1 |
| | | | | complex, class II, DP beta 1 | |
| 3 | 1.29 | 0.114 | None | EGF-containing fibulin- | EFEMP1 |
| | | | | like extracellular matrix | |
| | | | | protein 1 | |
| 4 | 1.32 | 0.339 | None | complement component 1, | C1QB |
| | | | | q subcomponent, beta | |
| | | | | polypeptide | |
| 5 | −1.25 | 0.502 | None | phosphorylase, glycogen; | PYGL |
| | | | | liver (Hers disease) | |
| 6 | −1.16 | 0.774 | None | apolipoprotein E | APOE |
| 7 | 1.02 | 0.557 | None | CD163 antigen | CD163 |
| 8 | 1.21 | 0.691 | None | phospholipase C, beta 4 | PLCB4 |
| 9 | 1.30 | 0.411 | None | 5′-nucleotidase, ecto | NT5E |
| | | | | (CD73) | |
| 10 | −1.59 | 0.229 | None | chromosome 11 open | C11orf9 |
| | | | | reading frame 9 | |
| 11 | −1.53 | 0.206 | None | rTS beta protein | HSRTSBETA |
| 12 | 1.34 | 0.192 | None | solute carrier family 31 | SLC31A2 |
| | | | | (copper transporters), | |
| | | | | member 2 | |
| 13 | −2.12 | 0.076 | None | apolipoprotein C-I | APOCI |
| 14 | −1.03 | 0.925 | None | sialyltransferase | STHM |
| 15 | −1.54 | 0.252 | None | L1 cell adhesion molecule | LICAM |
| | | | | (hydrocephalus, stenosis | |
| | | | | of aqueduct of Sylvius 1) | |
| 16 | −1.28 | 0.702 | None | interferon stimulated gene | ISG20 |
| | | | | 20 kDa | |
| 17 | −3.28 | 0.047 | Up | aldolase B, fructose- | ALDOB |
| | | | | bisphosphate | |
| 18 | −2.46 | 0.171 | None | coagulation factor V | F5 |
| | | | | (proaccelerin, labile | |
| | | | | factor) | |
| 19 | −14.61 | 0.016 | Up | group-specific component | GC |
| | | | | (vitamin D binding | |
| | | | | protein) | |
| 20 | −84.01 | 0.002 | Up | fibrinogen, B beta | FGB |
| | | | | polypeptide | |
| 21 | 1.12 | 0.728 | None | profilin 2 | PFN2 |
| 22 | −30.20 | 0.041 | Up | orosomucoid 1 | ORM1 |
| 23 | −13.26 | 0.106 | None | apolipoprotein B | APOB |
| | | | | (including Ag(x) antigen) | |
| 24 | 1.08 | 0.928 | None | coagulation factor C | COCH |
| | | | | homolog, cochlin | |
| | | | | (Limulus polyphemus) | |
| 25 | 1.06 | 0.906 | None | LGN protein | LGN |
| 26 | −20.07 | 0.012 | Up | alpha-1. | AMBP |
| | | | | microglobulin/bikunin | |
| | | | | precursor | |
| 27 | 1.25 | 0.477 | None | homeo box D4 | HOXD4 |
| 28 | 1.02 | 1.000 | None | homeo box D9 | HOXD9 |
| 29 | −12.57 | 0.023 | Up | fibrinogen, A alpha | FGA |
| | | | | polypeptide | |
| 30 | −7.43 | 0.003 | Up | coagulation factor II | F2 |
| | | | | (thrombin) | |
| 31 | −11.10 | 0.020 | Up | pre-alpha (globulin) | 1TIH3 |
| | | | | inhibitor, H3 polypeptide | |
| 32 | −1.44 | 0.364 | None | reelin | RELN |
| 33 | −4.08 | 0.003 | Up | arylacetamide deacetylase | AADAC |
| | | | | (esterase) | |
| 34 | −1.50 | 0.193 | None | small nuclear | SNRPN |
| | | | | ribonucleoprotein | |
| | | | | polypeptide N | |
| 35 | −4.75 | 0.005 | Up | asialoglycoprotein | ASGR2 |
| | | | | receptor 2 | |
| 36 | −9.62 | 0.050 | None | inter-alpha (globulin) | ITIH4 |
| | | | | inhibitor H4 (plasma | |
| | | | | Kallikrein-sensitive | |
| | | | | glycoprotein) | |
| 37 | −7.62 | 0.009 | Up | amylopid P component, | APCS |
| | | | | serum | |
| 38 | −1.19 | 0.366 | None | Down syndrome critical | DSCR6 |
| | | | | region gene 6 | |
| 39 | 1.30 | 0.391 | None | DKFZP586A0522 protein | DKFZP586A0$$ |
| 40 | −106.19 | 0.004 | Up | haptoglobin | HP |
| 41 | −1.30 | 0.311 | None | peroxisome proliferative | PPARG |
| | | | | activated receptor, gamma | |
| 42 | 1.67 | 0.231 | None | fatty acid desaturase 1 | FADS1 |
| 43 | −1.14 | 0.886 | None | CD36 antigen (collagen | CD36 |
| | | | | type I receptor, | |
| | | | | thrombospondin receptor) | |
| 44 | −1.41 | 0.531 | None | aldo-keto reductase family | AKR1C2 |
| | | | | 1, member C2 | |
| | | | | (dihydrodiol | |
| | | | | dehydrogenase 2) | |
| 45 | 1.17 | 0.593 | None | allograft inflammatory | AIF1 |
| | | | | factor 1 | |
| 46 | −1.39 | 0.459 | None | hyaluronoglucosaminidse 1 | HYAL1 |
| 47 | 1.77 | 0.271 | None | caspase 1, apoptosis- | CASP1 |
| | | | | related cysteine protease | |
| | | | | (ICE-1) | |
| 48 | −4.28 | 0.008 | Up | alpha-1 Antitrypsin | SERPINA1 |
| 49 | 1.44 | 0.182 | None | major histocompatibility | HLA-DPA1 |
| | | | | complex, class II, DP | |
| | | | | alpha 1 | |
| 50 | −1.38 | 0.424 | None | aldehyde dehydrogenase 1 | ALDH1A1 |
| | | | | family, member A1 | |
| 51 | −1.01 | 0.905 | None | plexin D1 | PLXND1 |
| 52 | −1.49 | 0.325 | None | low density lipoprotein | LRP4 |
| | | | | receptor-related protein 4 | |
| 53 | −1.75 | 0.232 | None | p53-associated parkin-like | PARC |
| | | | | cytoplasmic protein | |
| 54 | −1.38 | 0.292 | None | hypothetical protein | PP1665 |
| | | | | PP1665 | |
| 55 | 1.27 | 0.519 | None | odd-skipped-related 2A | OSR2 |
| | | | | protein | |
| 56 | 1.24 | 0.308 | None | angiotensin II receptor- | AGTRL1 |
| | | | | like 1 | |
| 57 | 1.21 | 0.407 | None | spermidine/spermine N1- | SAT |
| | | | | acetyltransferase | |
| 58 | −16.02 | 0.028 | Up | transferrin | TF |
| 59 | −2.81 | 0.021 | Up | complement component | C4A |
| | | | | 4A | |
| 60 | −5.71 | 0.100 | None | serum amyloid A2 | SAA2 |
| 61 | −16.72 | 0.052 | None | orosomucoid 2 | ORM2 |
| 62 | −1.44 | 0.307 | None | homeo box D11 | HOXD11 |
| 63 | 1.75 | 0.617 | None | eyes absent homolog 1 | EYA1 |
| | | | | (Drosophila) | |
| 64 | 3.31 | 0.014 | Down | similar to Human Ig | na |
| | | | | rearranged gamma chain | |
| | | | | mRNA, V-J-C region | |
| 65 | −2.01 | 0.149 | None | H factor (complement)- | HFL1 |
| | | | | like 1 | |
| 66 | −4.94 | 0.029 | Up | complement component 3 | C3 |
| 67 | 1.59 | 0.236 | None | complement component 1, | C1QA |
| | | | | q subcomponent, alpha | |
| | | | | polypeptide | |
| 68 | −1.63 | 0.261 | None | glucosaminyl (N-acetyl) | GCNT3 |
| | | | | transferase 3, mucin type | |
| 69 | −81.34 | 0.004 | Up | fibrinogen, gamma | FGG |
| | | | | polypeptide | |
| 70 | 1.38 | 0.208 | None | a disintegrin-like and | ADAMTS1 |
| | | | | metalloprotease with | |
| | | | | thrombospondin type 1 | |
| 71 | −83.90 | 0.026 | Up | C-reactive protein, | CRP |
| | | | | pentraxin-related | |
| 72 | −5.50 | 0.024 | Up | hemopexin | HPX |

Key: \textcircled{1} Column
Description for Table 2
Table 2 lists all 72 genes which, after the evaluation of the raw data according to the statistical procedure from Example 4a, are relevant for a comparison of the gene expression between normal tissue (normal colon mucosa (Muc)), colorectal primary tumor (Tu) and liver metastasis (Lm) of these colorectal carcinomas.
Column 1 contains the SEQ ID NOS of the gene
Column 2 contains the Affymetrix Accession Numbers on the HG U-133-A Microarray
Column 3 contains the mean values of the gene expression of (n = 3) normal mucosa
Column 4 contains the mean values of the gene expression of (n = 10) colorectal primary tumors
Column 5 contains the mean values of the gene expression of (n = 20) liver metastases (corresponding with colorectal carcinomas)
Column 6 contains the differences in gene expression between tumor and normal tissue indicated as fold-change (Fc)
Column 7 shows the significance level in the t-test
Column 8 shows whether the individual tissues have been upregulated (up) or downregulated (down) or are not significantly different (none) (in comparison to the normal tissue)
Column 9 contains the different in gene expression between liver metastasis and tumor indicated as fold-change (Fc)
Column 10 shows the significance level in the t-test
Column 11 shows whether the individual genes in the liver metastasis have been upregulated (up) or downregulated (down) or are not significantly different (none) (in comparison to the tumor)
Column 12 contains the names of the genes
Column 13 contains the gene symbol

TABLE 3
Expression data colon mucosa (Muc) versus colorectal primary tumor (Tu) versus liver
metastasis (Lm) after statistical evaluation according to Example 4b

| \textcircled{1} | | | | | | Spalte 7 | |
|---|---|---|---|---|---|---|---|
| Spalte 1 | | | | | Spalte 6 | T- | Spalte 8 |
| SEQ | | Spalte 3 | Spalte 4 | Spalte 5 | Fc_Tu | Test | Direction |
| ID | Spalte 2 | Avg | Avg | Avg | vs | Tu vs | Tu vs |
| NO: | Affy Nr. | Mucosa | Tumor | Metastase | Muc | Muc | Muc |
| 73 | 201564_s_at | 106.20 | 299.57 | 173.81 | −2.82 | 0.054 | None |
| 74 | 201645_at | 503.17 | 1016.04 | 463.12 | −2.02 | 0.014 | Up |
| 75 | 202973_x_at | 4621.03 | 3570.39 | 4282.19 | 1.29 | 0.521 | None |
| 76 | 203180_at | 729.80 | 1381.29 | 758.24 | −1.89 | 0.071 | None |
| 77 | 203649_s_at | 686.60 | 400.81 | 231.62 | 1.71 | 0.075 | None |
| 78 | 203708_at | 1319.57 | 750.28 | 492.61 | 1.76 | 0.043 | Down |
| 79 | 203771_s_at | 304.17 | 100.96 | 119.50 | 3.01 | 0.021 | Down |
| 80 | 203819_s_at | 63.50 | 183.51 | 790.74 | −2.89 | 0.167 | None |
| 81 | 203837_at | 1250.93 | 2099.21 | 1639.14 | −1.68 | 0.092 | None |
| 10 | 204073_s_at | 449.53 | 441.00 | 703.39 | 1.02 | 0.950 | None |
| 14 | 204542_at | 258.77 | 218.93 | 224.52 | 1.18 | 0.742 | None |
| 82 | 204673_at | 8751.97 | 1551.27 | 1509.46 | 5.64 | 0.000 | Down |
| 83 | 205097_at | 20565.07 | 1669.43 | 845.78 | 12.32 | 0.012 | Down |
| 25 | 205240_at | 279.40 | 1207.46 | 1139.79 | −4.32 | 0.005 | Up |
| 84 | 205278_at | 42.83 | 160.86 | 240.36 | −3.76 | 0.218 | None |
| 85 | 205372_at | 134.47 | 349.76 | 490.25 | −2.60 | 0.008 | Up |
| 86 | 205414_s_at | 1216.53 | 307.57 | 456.31 | 3.96 | 0.000 | Down |
| 87 | 205767_at | 228.30 | 925.02 | 995.84 | −4.05 | 0.151 | None |
| 88 | 205865_at | 141.40 | 222.88 | 223.25 | −1.58 | 0.080 | None |
| 89 | 205927_s_at | 2517.43 | 3476.27 | 2453.84 | −1.38 | 0.750 | None |
| 90 | 205937_at | 68.63 | 170.00 | 214.15 | −2.48 | 0.065 | None |
| 91 | 206884_s_at | 32.90 | 143.10 | 183.25 | −4.35 | 0.301 | None |
| 92 | 207011_s_at | 109.60 | 277.71 | 167.70 | −2.53 | 0.035 | Up |
| 93 | 207039_at | 134.83 | 339.42 | 549.34 | −2.52 | 0.073 | None |
| 94 | 207063_at | 90.93 | 166.79 | 172.44 | −1.83 | 0.486 | None |
| 95 | 207509_s_at | 126.00 | 256.13 | 165.25 | −2.03 | 0.148 | None |
| 96 | 208262_x_at | 113.20 | 76.39 | 63.70 | 1.48 | 0.498 | None |
| 41 | 208510_s_at | 2608.67 | 1188.71 | 1540.47 | 2.19 | 0.040 | Down |
| 97 | 209125_at | 108.57 | 242.70 | 1902.08 | −2.24 | 0.168 | None |
| 98 | 209436_at | 5056.93 | 2371.10 | 1757.15 | 2.13 | 0.071 | None |
| 99 | 209905_at | 1536.53 | 4320.54 | 4507.15 | −2.81 | 0.014 | Up |
| 100 | 210164_at | 209.77 | 985.48 | 1052.44 | −4.70 | 0.096 | None |
| 101 | 211372_s_at | 2553.83 | 321.82 | 278.83 | 7.94 | 0.013 | Down |
| 102 | 211430_s_at | 5355.93 | 3219.96 | 1384.60 | 1.66 | 0.335 | None |
| 103 | 212349_at | 279.23 | 789.60 | 840.55 | −2.83 | 0.020 | Up |
| 104 | 212531_at | 741.50 | 10783.23 | 5421.20 | −14.54 | 0.008 | Up |
| 52 | 212850_s_at | 890.13 | 1750.36 | 2601.93 | −1.97 | 0.162 | None |
| 105 | 213230_at | 72.20 | 362.67 | 368.39 | −5.02 | 0.008 | Up |
| 106 | 213435_at | 5470.87 | 4275.04 | 3364.76 | 1.28 | 0.226 | None |
| 107 | 213880_at | 186.80 | 3407.68 | 1647.43 | −18.24 | 0.062 | None |
| 108 | 213994_s_at | 1297.73 | 871.50 | 570.75 | 1.49 | 0.409 | None |
| 63 | 214608_s_at | 42.57 | 259.89 | 148.71 | −6.11 | 0.272 | None |
| 109 | 214677_x_at | 41827.57 | 5890.19 | 1491.95 | 7.10 | 0.009 | Down |
| 110 | 216910_at | 133.67 | 55.04 | 70.65 | 2.43 | 0.149 | None |
| 111 | 217047_s_at | 2877.57 | 2235.86 | 2657.61 | 1.29 | 0.606 | None |
| 112 | 217988_at | 1552.83 | 2601.08 | 3201.26 | −1.68 | 0.025 | Up |
| 113 | 218445_at | 275.27 | 539.87 | 475.70 | −1.96 | 0.157 | None |
| 68 | 219508_at | 15840.80 | 3753.78 | 6111.93 | 4.22 | 0.000 | Down |
| 114 | 219697_at | 452.87 | 223.17 | 244.57 | 2.03 | 0.010 | Down |
| 115 | 219735_s_at | 1806.57 | 1183.51 | 943.86 | 1.53 | 0.029 | Down |
| 116 | 219764_at | 119.33 | 672.81 | 464.35 | −5.64 | 0.135 | None |
| 117 | 220482_s_at | 165.07 | 131.71 | 196.26 | 1.25 | 0.611 | None |
| 118 | 220658_s_at | 70.40 | 222.04 | 291.74 | −3.15 | 0.003 | Up |
| 119 | 222347_at | 172.97 | 115.60 | 221.98 | 1.50 | 0.451 | None |
| 120 | 34210_at | 1209.80 | 470.02 | 536.68 | 2.57 | 0.062 | None |

| | | Spalte | | | | |
|---|---|---|---|---|---|---|
| \textcircled{1} | | 10 | | | | |
| Spalte 1 | | T- | Spalte 11 | | | |
| SEQ | Spalte 9 | Test | Direction | | | |
| ID | Fc_Lm | Lm | Lm vs | Spalte 12 | Spalte13 | |
| NO: | vs Tu | vs Tu | Tu | Gene Name | Gene | Spalte 14 |
| 73 | 1.72 | 0.214 | None | fascin homolog 1, | FSCN1 | |
| | | | | actin-bundling | | |
| | | | | protein | | |
| | | | | (Strongylocentrotus | | |
| | | | | purpuratus) | | |
| 74 | 2.19 | 0.005 | Down | tenascin C | TNC | |
| | | | | (hexabrachion) | | |
| 75 | −1.20 | 0.512 | None | family with | FAM13A1 | |
| | | | | sequence similarity | | |
| | | | | 13, member A1 | | |
| 76 | 1.82 | 0.054 | None | aldehyde | ALDH1A3 | |
| | | | | dehydrogenase 1 | | |
| | | | | family, member A3 | | |
| 77 | 1.73 | 0.282 | None | phospholipase A2, | PLA2G2A | |
| | | | | group IIA | | |
| | | | | (platelets, synovial | | |
| | | | | fluid) | | |
| 78 | 1.52 | 0.313 | None | phosphodiesterase | PDE4B | |
| | | | | 4B, cAMP-specific | | |
| | | | | (phosphodiesterase | | |
| | | | | E4 dunce homolog, | | |
| | | | | Drosophila) | | |
| 79 | −1.18 | 0.996 | None | biliverdin reductase A | BLVRA | |
| 80 | −4.31 | 0.066 | None | IGF-II mRNA- | IMP-3 | |
| | | | | binding protein 3 | | |
| 81 | 1.28 | 0.345 | None | mitogen-activated | MAP3K5 | |
| | | | | protein kinase | | |
| | | | | kinase kinase 5 | | |
| 10 | −1.59 | 0.229 | None | chromosome 11 | C11orf9 | x |
| | | | | open reading frame 9 | | |
| 14 | −1.03 | 0.925 | None | sialyltransferase | STHM | x |
| 82 | 1.03 | 0.927 | None | mucin 2, | MUC2 | |
| | | | | intestinal/tracheal | | |
| | | | | /// mucin 2, | | |
| | | | | intestinal/tracheal | | |
| 83 | 1.97 | 0.420 | None | solute carrier | SLC26A2 | |
| | | | | family 26 (sulfate | | |
| | | | | transporter), | | |
| | | | | member 2 | | |
| 25 | 1.06 | 0.906 | None | LGN protein | LGN | x |
| 84 | −1.49 | 0.630 | None | glutamate | GAD1 | |
| | | | | decarboxylase 1 | | |
| | | | | (brain, 67 kDa) | | |
| 85 | −1.40 | 0.228 | None | pleiomorphic | PLAG1 | |
| | | | | adenoma gene 1 | | |
| 86 | −1.48 | 0.207 | None | KIAA0672 gene | KIAA0672 | |
| | | | | product | | |
| 87 | −1.08 | 0.861 | None | epiregulin | EREG | |
| 88 | −1.00 | 0.856 | None | dead ringer-like 1 | DRIL1 | |
| | | | | (Drosophila) | | |
| 89 | 1.42 | 0.710 | None | cathepsin E | CTSE | |
| 90 | −1.26 | 0.435 | None | cell growth | CGREF1 | |
| | | | | regulator with EF | | |
| | | | | hand domain 1 | | |
| 91 | −1.28 | 0.722 | None | sciellin | SCEL | |
| 92 | 1.66 | 0.003 | Down | PTK7 protein | PTK7 | |
| | | | | tyrosine kinase 7 | | |
| 93 | −1.62 | 0.253 | None | cyclin-dependent | CDKN2A | |
| | | | | kinase inhibitor 2A | | |
| | | | | (melanoma, p16, | | |
| | | | | inhibits CDK4) | | |
| 94 | −1.03 | 0.897 | None | chromosome Y | CYorf14 | |
| | | | | open reading frame | | |
| | | | | 14 | | |
| 95 | 1.55 | 0.273 | None | leukocyte- | LAIR2 | |
| | | | | associated Ig-like | | |
| | | | | receptor 2 | | |
| 96 | 1.20 | 0.662 | None | Mediterranean | MEFV | |
| | | | | fever | | |
| 41 | −1.30 | 0.311 | None | peroxisome | PPARG | x |
| | | | | proliferative | | |
| | | | | activated receptor, | | |
| | | | | gamma | | |
| 97 | −7.84 | 0.313 | None | keratin 6A | KRT6A | |
| 98 | 1.35 | 0.536 | None | spondin 1, (f- | SPON1 | |
| | | | | spondin) | | |
| | | | | extracellular matrix | | |
| | | | | protein | | |
| 99 | −1.04 | 0.718 | None | homeo box A9 | HOXA9 | |
| 100 | −1.07 | 0.863 | None | granzyme B | GZMB | |
| | | | | (granzyme 2, | | |
| | | | | cytotoxic T- | | |
| | | | | lymphocyte- | | |
| | | | | associated serine | | |
| | | | | esterase 1) | | |
| 101 | 1.15 | 0.649 | None | interleukin 1 | IL1R2 | |
| | | | | receptor, type II | | |
| 102 | 2.33 | 0.099 | None | immunoglobulin | IGHG3 | |
| | | | | heavy constant | | |
| | | | | gamma 3 (G3m | | |
| | | | | marker) | | |
| 103 | −1.06 | 0.685 | None | protein O- | POFUT1 | |
| | | | | fucosyltransferase 1 | | |
| 104 | 1.99 | 0.111 | None | lipocalin 2 | LCN2 | |
| | | | | (oncogene 24p3) | | |
| 52 | −1.49 | 0.325 | None | low density | LRP4 | x |
| | | | | lipoprotein | | |
| | | | | receptor-related | | |
| | | | | protein 4 | | |
| 105 | −1.02 | 0.885 | None | parancoplastic | HUMPPA | |
| | | | | antigen | | |
| 106 | 1.27 | 0.432 | None | SATB family | SATB2 | |
| | | | | member 2 | | |
| 107 | 2.07 | 0.305 | None | G protein-coupled | GPR49 | |
| | | | | receptor 49 | | |
| 108 | 1.53 | 0.508 | None | spondin 1, (f- | SPON1 | |
| | | | | spondin) | | |
| | | | | extracellular matrix | | |
| | | | | protein | | |
| 63 | 1.75 | 0.617 | None | eyes absent | EYA1 | x |
| | | | | homolog 1 | | |
| | | | | (Drosophila) | | |
| 109 | 3.95 | 0.038 | Down | immunoglobulin | IGLJ3 | |
| | | | | lambda joining 3 | | |
| 110 | −1.28 | 0.408 | None | — | — | |
| 111 | −1.19 | 0.543 | None | family with | FAM13A1 | |
| | | | | sequence similarity | | |
| | | | | 13, member A1 | | |
| 112 | −1.23 | 0.106 | None | chromosome 14 | C14orf18 | |
| | | | | open reading frame | | |
| | | | | 18 | | |
| 113 | 1.13 | 0.818 | None | H2A histone | H2AFY2 | |
| | | | | family, member Y2 | | |
| 68 | −1.63 | 0.261 | None | glucosaminyl (N- | GCNT3 | x |
| | | | | acetyl) transferase | | |
| | | | | 3, mucin type | | |
| 114 | −1.10 | 0.817 | None | heparan sulfate | HS3ST2 | |
| | | | | (glucosamine) 3-O- | | |
| | | | | sulfotransferase 2 | | |
| 115 | 1.25 | 0.371 | None | LBP protein; likely | LBP-9 | |
| | | | | ortholog of mouse | | |
| | | | | CRTR-1 | | |
| 116 | 1.45 | 0.650 | None | frizzled homolog | FZD10 | |
| | | | | 10 (Drosophila) | | |
| 117 | −1.49 | 0.063 | None | deafness locus | DELGEF | |
| | | | | associated putative | | |
| | | | | guanine nucleotide | | |
| | | | | exchange factor | | |
| 118 | −1.31 | 0.325 | None | aryl hydrocarbon | ARNTL2 | |
| | | | | receptor nuclear | | |
| | | | | translocator-like 2 | | |
| 119 | −1.92 | 0.182 | None | Homo sapiens | — | |
| | | | | transcribed | | |
| | | | | sequence with | | |
| | | | | weak similarity to | | |
| | | | | protein | | |
| | | | | ref: NP_009056.1 | | |
| | | | | (H. sapiens) | | |
| | | | | ubiquitously | | |
| | | | | transcribed | | |
| | | | | tetratricopeptide | | |
| | | | | repeat gene, Y | | |
| | | | | chromosome; | | |
| | | | | Ubiquitously | | |
| | | | | transcribed TPR | | |
| | | | | gene on Y | | |
| | | | | chromosome | | |
| | | | | [Homo sapiens] | | |
| 120 | −1.14 | 0.959 | None | CDW52 antigen | CDW52 | |
| | | | | (CAMPATH-1 | | |
| | | | | antigen) | | |

Key: \textcircled{1} Column
Description for Table 3
Table 3 lists all 55 genes which, after the evaluation of the raw data according to the statistical procedure from Example 4b, are relevant for a comparison of the gene expression between normal tissue (normal colon mucosa (Muc)), colorectal primary tumor (Tu) and liver metastasis (Lm) of these colorectal carcinomas.
Column 1 contains the SEQ ID NOS of the genes
Column 2 contains the Affymetrix Accession Numbers from the HG U-133-A Microarray
Column 3 contains the mean values of the gene expression of (n = 3) normal mucosa
Column 4 contains the mean values of the gene expression of (n = 10) colorectal primary tumors
Column 5 contains the mean values of the gene expression of (n = 20) liver metastases (corresponding with colorectal carcinoma)
Column 6 contains the difference in the gene expression between tumor and normal tissue indicated as fold-change (Fc)
Column 7 shows the significance level in the t-test
Column 8 shows whether the individual genes in the tumor are upregulated (up) or downregulated (down) or not significantly different (none) (in comparison to the normal tissue)
Column 9 contains the difference in the gene expression between liver metastases and tumor indicated as fold-change (Fc)
Column 10 shows the significance level in the t-test
Column 11 shows whether the individual genes of the liver metastasis have been upregulated (up) or downregulated (down) or are not significantly different (none) (in comparison to the tumor)
Column 12 contains the names of the genes
Column 13 contains the gene symbol
Column 14 marks the gene which was chosen both in Table 2 and in Table 3 with a “x”

TABLE 4
Expression data after statistical evaluation according to Example 4b of the responder (Resp)
versus non-responder (Non-Resp) to a 5-FU containing palliative chemotherapy

| \textcircled{1} | | | | | | | | |
|---|---|---|---|---|---|---|---|---|
| Spalte 1 | | | | Spalte 5 | | Spalte 7 | | |
| SEQ | | Spalte 3 | Spalte 4 | Fc_Resp | Spalte 6 | Direction | | |
| ID | Spalte 2 | Avg | Avg Non- | vs Non- | T- | Non-Resp | Spalte 8 | Spalte 9 |
| NO: | Affy Nr. | Responder | Responder | Resp | Test | vs Resp | Gene Name | Gene |
| 73 | 201564_s_at | 78.93 | 225.8 | −2.86 | 0.001 | Up | fascin homolog 1, actin-bundling protein | FSCN1 |
| | | | | | | | (Strongylocentrotus purpuratus) | |
| 74 | 201645_at | 309.55 | 737.18 | −2.38 | 0.011 | Up | tenascin C (hexabrachion) | TNC |
| 75 | 202973_x_at | 4587.02 | 2078.33 | 2.21 | 0.002 | Down | family with sequence similarity 13, member A1 | FAM13A1 |
| 76 | 203180_at | 385.6 | 1322 | −3.43 | 0.001 | Up | aldehyde dehydrogenase 1 family, member A3 | ALDH1A3 |
| 77 | 203649_s_at | 110.13 | 329.98 | −3 | 0.031 | Up | phospholipase A2, group IIA (platelets, synovial | PLA2G2A |
| | | | | | | | fluid) | |
| 78 | 203708_at | 154.7 | 944.08 | −6.1 | 0.003 | Up | phosphodiesterase 4B, cAMP-specific | PDE4B |
| | | | | | | | (phosphodiesterase E4 dunce homolog, | |
| | | | | | | | Drosophila) | |
| 79 | 203771_s_at | 73 | 152.42 | −2.09 | 0.002 | Up | biliverdin reductase A | BLVRA |
| 80 | 203819_s_at | 412.65 | 582.32 | −1.41 | 0.076 | Up | IGF-II mRNA-binding protein 3 | IMP-3 |
| 81 | 203837_at | 678.42 | 1604.02 | −2.36 | 0.016 | Up | mitogen-activated protein kinase kinase kinase 5 | MAP3K5 |
| 10 | 204073_s_at | 359.77 | 1108.27 | −3.08 | 0.029 | Up | chromosome 11 open reading frame 9 | C11orf9 |
| 14 | 204542_at | 119.03 | 432.17 | −3.63 | 0.014 | Up | sialyltransferase | STHM |
| 82 | 204673_at | 176.35 | 3241.4 | −18.38 | 0.123 | Up | mucin 2, intestinal/tracheal /// mucin 2, | MUC2 |
| | | | | | | | intestinal/tracheal | |
| 83 | 205097_at | 1437.68 | 165.6 | 8.68 | 0.011 | Down | solute carrier family 26 (sulfate transporter), | SLC26A2 |
| | | | | | | | member 2 | |
| 25 | 205240_at | 1539.52 | 435.67 | 3.53 | 0.005 | Down | LGN protein | LGN |
| 84 | 205278_at | 72.75 | 465.48 | −6.4 | 0.015 | Up | glutamate decarboxylase 1 (brain, 67 kDa) | GAD1 |
| 85 | 205372_at | 577.25 | 225.62 | 2.56 | 0.007 | Down | pleiomorphic adenoma gene 1 | PLAG1 |
| 86 | 205414_s_at | 909.3 | 198.15 | 4.59 | 0.004 | Down | KIAA0672 gene product | KIAA0672 |
| 87 | 205767_at | 950.72 | 440.02 | 2.16 | 0.024 | Down | epiregulin | EREG |
| 88 | 205865_at | 343.2 | 125.48 | 2.74 | 0.009 | Down | dead ringer-like 1 (Drosophila) | DRIL1 |
| 89 | 205927_s_at | 424.82 | 6797.2 | −16 | 0.037 | Up | cathepsin E | CTSE |
| 90 | 205937_at | 247.75 | 120.5 | 2.06 | 0.008 | Down | cell growth regulator with EF hand domain 1 | OGREF1 |
| 91 | 206884_s_at | 49.95 | 245.05 | −4.91 | 0.015 | Up | sciellin | SCEL |
| 92 | 207011_s_at | 290.3 | 97.75 | 2.97 | 0.001 | Down | PTK7 protein tyrosin kinase 7 | PTK7 |
| 93 | 207039_at | 199.7 | 575.47 | −2.88 | 0.017 | Up | cyclin-dependent kinase inhibitor 2A (melanoma, | CDKN2A |
| | | | | | | | p16, inhibits CDK4) | |
| 94 | 207063_at | 100.38 | 435.75 | −4.34 | 0.298 | Up | chromosome Y open reading frame 14 | CYorf14 |
| 95 | 207509_s_at | 111.95 | 267.15 | −2.39 | 0.008 | Up | leukocyte-associated Ig-like receptor 2 | LAIR2 |
| 96 | 208262_x_at | 88.7 | 25.38 | 3.49 | 0.046 | Down | Mediterranean fever | MEFV |
| 41 | 208510_s_at | 3005.18 | 776.35 | 3.87 | 0.023 | Down | peroxisome proliferative activated receptor, | PPARG |
| | | | | | | | gamma | |
| 97 | 209125_at | 85.85 | 389.77 | −4.54 | 0.028 | Up | keratin 6A | KRT6A |
| 98 | 209436_at | 430.63 | 2883 | −6.69 | 0.003 | Up | spondin 1, (f-spondin) extracellular matrix | SPON1 |
| | | | | | | | protein | |
| 99 | 209905_at | 7293.25 | 2925.58 | 2.49 | 0.040 | Down | homeo box A9 | HOXA9 |
| 100 | 210164_at | 1260.1 | 1018.28 | 1.24 | 0.004 | Down | granzyme B (granzyme 2, cytotoxic T- | GZMB |
| | | | | | | | lymphocyte-associated serine esterase 1) | |
| 101 | 211372_s_at | 170.82 | 520.53 | −3.05 | 0.004 | Up | interleukin 1 receptor, type II | IL1R2 |
| 102 | 211430_s_at | 247.55 | 1745.92 | −7.05 | 0.027 | Up | immunoglobulin heavy constant gamma 3 (G3m | IGHG3 |
| | | | | | | | marker) | |
| 103 | 212349_at | 1049.9 | 617.35 | 1.7 | 0.001 | Down | protein O-fucosyltransferase 1 | POFUT1 |
| 104 | 212531_at | 1850.23 | 9150.78 | −4.95 | 0.053 | Up | lipocalin 2 (oncogene 24p3) | LCN2 |
| 52 | 212850_s_at | 3434.2 | 1111.52 | 3.09 | 0.043 | Down | low density lipoprotein receptor-related protein 4 | LRP4 |
| 105 | 213230_at | 224.27 | 529.05 | −2.36 | 0.001 | Up | paraneoplastic antigen | HUMPPA |
| 106 | 213435_at | 3523.13 | 2242.65 | 1.57 | 0.001 | Down | SATB family member 2 | SATB2 |
| 107 | 213880_at | 2524 | 606.27 | 4.16 | | Down | G protein-coupled receptor 49 | GPR49 |
| 108 | 213994_s_at | 73.4 | 967.63 | −13.18 | 0.001 | Up | spondin 1, (f-spondin) extracellular matrix | SPON1 |
| | | | | | | | protein | |
| 63 | 214608_s_at | 572.88 | 28.28 | 20.26 | 0.014 | Down | eyes absent homolog 1 (Drosophila) | EYA1 |
| 109 | 214677_x_at | 268.77 | 1507.35 | −5.61 | 0.144 | Up | immunoglobulin lambda joining 3 | IGLJ3 |
| 110 | 216910_at | 112.45 | 43.92 | 2.56 | 0.098 | Down | — | — |
| 111 | 217047_s_at | 3108.7 | 1366.12 | 2.28 | 0.002 | Down | family with sequence similarity 13, member A1 | FAM13A1 |
| 112 | 217988_at | 4618.63 | 2386.77 | 1.94 | 0.001 | Down | chromosome 14 open reading frame 18 | C14orf18 |
| 113 | 218445_at | 777.8 | 197.75 | 3.93 | 0.014 | Down | H2A histone family, member Y2 | H2AFY2 |
| 68 | 219508_at | 2260.57 | 9751.88 | −4.31 | 0.046 | Up | glucosaminyl (N-acetyl) transferase 3, mucin | GCNT3 |
| | | | | | | | type | |
| 114 | 219697_at | 205.07 | 350.1 | −1.71 | 0.005 | Up | heparan sulfate (glucosamine) 3-O- | HS3ST2 |
| | | | | | | | sulfotransferase 2 | |
| 115 | 219735_s_at | 1344.83 | 611.62 | 2.2 | 0.002 | Down | LBP protein; likely ortholog of mouse CRTR-1 | LBP-9 |
| 116 | 219764_at | 1375.78 | 76.05 | 18.09 | 0.073 | Down | frizzled homolog 10 (Drosophila) | FZD10 |
| 117 | 220482_s_at | 314.05 | 107.65 | 2.92 | 0.001 | Down | deafness locus associated putative guanine | DELGEF |
| | | | | | | | nucleotide exchange factor | |
| 118 | 220658_s_at | 181.05 | 302.38 | −1.67 | 0.003 | Up | aryl hydrocarbon receptor nuclear translocator- | ARNTL2 |
| | | | | | | | like 2 | |
| 119 | 222347_at | 150.07 | 378.72 | −2.52 | 0.111 | Up | Homo sapiens transcribed sequence with week | — |
| | | | | | | | similarity to protein ref: NP_000056.1 | |
| | | | | | | | (H. sapiens) ubiquitously transcribed | |
| | | | | | | | tetratricopeptide repeat gene, Y chromosome; | |
| | | | | | | | Ubiquitously transcribed TPR gene on Y | |
| | | | | | | | chromosome [Homo sapiens] | |
| 120 | 34210_at | 91.93 | 1517.25 | −16.5 | 0.004 | Up | CDW52 antigen (CAMPATH-1 antigen) | CDW52 |

Key: \textcircled{1} Column
Description for Table 4
Table 4 lists all 55 genes which, after the evaluation of the raw data according to the statistical procedure from Example 4a, are relevant for a comparison of the gene expression of responders (Resp) with that of non-responders (Non-Resp) in reference to a response to a 5-FU-containing palliative chemotherapy in metastasized colorectal carcinoma.
Column 1 contains the SEQ ID NOS of the gene
Column 2 contains the Affymetrix Accession Numbers from the HG U-133-A Microarray
Column 3 contains the mean values of the gene expression of (n = 8) responders
Column 4 contains the mean values of the gene expression of (n = 9) non-responders
Column 5 contains the difference in the gene expression between responders and non-responders, indicated as fold-change (Fc)
Column 6 shows the significance level in the t-test
Column 7 shows whether the individual genes in the non-responders have been upregulated (up) or downregulated (down) (in comparison to the responders)
Column 8 contains the names of the genes
Column 9 contains the gene symbol

TABLE 5
Expression data of responders (Resp), non-responders (nonresp) and patient T of Example 16

| | | | | | Spalte 6 | |
|---|---|---|---|---|---|---|
| | | | | | \textcircled{2} Veränderung | Spalte 7 |
| \textcircled{1} Spalte 1 | | | | | des Expressions- | \textcircled{3} |
| SEQ ID | Spalte 2 | Spalte 3 | Spalte 4 | Spalte 5 | niveaus bci | Veranderung |
| NO: | Affy Nr | Pat. T | avg_resp | avg_nonresp | Respondern | bei Pat. T. |
| 1 | 200954_at | 133.7 | 145.65 | 519.68 | ↓ | ↓ |
| 2 | 201137_s_at | 674.6 | 1293.3 | 5177.28 | ↓ | ↓ |
| 3 | 201842_s_at | 691 | 871.47 | 3299.13 | ↓ | ↓ |
| 4 | 202953_at | 154.6 | 305.67 | 1092.85 | ↓ | ↓ |
| 5 | 202990_at | 823.1 | 174.3 | 768.48 | ↓ | ↑ |
| 6 | 203382_s_at | 20 | 132.48 | 513.97 | ↓ | ↓ |
| 7 | 203645_s_at | 238.8 | 353.65 | 1385.95 | ↓ | ↓ |
| 8 | 203895_at | 9887 | 12464.9 | 2658.78 | ↑ | ↑ |
| 9 | 203939_at | 1419.5 | 448.4 | 1686.73 | ↓ | ↓ |
| 10 | 204073_s_at | 143.9 | 359.77 | 1108.27 | ↓ | ↓ |
| 11 | 204143_s_at | 4459.1 | 469.02 | 1854.93 | ↓ | ↑ |
| 12 | 204204_at | 399.9 | 148.08 | 488.2 | ↓ | ↓ |
| 13 | 204416_x_at | 399.5 | 856.1 | 3347.48 | ↓ | ↓ |
| 14 | 204542_at | 33.7 | 119.03 | 432.17 | ↓ | ↓ |
| 15 | 204584_at | 37.7 | 389.25 | 70.62 | ↑ | ↓ |
| 16 | 204698_at | 13.5 | 181.85 | 590.4 | ↓ | ↓ |
| 17 | 204705_x_at | 149.2 | 270.88 | 1495.58 | ↓ | ↓ |
| 18 | 204714_s_at | 1567.1 | 231.23 | 1197.85 | ↓ | ↑ |
| 19 | 204965_at | 167.5 | 359.55 | 1387.28 | ↓ | ↓ |
| 20 | 204988_at | 1741.6 | 2708 | 13973.72 | ↓ | ↓ |
| 21 | 204992_s_at | 3713.2 | 4082.15 | 905.9 | ↑ | ↑ |
| 22 | 205041_s_at | 99.7 | 196.2 | 2513.82 | ↓ | ↓ |
| 23 | 205108_s_at | 68.8 | 209.98 | 845.3 | ↓ | ↓ |
| 24 | 205229_s_at | 1444.8 | 804.8 | 209.83 | ↑ | ↑ |
| 25 | 205240_at | 1930.1 | 1539.52 | 435.67 | ↑ | ↑ |
| 26 | 205477_s_at | 75.7 | 283.2 | 1155.3 | ↓ | ↓ |
| 27 | 205522_at | 348.5 | 726.27 | 133.53 | ↑ | ↑ |
| 28 | 205604_at | 468.6 | 462.38 | 102.25 | ↑ | ↑ |
| 29 | 205650_s_at | 291.3 | 493.55 | 2904.28 | ↓ | ↓ |
| 30 | 205754_at | 57.9 | 209.75 | 662.38 | ↓ | ↓ |
| 31 | 205755_at | 89 | 141.5 | 680.27 | ↓ | ↓ |
| 32 | 205923_at | 341.9 | 227.28 | 807.67 | ↓ | ↓ |
| 33 | 205969_at | 432.1 | 175.48 | 757.43 | ↓ | ↓ |
| 34 | 206042_x_at | 14.4 | 896.6 | 212.67 | ↑ | ↓ |
| 35 | 206130_s_at | 90.5 | 124.9 | 477.2 | ↓ | ↓ |
| 36 | 206287_s_at | 16.7 | 24.95 | 372.38 | ↓ | ↓ |
| 37 | 206350_at | 430.1 | 555.53 | 2352.27 | ↓ | ↓ |
| 38 | 207267_s_at | 320.6 | 575.88 | 164.03 | ↑ | ↑ |
| 39 | 207761_s_at | 1483.8 | 1638.63 | 5869 | ↓ | ↓ |
| 40 | 208470_s_at | 42.2 | 1226.9 | 15875.23 | ↓ | ↓ |
| 41 | 208510_s_at | 2092.3 | 3005.18 | 776.35 | ↑ | ↑ |
| 42 | 208962_s_at | 894.8 | 1947.03 | 556.83 | ↑ | ↑ |
| 43 | 209555_s_at | 106.7 | 74.35 | 315.28 | ↓ | ↓ |
| 44 | 209699_x_at | 1552.7 | 709.47 | 3046.28 | ↓ | ↓ |
| 45 | 209901_x_at | 20.1 | 46.32 | 346.83 | ↓ | ↓ |
| 46 | 210619_s_at | 18.3 | 37.33 | 301.33 | ↓ | ↓ |
| 47 | 211367_s_at | 94.8 | 88.02 | 393.8 | ↓ | ↓ |
| 48 | 211429_s_at | 1051.3 | 3552.52 | 19699.27 | ↓ | ↓ |
| 49 | 211991_s_at | 130.3 | 229.8 | 1334.97 | ↓ | ↓ |
| 50 | 212224_at | 1499.7 | 141.23 | 716.03 | ↓ | ↑ |
| 51 | 212235_at | 39.5 | 135.98 | 575.18 | ↓ | ↓ |
| 52 | 212850_s_at | 5279 | 3434.2 | 1111.52 | ↑ | ↑ |
| 53 | 213204_at | 247 | 192.18 | 930.65 | ↓ | ↓ |
| 54 | 213343_s_at | 339.1 | 419.13 | 119.17 | ↑ | ↑ |
| 55 | 213568_at | 119.1 | 145.08 | 454.45 | ↓ | ↓ |
| 56 | 213592_at | 315 | 329.2 | 1239.5 | ↓ | ↓ |
| 57 | 213988_s_at | 2517.6 | 1016.7 | 3291.8 | ↓ | ↓ |
| 58 | 214063_s_at | 64.4 | 237.9 | 1677.93 | ↓ | ↓ |
| 59 | 214428_x_at | 632.5 | 779.75 | 2975.48 | ↓ | ↓ |
| 60 | 214456_x_at | 43.1 | 264.88 | 4909.38 | ↓ | ↓ |
| 61 | 214465_at | 30.7 | 66.25 | 762.43 | ↓ | ↓ |
| 62 | 214604_at | 671 | 1215.43 | 131.85 | ↑ | ↑ |
| 63 | 214608_s_at | 56.4 | 572.88 | 28.28 | ↑ | ↑ |
| 64 | 214669_x_at | 43.4 | 200.98 | 624.15 | ↓ | ↓ |
| 65 | 215388_s_at | 180.5 | 336.75 | 1303.15 | ↓ | ↓ |
| 66 | 217767_at | 545.6 | 953.25 | 5588.37 | ↓ | ↓ |
| 67 | 218232_at | 103.5 | 93.93 | 390.22 | ↓ | ↓ |
| 68 | 219508_at | 1915.7 | 2260.57 | 9751.88 | ↓ | ↓ |
| 69 | 219612_s_at | 785.1 | 1133.45 | 7741.42 | ↓ | ↓ |
| 70 | 222162_s_at | 82.5 | 183.68 | 646.53 | ↓ | ↓ |
| 71 | 37020_at | 264.3 | 462.3 | 3024.4 | ↓ | ↓ |
| 72 | 39763_at | 132.1 | 185.4 | 988.95 | ↓ | ↓ |
| 73 | 201564_s_at | 66.3 | 78.93 | 225.8 | ↓ | ↓ |
| 74 | 201645_at | 92.9 | 309.55 | 737.18 | ↓ | ↓ |
| 75 | 202973_x_at | 10022.1 | 4587.02 | 2078.33 | ↑ | ↑ |
| 76 | 203180_at | 35 | 385.6 | 1322 | ↓ | ↓ |
| 77 | 203649_s_at | 264.2 | 110.13 | 329.98 | ↓ | ↓ |
| 78 | 203708_at | 72.6 | 154.7 | 944.08 | ↓ | ↓ |
| 79 | 203771_s_at | 8.6 | 73 | 152.42 | ↓ | ↓ |
| 80 | 203819_s_at | 378.7 | 412.65 | 582.32 | ↓ | ↓ |
| 81 | 203837_at | 1664.9 | 678.42 | 1604.02 | ↓ | ↑ |
| 82 | 204673_at | 40.4 | 176.35 | 3241.4 | ↓ | ↓ |
| 83 | 205097_at | 2962.1 | 1437.68 | 165.6 | ↑ | ↑ |
| 84 | 205278_at | 13.9 | 72.75 | 465.48 | ↓ | ↓ |
| 85 | 205372_at | 2399.6 | 577.25 | 225.62 | ↑ | ↑ |
| 86 | 205414_s_at | 43.9 | 909.3 | 198.15 | ↑ | ↓ |
| 87 | 205767_at | 1720.8 | 950.72 | 440.02 | ↑ | ↑ |
| 88 | 205865_at | 402.3 | 343.2 | 125.48 | ↑ | ↑ |
| 89 | 205927_s_at | 68.5 | 424.82 | 6797.2 | ↓ | ↓ |
| 90 | 205937_at | 218.7 | 247.75 | 120.5 | ↑ | ↑ |
| 91 | 206884_s_at | 97.9 | 49.95 | 245.05 | ↓ | ↓ |
| 92 | 207011_s_at | 178.4 | 290.3 | 97.75 | ↑ | ↑ |
| 93 | 207039_at | 342 | 199.7 | 575.47 | ↓ | ↓ |
| 94 | 207063_at | 62.9 | 100.38 | 435.75 | ↓ | ↓ |
| 95 | 207509_s_at | 63 | 111.95 | 267.15 | ↓ | ↓ |
| 96 | 208262_x_at | 101.3 | 88.7 | 25.38 | ↑ | ↑ |
| 97 | 209125_at | 45.7 | 85.85 | 389.77 | ↓ | ↓ |
| 98 | 209436_at | 98.8 | 430.63 | 2883 | ↓ | ↓ |
| 99 | 209905_at | 6248.2 | 7293.25 | 2925.58 | ↑ | ↑ |
| 100 | 210164_at | 824.8 | 1260.1 | 1018.28 | ↑ | ↓ |
| 101 | 211372_s_at | 34.5 | 170.82 | 520.53 | ↓ | ↓ |
| 102 | 211430_s_at | 112.9 | 247.55 | 1745.92 | ↓ | ↓ |
| 103 | 212349_at | 1199.6 | 1049.9 | 617.35 | ↑ | ↑ |
| 104 | 212531_at | 261.7 | 1850.23 | 9150.78 | ↓ | ↓ |
| 105 | 213230_at | 174.7 | 224.27 | 529.05 | ↓ | ↓ |
| 106 | 213435_at | 7949.7 | 3523.13 | 2242.65 | ↑ | ↑ |
| 107 | 213880_at | 6388.3 | 2524 | 606.27 | ↑ | ↑ |
| 108 | 213994_s_at | 44.1 | 73.4 | 967.63 | ↓ | ↓ |
| 109 | 214677_x_at | 26.7 | 268.77 | 1507.35 | ↓ | ↓ |
| 110 | 216910_at | 56.6 | 112.45 | 43.92 | ↑ | ↑ |
| 111 | 217047_s_at | 5878.7 | 3108.7 | 1366.12 | ↑ | ↑ |
| 112 | 217988_at | 3909.6 | 4618.63 | 2386.77 | ↑ | ↑ |
| 113 | 218445_at | 515.3 | 777.8 | 197.75 | ↑ | ↑ |
| 114 | 219697_at | 122.7 | 205.07 | 350.1 | ↓ | ↓ |
| 115 | 219735_s_at | 559.9 | 1344.83 | 611.62 | ↑ | ↓ |
| 116 | 219764_at | 45.7 | 1375.78 | 76.05 | ↑ | ↓ |
| 117 | 220482_s_at | 285.3 | 314.05 | 107.65 | ↑ | ↑ |
| 118 | 220658_s_at | 235.3 | 181.05 | 302.38 | ↓ | ↓ |
| 119 | 222347_at | 325.8 | 150.07 | 378.72 | ↓ | ↓ |
| 120 | 34210_at | 6.9 | 91.93 | 1517.25 | ↓ | ↓ |

Key:
\textcircled{1} Column
\textcircled{2} Change in the expression level of responders
\textcircled{3} Change in patient T.
Description for Table 5
Table 5 compares the change of the expression level between patient T., responders (Resp) and non-responders (Non-Resp).
Column 1 contains the SEQ ID NOS of the gene
Column 2 contains the Affymetrix Accession Numbers on the HG U-133-A Microarray
Column 3 contains the gene expression of in the liver metastasis of patient T.
Column 4 contains the mean values of the gene expression in the liver metastasis of responders (Resp)
Column 5 contains the mean values of the gene expression in the liver metastasis of non-responders (non-Resp)
Column 6 shows the average change of the expression level in responders
Column 7 shows the change of the expression level in patient T.

One sees a good agreement between columns 6 and 7 (changes in the expression level that do not match are printed in bold). In total, 66 of the 72 genes from Table 1 (91.7%) and 50 of 55 genes from Table 4 (90.9%) were correctly assigned, which means that patient T., in reference to the gene signature, belongs with high probability to the responders.

In particular, 27 of the 28 genes of the “minimal set” agree (only one deviation at number 116). Thus the probability that patient T. is a responder is 96%.

(The expression data of patient T. were not included in the computations, which are based on columns 4 and 5.)

TABLE 6
Expression data colon/rectum mucosa (Muc) versus colorectal primary tumor (Tu) of
n = 12 independent patients according to Example 17 as validation of Table 2

| | | | | Spalte 5 | Spalte 6 | Spalte 7 | | |
|---|---|---|---|---|---|---|---|---|
| \textcircled{1} Spalte 1 | | Spalte 3 | | Fc_Tu | T-Test | Direction | Spalte 8 | Spalte 9 |
| Seq | Spalte 2 | Avg | Spalte 4 | vs | Tu vs | Tu vs | \textcircled{2} | \textcircled{3} |
| ID | Affy_Nr | Mucosa | Avg Tu | Muc | Muc | Muc | Gen-Name | Gen |
| 25 | 205240_at | 305.42 | 2612.66 | 8.55 | 0.001 | Up | LGN protein | LGN |
| 41 | 208510_s_at | 712.09 | 410.26 | −1.74 | 0.002 | Down | Peroxisome | PPARG |
| | | | | | | | proliferative | |
| | | | | | | | activated | |
| | | | | | | | receptor, | |
| | | | | | | | gamma | |
| 68 | 219508_at | 14667.20 | 5657.67 | −2.59 | 0.001 | Down | Glucosaminyl | GCNT3 |
| | | | | | | | (N-acetyl) | |
| | | | | | | | transferase | |
| | | | | | | | 3. mucin type | |

Key:
\textcircled{1} Column
\textcircled{2} Gene name
\textcircled{3} Gene
Description for Table 6
Table 6 lists the Seq ID which, according to Claim 9, allow a differentiation between normal intestinal mucosa and colorectal carcinoma.
Column 1 contains the SEQ ID NOS of the gene
Column 2 contains the Affymetrix Accession Numbers from the HG U-133-A Microarray
Column 3 contains the mean values of the gene expression of (n = 12) colon/rectum mucosa biopsies
Column 4 contains the mean values of the gene expression of (n = 12; corresponding to column 3) colon/rectal carcinoma biopsies
Column 5 contains the difference in the gene expression between tumor and normal tissue indicated as fold-change (Fc)
Column 6 shows the significance level in the t-test
Column 7 shows whether the individual genes in the tumor are upregulated (up) or downregulated (down) (in comparison to normal tissue)
Column 8 contains the names of the genes
Column 9 contains the gene symbol

TABLE A
Prediction of the response of liver metastases to a 5-FU-containing chemotherapy by comparing
responders (Resp; n = 10) and non-responders (Non-Resp; n = 10).

| | | | | | Fold | | | |
|---|---|---|---|---|---|---|---|---|
| | | | | \textcircled{2} | change | \textcircled{3} | | |
| \textcircled{1} Auswahl aus | | | | Expressionsniveau | (Resp | Statistische Signifikanz | | |
| \textcircled{4} 72 Genen | \textcircled{5} 55 Genen | Minimal set | | (Resp vs Non- | vs Non- | (Resp vs Non-Resp) | | |
| (Tab. 1) | (Tab. 4) | \textcircled{6} Spalte 3 | Spalte 4 | Resp) | Resp) | T-Test | Welch | Wllcoxon |
| | | 6 | 6 | ↑ | (−3)-(−5) | 0.000692 | 0.001295 | 0.001243 |
| \textcircled{7} alle | alle | 7 | 7 | ↑ | (−3)-(−5) | 0.002463 | 0.002208 | 0.002176 |
| 72 | 55 | 8 | 8 | ↓ | 4-6 | 0.000500 | 0.004928 | 0.000311 |
| \textcircled{8} Gene | Gene | 10 | | ↑ | (−2)-(−4) | 0.001225 | 0.001537 | 0.001243 |
| | | 13 | 13 | ↑ | (−3)-(−5) | 0.000818 | 0.000815 | 0.003730 |
| | | 14 | | ↑ | (−2)-(−5) | 0.002650 | 0.003274 | 0.001243 |
| | | 24 | 24 | ↓ | 3-5 | 0.000170 | 0.000549 | 0.001243 |
| | | 25 | | ↓ | 2-5 | 0.003655 | 0.008318 | 0.005905 |
| | | 41 | | ↓ | 3-5 | 0.001252 | 0.001718 | 0.003730 |
| | | 45 | 45 | ↑ | (−6)-(−9) | 0.004320 | 0.009322 | 0.009324 |
| | | 46 | 46 | ↑ | (−6)-(−10) | 0.000412 | 0.001005 | 0.006488 |
| | | 48 | 48 | ↑ | (−4)-(−7) | 0.001342 | 0.001544 | 0.001243 |
| | | 52 | 52 | ↓ | 2-4 | 0.007332 | 0.017840 | 0.054080 |
| | | 54 | 54 | ↓ | 2-5 | 0.008371 | 0.012090 | 0.013990 |
| | | 60 | 60 | ↑ | (−15)-(−21) | 0.000174 | 0.000388 | 0.001243 |
| | | 61 | 61 | ↑ | (−9)-(−13) | 0.000107 | 0.000480 | 0.001243 |
| | | 63 | | ↓ | 16-24 | 0.096520 | 0.090670 | 0.147600 |
| | | 65 | 65 | ↑ | (−3)-(−5) | 0.003568 | 0.003485 | 0.003730 |
| | | 66 | 66 | ↑ | (−5)-(−7) | 0.002488 | 0.002510 | 0.002176 |
| | | 68 | | ↑ | (−3)-(−5) | 0.000268 | 0.000282 | 0.000311 |
| | | 73 | 73 | ↑ | (−2)-(−4) | 0.000016 | 0.000015 | 0.000311 |
| | | 78 | 78 | ↑ | (−5)-(−7) | 0.002634 | 0.004401 | 0.005905 |
| | | 79 | 79 | ↑ | (−1)-(−3) | 0.002341 | 0.002162 | 0.003730 |
| | | 80 | 80 | ↑ | (−1)-(−3) | 0.076230 | 0.073200 | 0.152000 |
| | | 89 | 89 | ↑ | (−13)-(−19) | 0.037240 | 0.056650 | 0.093860 |
| | | 97 | 97 | ↑ | (−4)-(−6) | 0.028840 | 0.054100 | 0.028900 |
| | | 98 | 98 | ↑ | (−6)-(−8) | 0.003484 | 0.009664 | 0.009324 |
| | | 101 | 101 | ↑ | (−2)-(−4) | 0.004217 | 0.005559 | 0.009324 |
| | | 104 | 104 | ↑ | (−4)-(−6) | 0.005314 | 0.009279 | 0.003730 |
| | | 107 | 107 | ↓ | 3-5 | 0.001421 | 0.002313 | 0.005905 |
| | | 108 | 108 | ↑ | (−11)-(−15) | 0.001185 | 0.001994 | 0.002176 |
| | | 116 | 116 | ↓ | 15-21 | 0.073320 | 0.069390 | 0.231900 |
| | | 117 | 117 | ↓ | 2-4 | 0.000512 | 0.003814 | 0.000311 |
| | | 120 | 120 | ↑ | (−13)-(−19) | 0.004197 | 0.006615 | 0.005905 |
| 72 | 55 | 34 | 28 | 34 | | | | |

Key:
\textcircled{1} Selection of
\textcircled{2} Expression level
\textcircled{3} Statistical significance (Resp vs Non-Resp)
\textcircled{4} 72 genes (Table 1)
\textcircled{5} 55 genes (Table 4)
\textcircled{6} Column
\textcircled{7} All
\textcircled{8} Genes

TABLE B
Early detection of a colorectal carcinoma by comparing healthy
mucosa (Muc; n = 3) and colorectal carcinoma (Tu; n = 10)

| \textcircled{1} Auswahl aus | | | | |
|---|---|---|---|---|
| \textcircled{3} 72 | \textcircled{4} 55 | | | |
| Genen | Genen | Minimal | \textcircled{2} Δ Expressionsniveau | Fold change |
| (Tab. 2) | (Tab. 3) | set | (Muc vs Tu) | (Muc vs Tu) |
| 2 | | | ↓ | 4-6 |
| 7 | | | ↓ | 1-3 |
| 8 | | | ↑ | (−4)-(−2) |
| 12 | | | ↓ | 1-3 |
| 19 | | | ↓ | 1-3 |
| 21 | | | ↑ | (−1)-(−3) |
| 25 | 25 | 25 | ↑ | (−3)-(−5) |
| 33 | | | ↑ | (−4)-(−6) |
| 38 | | | ↑ | (−4)-(−6) |
| 41 | 41 | 41 | ↓ | 1-3 |
| 45 | | | ↓ | 2-4 |
| 49 | | | ↓ | 6-8 |
| 50 | | | ↓ | 2-4 |
| 51 | | | ↑ | (−3)-(−5) |
| 54 | | | ↑ | (−5)-(−7) |
| 66 | | | ↓ | 1-3 |
| 68 | 68 | 68 | ↓ | 3-5 |
| 70 | | | ↓ | 3-5 |
| | 78 | | ↓ | 1-3 |
| | 83 | | ↓ | 10-14 |
| | 85 | | ↑ | (−2)-(−4) |
| | 86 | | ↓ | 3-5 |
| | 92 | | ↑ | (−2)-(−4) |
| | 99 | | ↑ | (−2)-(−4) |
| | 101 | | ↓ | 6-10 |
| | 103 | | ↑ | (−2)-(−4) |
| | 104 | | ↑ | (−12)-(−17) |
| | 105 | | ↑ | (−4)-(−6) |
| | 109 | | ↓ | 6-8 |
| | 112 | | ↑ | (−1)-(−3) |
| | 114 | | ↓ | 1-3 |
| | 115 | | ↓ | 1-3 |
| | 118 | | ↑ | (−2)-(−4) |
| 18 | 18 | 3 | 33 | |

Key:
\textcircled{1} Selection from
\textcircled{2} Δ Expression level (Muc vs Tu)
\textcircled{3} 72 genes (Table 2)
\textcircled{4} 55 genes (Table 3)

TABLE C
Prediction of liver metastases by comparing liver metastases
(Lm; n = 20) and colorectal carcinoma (Tu; n = 10)

\textcircled{1} Auswahl aus

| \textcircled{3} 72 | \textcircled{4} 55 | | | |
|---|---|---|---|---|
| Genen | Genen | Minimal | \textcircled{2} Δ Expressionsniveau | Fold change |
| (Tab. 2) | (Tab. 3) | set | (Lm vs Tu) | (Lm vs Tu) |
| 17 | | 17 | ↑ | (−2)-(−4) |
| 19 | | 19 | ↑ | (−12)-(−18) |
| 20 | | 20 | ↑ | (−69)-(−99) |
| 22 | | 22 | ↑ | (−26)-(−34) |
| 26 | | 26 | ↑ | (−18)-(−22) |
| 29 | | 29 | ↑ | (−10)-(−16) |
| 30 | | 30 | ↑ | (−6)-(−9) |
| 31 | | 31 | ↑ | (−9)-(−13) |
| 33 | | 33 | ↑ | (−3)-(−5) |
| 35 | | 35 | ↑ | (−4)-(−6) |
| 37 | | 37 | ↑ | (−6)-(−10) |
| 40 | | 40 | ↑ | (−82)-(−124) |
| 48 | | 48 | ↑ | (−3)-(−5) |
| 58 | | 58 | ↑ | (−13)-(−19) |
| 59 | | 59 | ↑ | (−2)-(−4) |
| 64 | | 64 | ↓ | 2-4 |
| 66 | | 66 | ↑ | (−4)-(−6) |
| 69 | | 69 | ↑ | (−66)-(−96) |
| 71 | | 71 | ↑ | (−69)-(−99) |
| 72 | | 72 | ↑ | (−5)-(−7) |
| | 74 | 74 | ↓ | 1-3 |
| | 109 | 109 | ↓ | 3-5 |
| 20 | 2 | 22 | 22 | |

Key:
\textcircled{1} Selection from
\textcircled{2} Δ Expression level (Muc vs Tu)
\textcircled{3} 72 genes (Table 2)
\textcircled{4} 55 genes (Table 3)

TABLE D
Congruence list of the assigned SEQ ID NOs

| | | \textcircled{2} Varianten | | |
|---|---|---|---|---|
| | SEQ ID | dargestellt in | | |
| \textcircled{1} Genname | NO | SEQ ID NO | Affy Nr | Gene |
| ATPase, H+ transporting, lysosomal | 1 | | 200954_at | ATP6V0C |
| 16 kDa, V0 subunit c | | | | |
| major histocompatibility complex, | 2 | | 201137_s_at | HLA-DPB1 |
| class II, DP beta 1 | | | | |
| EGF-containing fibulin-like | 3 | 121 | 201842_s_at | EFEMP1 |
| extracellular matrix protein 1 | | | | |
| complement component 1, q | 4 | | 202953_at | C1QB |
| subcomponent, beta polypeptide | | | | |
| phosphorylase, glycogen; liver (Hers | 5 | | 202990_at | PYGL |
| disease) | | | | |
| apolipoprotein E | 6 | | 203382_s_at | APOE |
| CD163 antigen | 7 | | 203645_s_at | CD163 |
| phospholipase C, beta 4 | 8 | 122 | 203895_at | PLCB4 |
| 5′-nucleotidase, ecto (CD73) | 9 | | 203939_at | NT5E |
| chromosome 11 open reading frame 9 | 10 | | 204073_s_at | C11orf9 |
| rTS beta protein | 11 | | 204143_s_at | HSRTSBETA |
| solute carrier family 31 (copper | 12 | | 204204_at | SLC31A2 |
| transporters), member 2 | | | | |
| apolipoprotein C-I | 13 | | 204416_x_at | APOC1 |
| sialyltransferase | 14 | | 204542_at | STHM |
| L1 cell adhesion molecule | 15 | 123 | 204584_at | LICAM |
| (hydrocephalus, stenosis of aqueduct | | | | |
| of Sylvius 1) | | | | |
| interferon stimulated gene 20 kDa | 16 | | 204698_at | ISG20 |
| aldolase B, fructose-bisphosphate | 17 | | 204705_x_at | ALDOB |
| coagulation factor V (proaccelerin, | 18 | | 204714_s_at | F5 |
| labile factor) | | | | |
| group-specific component (vitamin D | 19 | | 204965_at | GC |
| binding protein) | | | | |
| fibrinogen, B beta polypeptide | 20 | | 204988_at | FGB |
| profilin 2 | 21 | 124 | 204992_s_at | PFN2 |
| orosomucoid 1 | 22 | | 205041_s_at | ORM1 |
| apolipoprotein B (including Ag(x) | 23 | | 205108_s_at | APOB |
| antigen) | | | | |
| coagulation factor C homolog, cochlin | 24 | | 205229_s_at | COCH |
| (Limulus polyphemus) | | | | |
| LGN protein | 25 | | 205240_at | LGN |
| alpha-1-microglobulin/bikunin | 26 | | 205477_s_at | AMBP |
| precursor | | | | |
| homeo box D4 | 27 | | 205522_at | HOXD4 |
| homeo box D9 | 28 | | 205604_at | HOXD9 |
| fibrinogen, A alpha polypeptide | 29 | 125 | 205650_s_at | FGA |
| coagulation factor II (thrombin) | 30 | | 205754_at | F2 |
| pre-alpha (globulin) inhibiter, H3 | 31 | | 205755_at | ITIH3 |
| polypeptide | | | | |
| reelin | 32 | 126 | 205923_at | RELN |
| arylacetamide deacetylase (esterase) | 33 | | 205969_at | AADAC |
| small nuclear ribonucleoprotein | 34 | 127, 128, 129, | 206042_x_at | SNRPN |
| polypeptide N | | 130, 131, 132 | | |
| asialoglycoprotein receptor 2 | 35 | 133, 134, 135 | 206130_s_at | ASGR2 |
| inter-alpha (globulin) inhibitor H4 | 36 | | 206287_s_at | ITIH4 |
| (plasma Kallikrein-sensitive | | | | |
| glycoprotein) | | | | |
| amyloid P component, serum | 37 | | 206350_at | APCS |
| Down syndrome critical region gene 6 | 38 | | 207267_s_at | DSCR6 |
| DKFZP586A0522 protein | 39 | | 207761_s_at | DKFZP586A0522 |
| haptoglobin | 40 | | 208470_s_at | HP |
| peroxisome proliferative activated | 41 | 136, 137, 138 | 208510_s_at | PPARG |
| receptor, gamma | | | | |
| fatty acid desaturase 1 | 42 | | 208962_s_at | FADS1 |
| CD36 antigen (collagen type I | 43 | | 209555_s_at | CD36 |
| receptor, thrombospondin receptor) | | | | |
| aldo-keto reductase family 1, member | 44 | | 209699_x_at | AKR1C2 |
| C2 (dihydrodiol dehydrogenase 2) | | | | |
| allograft inflammatory factor 1 | 45 | 139, 140 | 209901_x_at | AIF1 |
| hyaluronoglucosaminidase 1 | 46 | 141, 142, 143, | 210619_s_at | HYAL1 |
| | | 144, 145, 146, | | |
| | | 147 | | |
| caspase 1, apoptosis-related cysteine | 47 | 148, 149, 150 | 211367_s_at | CASP1 |
| protease (ICE-1) | | | | |
| alpha-1 Antitrypsin | 48 | 151, 152 | 211429_s_at | SERPINA1 |
| major histocompatibility complex, | 49 | | 211991_s_at | FILA-DPA1 |
| class II, DP alpha 1 | | | | |
| aldehyde dehydrogenase 1 family, | 50 | | 212224_at | ALDH1A1 |
| member A1 | | | | |
| plexin D1 | 51 | | 212235_at | PLXND1 |
| low density lipoprotein receptor- | 52 | | 212850_s_at | LRP4 |
| related protein 4 | | | | |
| p53-associated parkin-like | 53 | | 213204_at | PARC |
| cytoplasmic protein | | | | |
| hypothetical protein PP1665 | 54 | | 213343_s_at | PP1665 |
| odd-skipped-related 2A protein | 55 | | 213568_at | OSR2 |
| angiotensin II receptor-like 1 | 56 | | 213592_at | AGTRL1 |
| spermidine/spermine N1- | 57 | | 213988_s_at | SAT |
| acetyltransferase | | | | |
| transferrin | 58 | | 214063_s_at | TF |
| complement component 4A | 59 | | 214428_x_at | C4A |
| serum amyloid A2 | 60 | | 214456_x_at | SAA2 |
| orosomucoid 2 | 61 | | 214465_at | ORM2 |
| homeo box D11 | 62 | | 214604_at | HOXD11 |
| eyes absent homolog 1 (Drosophila) | 63 | 153, 154, 155 | 214608_s_at | EYA1 |
| similar to Human Ig rearranged | 64 | | 214669_x_at | na |
| gamma chain mRNA, V-J-C region | | | | |
| H factor (complement)-like 1 | 65 | | 215388_s_at | HFL1 |
| complement component 3 | 66 | | 217767_at | C3 |
| complement component 1, q | 67 | | 218232_at | C1QA |
| subcomponent, alpha polypeptide | | | | |
| glucosaminyl (N-acetyl) transferase 3, | 68 | | 219508_at | GCNT3 |
| mucin type | | | | |
| fibrinogen, gamma polypeptide | 69 | 156 | 219612_s_at | FGG |
| a disintegrin-like and metalloprotease | 70 | | 222162_s_at | ADAMTS1 |
| with thrombospondin type 1 | | | | |
| C-reactive protein, pentraxin-related | 71 | | 37020_at | CRP |
| hemopexin | 72 | | 39763_at | HPX |
| fascin homolog 1, actin-bundling | 73 | | 201564_s_at | FSCN1 |
| protein (Strongylocentrotus | | | | |
| purpuratus) | | | | |
| tenascin C (hexabrachion) | 74 | | 201645_at | TNC |
| family with sequence similarity 13, | 75 | | 202973_x_at | FAM13A1 |
| member A1 | | | | |
| aldehyde dehydrogenase 1 family, | 76 | | 203180_at | ALDH1A3 |
| member A3 | | | | |
| phospholipase A2, group IIA | 77 | | 203649_s_at | PLA2G2A |
| (platelets, synovial fluid) | | | | |
| phosphodiesterase 4B, cAMP-specific | 78 | 157 | 203708_at | PDE4B |
| (phosphodiesterase E4 dunce | | | | |
| homolog, Drosophila) | | | | |
| biliverdin reductase A | 79 | | 203771_s_at | BLVRA |
| IGF-II mRNA-binding protein 3 | 80 | | 203819_s_at | IMP-3 |
| mitogen-activated protein kinase | 81 | | 203837_at | MAP3K5 |
| kinase kinase 5 | | | | |
| mucin 2, intestinal/tracheal /// mucin | 82 | | 204673_at | MUC2 |
| 2, intestinal/tracheal | | | | |
| solute carrier family 26 (sulfate | 83 | | 205097_at | SLC26A2 |
| transporter), member 2 | | | | |
| glutamate decarboxylase 1 (brain, | 84 | | 205278_at | GAD1 |
| 67 kDa) | | | | |
| pleiomorphic adenoma gene 1 | 85 | | 205372_at | PLAG1 |
| KIAA0672 gene product | 86 | | 205414_s_at | KIAA0672 |
| cpiregulin | 87 | | 205767_at | EREG |
| dead ringer-like 1 (Drosophila) | 88 | | 205865_at | DRIL1 |
| cathepsin E | 89 | 181 | 205927_s_at | CTSE |
| cell growth regulator with EF hand | 90 | | 205937_at | CGREF1 |
| domain 1 | | | | |
| sciellin | 91 | 158 | 206884_s_at | SCEL |
| PTK7 protein tyrosine kinase 7 | 92 | 159, 160, 161, | 207011_s_at | PTK7 |
| | | 162 | | |
| cyclin-dependent kinase inhibitor 2A | 93 | 163, 164, 165 | 207039_at | CDKN2A |
| (melanoma, p16, inhibits CDK4) | | | | |
| chromosome Y open reading frame 14 | 94 | | 207063_at | CYorf14 |
| leukocyte-associated Ig-like receptor 2 | 95 | 166 | 207509_s_at | LAIR2 |
| Mediterranean fever | 96 | | 208262_x_at | MEFV |
| keratin 6A | 97 | 167, 168 | 209125_at | KRT6A |
| spondin 1, (f-spondin) extracellular | 98 | 169 | 209436_at | SPON1 |
| matrix protein | | | | |
| homeo box A9 | 99 | 170 | 209905_at | HOXA9 |
| granzyme B (granzyme 2, cytotoxic | 100 | | 210164_at | GZMB |
| T-lymphocyte-associated serine | | | | |
| esterase 1) | | | | |
| interleukin 1 receptor, type II | 101 | 171, 172 | 211372_s_at | IL1R2 |
| immunoglobulin heavy constant | 102 | 173 | 211430_s_at | IGHG3 |
| gamma 3 (G3m marker) | | | | |
| protein O-fucosyltransferase 1 | 103 | 174 | 212349_at | POFUT1 |
| lipocalin 2 (oncogene 24p3) | 104 | | 212531_at | LCN2 |
| paraneoplastic antigen | 105 | | 213230_at | HUMPPA |
| SATB family member 2 | 106 | 175 | 213435_at | SATB2 |
| G protein-coupled receptor 49 | 107 | | 213880_at | GPR49 |
| spondin 1, (f-spondin) extracellular | 108 | 176 | 213994_s_at | SPON1 |
| matrix protein | | | | |
| immunoglobulin lambda joining 3 | 109 | 177 | 214677_x_at | IGLJ3 |
| — | 110 | | 216910_at | — |
| family with sequence similarity 13, | 111 | | 217047_s_at | FAM13A1 |
| member A1 | | | | |
| chromosome 14 open reading frame | 112 | 178, 179, 180 | 217988_at | C14orf18 |
| 18 | | | | |
| H2A histone family, member Y2 | 113 | | 218445_at | H2AFY2 |
| heparan sulfate (glucosamine) 3-O- | 114 | | 219697_at | HS3ST2 |
| sulfotransferase 2 | | | | |
| LBP protein; likely ortholog of mouse | 115 | | 219735_s_at | LBP-9 |
| CRTR-1 | | | | |
| frizzled homolog 10 (Drosophila) | 116 | | 219764_at | FZD10 |
| deafness locus associated putative | 117 | | 220482_s_at | DELGEF |
| guanine nucleotide exchange factor | | | | |
| aryl hydrocarbon receptor nuclear | 118 | | 220658_s_at | ARNTL2 |
| translocator-like 2 | | | | |
| Homo sapiens transcribed sequence | 119 | | 222347_at | — |
| with weak similarity to protein | | | | |
| ref: NP_009056.1 (H. sapiens) | | | | |
| ubiquitously transcribed | | | | |
| tetratricopeptide repeat gene, Y | | | | |
| chromosome; Ubiquitously | | | | |
| transcribed TPR gene on Y | | | | |
| chromosome [Homo sapiens] | | | | |
| CDW52 antigen (CAMPATH-1 | 120 | | 34210_at | CDW52 |
| antigen) | | | | |

Key:
\textcircled{1} Gene name
\textcircled{2} Variants represented in SEQ ID NO
3 Affy No.

The concept “variant,” in this invention, regarding the 120 genes disclosed here, comprises splice variants and also homologous or highly homologous genes.

1. A method for
(a) detecting a colorectal carcinoma;
(b) predicting metastases dependent on a primary colorectal carcinoma; or
(c) predicting the response of metastases to a 5-fluorouracil-containing chemotherapy;
comprising determining the gene expression profile of at least one marker gene selected from the group consisting of: SEQ ID NOs: 1-181 and combinations thereof.

2. The method according to claim 1, in which the expression profiles of at least two of the marker genes are determined and compared with a reference.

3. The method for detecting a colorectal carcinoma according to claim 1, in which the expression levels of at least two of the marker genes are determined and compared with the average expression levels of the genes from normal intestinal mucosa.

4. The method for detecting a colorectal carcinoma according to claim 1, in which the expression profiles of at least two marker genes selected from the group consisting of: SEQ ID NOs: 1-72 and SEQ ID NOs: 121-156, and combinations thereof, are determined.

5. The method for detecting a colorectal carcinoma according to claim 1, in which the expression profiles of at least two marker genes selected from the group consisting of: SEQ ID NOs: 10, 14, 25, 41, 52, 63, 68, 73-120, 136, 137, 138, 153, 154, 155 and 157-181, and combinations thereof, are determined.

6. The method for detecting a colorectal carcinoma according to claim 1, in which the expression profiles of at least two marker genes selected from the group consisting of: SEQ ID NOs: 2, 7, 8, 12, 19, 21, 25, 33, 38, 41, 45, 49, 50, 51, 54, 66, 68, 70, 78, 83, 85, 86, 92, 99, 101, 103, 104, 105, 109, 112, 114, 115, 118, 122, 124, 136-140, 157, 159-162, 170, 171, 172, 174 and 177-180, and combinations thereof, are determined.

7. The method for detecting a colorectal carcinoma according to claim 1, in which the expression profiles of at least two marker genes selected from the group consisting of: SEQ ID NOs: 2, 7, 8, 12, 19, 21, 25, 33, 38, 41, 45, 49, 50, 51, 54, 66, 68, 70, 122, 124 and 136-140, and combinations thereof, are determined.

8. The method for detecting a colorectal carcinoma according to claim 1, in which the expression profiles of at least two marker genes selected from the group consisting of: SEQ ID NOs: 25, 41, 68, 78, 83, 85, 86, 92, 99, 101, 103, 104, 105, 109, 112, 114, 115, 118, 136-138, 157, 159-162, 170-172, 174 and 177-180, and combinations thereof, are determined.

9. The method for detecting a colorectal carcinoma according to claim 1, in which the expression profile of at least one of the three marker genes selected from the group consisting of: SEQ ID NOs: 25, 41, 68 and 136-138, and combinations thereof, is determined.

10. The method for predicting metastases dependent on a primary colorectal carcinoma according to claim 1, in which the expression levels or expression profiles in a sample of a primary colorectal carcinoma of at least two genes selected from the group consisting of: SEQ ID NOs: 1-181 are determined and compared with the expression levels in liver metastases, liver tissue, or both.

11. The method for predicting metastases dependent on a primary colorectal carcinoma according to claim 1, in which the expression profiles of at least two marker genes selected from the group consisting of: SEQ ID NOs: 1-72 or 121-156, and combinations thereof, are determined.

12. The method for predicting metastases dependent on a primary carcinoma according to claim 1, in which the expression profiles of at least two marker genes selected from the group consisting of: SEQ ID NOs: 10, 14, 25, 41, 52, 63, 68, 73-120, 136, 137, 138, 153, 154, 155 and 157-181, and combinations thereof, are determined.

13. The method for predicting metastases dependent on a primary colorectal carcinoma according to claim 1, in which the expression profiles of at least two marker genes selected from the group consisting of: SEQ ID NOs: 17, 19, 20, 22, 26, 29, 30, 31, 33, 35, 37, 40, 48, 58, 59, 64, 66, 69, 71, 72, 74, 109, 125, 133, 134, 135, 151, 152 and 177, and combinations thereof, are determined.

14. The method for predicting metastases dependent on a primary colorectal carcinoma according to claim 1, in which the expression profiles of at least two marker genes selected from the group consisting of: SEQ ID NOs: 17, 19, 20, 22, 26, 29, 30, 31, 33, 35, 37, 40, 48, 58, 59, 64, 66, 69, 71, 72, 125, 133, 134, 135, 151, 152 and 156, and combinations thereof, are determined.

15. The method for predicting metastases dependent on a primary colorectal carcinoma according to claim 1, in which the expression profiles of at least one of the two marker genes selected from the group consisting of: SEQ ID NOs: 74, 109 and 177, and combinations thereof, are determined.

16. The method for predicting the response of metastases to a 5-fluorouracil-containing chemotherapy according to claim 1, in which the expression levels, expression profiles, or both, in a sample of a metastasis of at least two of the 120 genes selected from the group consisting of: SEQ ID NO: 1-181 and combinations thereof, is determined and compared with the expression levels, expression profiles, or both, of responders, non-responders, or both.

17. The method for predicting the response of metastases to a 5-fluorouracil-containing chemotherapy according to claim 1, in which the expression profiles of at least two marker genes selected from the group consisting of: SEQ ID NOs: 1-72 or 121-156, and combinations thereof, are determined.

18. The method for predicting the response of metastases to a 5-fluorouracil-containing chemotherapy according to claim 1, in which the expression profiles of at least two marker genes selected from the group consisting of: SEQ ID NOs. 10, 14, 25, 41, 52, 63, 73-120, 136, 137, 138, 153, 154, 155, and 157-181, and combinations thereof, are determined.

19. The method for predicting the response of metastases to a 5-fluorouracil-containing chemotherapy according to claim 1, in which the expression profiles of at least two marker genes selected from the group consisting of: SEQ ID NOs: 6, 7, 8, 10, 13, 14, 24, 25, 41, 45, 46, 48, 52, 54, 60, 61, 63, 65, 66, 68, 73, 78, 79, 80, 89, 97, 98, 101, 104, 107, 108, 116, 117, 120, 122, 136-147, 151-155, 157, 167, 168, 169, 171, 172, 176 and 181, and combinations thereof, are determined.

20. The method for predicting the response of metastases to a 5-fluorouracil-containing chemotherapy according to claim 1, in which the expression profiles of at least two marker genes selected from the group consisting of: SEQ ID NOs: 6, 7, 8, 13, 24, 45, 46, 48, 52, 54, 60, 61, 65, 66, 73, 78, 79, 80, 89, 97, 98, 101, 104, 107, 108, 116, 117, 120, 122, 139-147, 151, 152, 157, 167, 168, 169, 171, 172, 176 and 181, and combinations thereof, are determined.

21. The method for predicting the response of metastases to a 5-fluorouracil-containing chemotherapy according to claim 1, in which the expression profiles of at least one marker gene selected from the group consisting of: SEQ ID NOs: 10, 14, 25, 41, 52, 63, 68, 136, 137, 138, 153, 154, 155, and combinations thereof, are determined.

22. The method according to claim 1, in which the determination of the gene expression profile comprises the determination of at least one marker gene which is at least 60% identical with one of the marker genes selected from the group consisting of: SEQ ID NOs: 1-120.

23. The method according to claim 1, wherein the expression profile of the marker genes is obtained from a sample from the patient.

24. The method according to claim 23, wherein the sample is selected from the group consisting of a tissue biopsy, peritoneal fluid, blood, urine, serum and stool, and combinations thereof.

25. The method according to claim 23, wherein the expression profile of the marker genes is determined by the measurement of the quantity of marker gene mRNA.

26. The method according to claim 25, wherein the quantity of marker gene mRNA is determined by gene chip technology, RT-PCR, Northern hybridization, dot blotting, in situ hybridization, or combinations thereof.

27. The method according to claim 23, wherein the expression profile of the marker gene is determined by the measurement of the quantity of a polypeptide encoded by the marker gene.

28. The method according to claim 27, wherein the quantity of polypeptide encoded by the marker gene is determined by ELISA, RIA, immuno-blotting, FACS, immunohistochemical methods, or combinations thereof.

29. The method according to claim 1, which comprises further comprising the following steps:
(a) determination of the gene expression profile as defined in claim 1 in a patient sample; and
(b) determination, with the help of the gene expression profile determined in step (a), whether the patient
i. suffers from a colorectal carcinoma;
ii. presents metastases subsequent to a colorectal carcinoma;
iii. responds to 5-FU-containing chemotherapy of the metastases, or combinations thereof.

30. A kit for carrying out the method according to claim 1, which kit comprises specific nucleotide probes, primer pairs, antibodies, aptamers, and combinations thereof for the determination of at least two of marker genes which are selected from the group consisting of: SEQ ID NO: 1-181, and combinations thereof, or for the determination of at least two gene products coded for by the marker genes which are selected from the group consisting of: SEQ ID NO: 1-181, and combinations thereof.

31. The kit according to claim 30, which kit is a diagnostic kit.

32. A method to determine whether the patient
(a) suffers from a colorectal carcinoma;
(b) presents metastases subsequent to a colorectal carcinoma;
(c) responds to 5-FU-containing chemotherapy of the metastases, or combinations thereof, comprising:
detecting one of more of the marker genes selected from the group consisting of: SEQ ID NO: 1 to 181, and combinations thereof.

33. A method of screening for drugs comprising screening a transgenic non-human animal which overexpresses or underexpresses one or more of the marker genes of claim 1.

34. A method of screening for drugs comprising screening a transgenic cell which overexpresses or underexpresses one or more of the marker genes of claim 1.

35. The method of claim 33 or 34, wherein a drug identified by the screen is used to treat colorectal carcinoma, metastases, or both.

36. The method according to claim 1, where the metastases are liver metastases.
